(19) United States
(12) Patent Application Publication
(10) Pub. No.: US 2025/0188519 A1
(43) Pub. Date: Jun. 12, 2025

(54) ANALYZING VARIANT SEQUENCES USING IN SITU OR SPATIAL ASSAYS

(71) Applicant: 10x Genomics, Inc., Pleasanton, CA (US)

(72) Inventors: Marco MIGNARDI, Sundbyberg (SE); Jessica ÖSTLIN, Stocksund (SE); Ruijie ZHANG, Milpitas, CA (US)

(73) Assignee: 10x Genomics, Inc., Pleasanton, CA (US)

(21) Appl. No.: 18/960,940

(22) Filed: Nov. 26, 2024

Related U.S. Application Data
(60) Provisional application No. 63/606,950, filed on Dec. 6, 2023.

(51) Int. Cl. C12Q 1/6827; C12N 15/10; C12Q 1/6855; C12Q 1/6876

(52) U.S. Cl. CPC C12Q 1/6827; C12N 15/1096; C12Q 1/6855; C12Q 1/6876; C12Q 2600/156

(57) ABSTRACT

The present disclosure relates in some aspects to methods for analyzing target nucleic acids and their spatial locations in a biological sample. In some aspects, the presence/absence, amount, and/or identity of variant sequences (e.g., single nucleotide variations such as SNPs or point mutations) in a plurality of target nucleic acids in a cell or tissue sample are analyzed in situ in the sample or using a spatial array. Also provided are oligonucleotides, sets of oligonucleotides, compositions, and kits for use in accordance with the methods.

### CROSS-REFERENCE TO RELATED APPLICATIONS

This application claims benefit of and priority to U.S. Provisional Application No. 63/606,950, filed on Dec. 6, 2023, entitled “Analyzing Variant Sequences Using In Situ or Spatial Assays,” which is herein incorporated by reference in its entirety for all purposes.

### FIELD

The present disclosure relates in some aspects to methods and compositions for in situ and/or spatial array-based analysis of nucleic acids and variant sequences therein in biological samples, such as multiplex genotyping of single nucleotide differences in target nucleic acid molecules of a cell or tissue sample using in situ detection or spatial array-based analysis.

### BACKGROUND

Methods are available for detecting nucleic acids present in a biological sample. For instance, advances in single molecule fluorescent in situ hybridization (smFISH) have enabled nanoscale-resolution imaging of RNA in cells and tissues. However, analysis of short sequences (e.g., single nucleotide differences such as single nucleotide polymorphisms (SNPs) or point mutations) on individual transcripts in samples (e.g., a tissue section) has remained challenging. Improved methods for identifying short variant sequences and analyzing their spatial profiles (e.g., sequence identity, spatial location, and/or abundance) in cell or tissue samples are needed. Provided herein are methods, compositions, and kits that address such and other needs.

### BRIEF SUMMARY

Certain methods for detecting variant sequences aim to discriminate a variant sequence (e.g., a particular SNP or point mutation) at the probe hybridization/ligation step. For instance, padlock probes with only one base difference in the arms (e.g., at the 3′ or 5′ end of the probes) targeting a single nucleotide difference in target nucleic acids can compete with each other for hybridization to a target sequence. The best matching probe can outcompete the other probes, become more stably hybridized to a target molecule, and can then be circularized using a ligase and the target sequence as a ligation template. The ligated probe can be amplified using rolling circle amplification (RCA) (e.g., as illustrated in Scenario 1 and Scenario 2 in FIG. 1) and detected by various readout means. However, assays using probe hybridization/ligation to discriminate variant sequences can suffer from low specificity for multiple reasons, including properties of the ligase and/or the target nucleic acid. Low ligase fidelity can result in formation and detection of a ligation product (and subsequent amplicons of the ligation product), even when the sequence of interest does not match an interrogatory region of a probe, producing a high level of background or false positive results. For example, RNA templated ligases can tolerate some mismatches. In addition, ligases can have a strong base preference and probe end bias. Moreover, in the case of padlock probes targeting a single nucleotide difference, the single nucleotide is usually targeted by one arm, while the other arm covers a common region (e.g., a conserved region) among nucleic acid molecules containing different bases at the single nucleotide position of interest. As a result, on the one hand, one arm of a first padlock probe could hybridize to the common region in a particular target nucleic acid molecule, but the other arm of the first padlock probe does not fully match the single nucleotide position in the particular target nucleic acid molecule. Due to low ligase fidelity on RNA templates, the nonspecifically hybridized probes can nevertheless be ligated and amplified by RCA (e.g., as illustrated in Scenario 3 and Scenario 4 in FIG. 1). On the other hand, one arm of a second padlock probe could perfectly match the single nucleotide position in the target nucleic acid molecule, but the other arm of the second padlock probe cannot hybridize to the common region which is occupied by the first padlock probe or another probe. This is either ligated to a two-probe chimera that cannot be amplified by RCA, or probe hybridization can become unstable so that none of the padlock probes generates a ligation product and subsequently a detectable RCP, which can lead to a drop in detection efficiency and/or accuracy. Improved methods for analyzing nucleic acids present in a biological sample, such as for detecting a single nucleotide of interest using in situ or spatial array-based detection, are needed.

In some aspects, herein is provided a method for analyzing a biological sample or detecting a sequence in a biological sample. In some aspects, the method comprises: a) contacting the biological sample with a first probe or probe set, wherein the first probe or probe set comprises a first probe region and a second probe region that bind to a first target sequence and a second target sequence, respectively, in a target RNA in the biological sample, wherein the first and second target sequences flank a gap sequence in the target RNA, and wherein the gap sequence comprises a variant sequence; b) performing a gap-fill reaction on the first probe or probe set to generate a gap-filled probe or probe set and circularizing the gap-filled probe or probe set; c) using a polymerase to amplify the circularized gap-filled probe or probe set to generate a rolling circle amplification product (RCP) in the biological sample; d) contacting the biological sample with a second probe or probe set that binds to the RCP, wherein the second probe or probe set comprises an interrogatory region for interrogating the variant sequence in the RCP; e) ligating the second probe or probe set to generate a ligation product comprising the interrogatory region which comprises a sequence complementary to the variant sequence in the RCP; and f) detecting and/or analyzing the ligation product to determine a location of the target RNA comprising the variant sequence in the biological sample.

In some embodiments, performing the gap-fill reaction comprises contacting the biological sample with a library of splint oligonucleotides, wherein each splint oligonucleotide comprises i) ligatable ends; and ii) a hybridization region complementary to one of a plurality of different sequences, wherein a splint oligonucleotide of the library of splint oligonucleotides that is complementary to the gap sequence is ligated to the first probe or probe set. In some embodiments, the splint oligonucleotide comprises a 3′ hydroxyl group and a 5′ phosphate group. Optionally, in some embodiments, the splint nucleotide comprises one or more ribonucleotide residues at and/or near its 3′ end and/or a 5′ flap configured to be cleaved by a structure-specific endonuclease. In some embodiments, the splint oligonucleotide and/or the gap sequence is between about 2 and about 40 nucleotides in length. In some embodiments, the variant sequence is at the 3′ or 5′ end of the gap sequence, and/or the sequence complementary to the variant sequence is at the 5′ or 3′ end of the splint oligonucleotide. In some embodiments, the variant sequence is at least 1, 2, 3, 4, 5, 6, 7, 8, 9, or more phosphodiester bonds from the 3′ or 5′ end of the gap sequence, and/or the sequence complementary to the variant sequence is at least 1, 2, 3, 4, 5, 6, 7, 8, 9, or more phosphodiester bonds from the 5′ or 3′ end of the splint oligonucleotide. Optionally, in some embodiments, the variant sequence is at or near the central nucleotide(s) of the gap sequence and/or the sequence complementary to the variant sequence is at or near the central nucleotide(s) of the splint oligonucleotide.

In some embodiments, the variant sequence comprises a nucleotide variation, a nucleotide polymorphism, a mutation, a substitution, an insertion, a deletion, a translocation, a duplication, an inversion, and/or a repetitive sequence. In some embodiments, the variant sequence comprises two or more nucleotide residues. In some embodiments, the variant sequence is a single nucleotide. In some embodiments, the variant sequence comprises a single nucleotide variation (SNV), a single nucleotide polymorphism (SNP), a point mutation, a single nucleotide substitution, a single nucleotide insertion, or a single nucleotide deletion.

In some embodiments, the splint oligonucleotide is ligated to the first probe or probe set using the target RNA as a template and a ligase having an RNA-templated DNA or RNA ligase activity. In some embodiments, the ligase is a Chlorella virus DNA ligase (PBCV DNA ligase) or a T4 RNA ligase. In some embodiments, the ligase is a PBCV-1 DNA ligase or a T4 RNA ligase 2.

In some embodiments, the library of splint oligonucleotides comprises at least or about 2, at least or about 5, at least or about 10, at least or about 15, at least or about 20, at least or about 25, at least or about 30, at least or about 35, at least or about 40, at least or about 45, at least or about 50, or more splint oligonucleotides of different hybridization region sequences. In some embodiments, the molar concentration of the library of splint oligonucleotides is about equal to or about 2, about 4, about 8, about 10, or more times the molar concentration of the first probe or probe set. In some embodiments, the method comprises washing the biological sample after contacting with the library of splint oligonucleotides. In some embodiments, the washing is performed under less than stringent conditions.

In some embodiments, performing the gap-fill reaction comprises using a gap-fill polymerase to extend an end of the first probe or probe set using the target RNA as a template to generate an extended probe, wherein the extended probe is ligated to another end of the first probe or probe set. In some embodiments, the gap-fill polymerase has no or little strand displacement activity. In some embodiments, the gap-fill polymerase incorporates one or more deoxyribonucleotide residues and/or one or more ribonucleotide residues into a 3′ end of the first probe or probe set to generate the extended probe. In some instances, the extended probe comprises one or more ribonucleotide residues at and/or near its 3′ end. In some embodiments, the extended probe is ligated to the first probe or probe set using the target RNA as a template and a ligase having an RNA-templated DNA or RNA ligase activity. In some embodiments, the ligase is a Chlorella virus DNA ligase (PBCV DNA ligase) or a T4 RNA ligase. Optionally, in some embodiments, the ligase is a PBCV-1 DNA ligase or a T4 RNA ligase 2. In some embodiments, the polymerase to amplify the circularized gap-filled probe or probe set is a Phi29 polymerase or a Bst polymerase.

In some embodiments, the second probe or probe set is DNA. In some embodiments, the variant sequence comprises a single nucleotide of interest, the interrogatory region in the second probe or probe set comprises a nucleic acid residue complementary to the single nucleotide of interest, and the nucleic acid residue is no more than 1, 2, 3, 4, or 5 phosphodiester bonds from a 3′ or 5′ end of the second probe or probe set. In some embodiments, the interrogatory region comprises a 3′ terminal nucleic acid residue that is complementary to the single nucleotide of interest. In some embodiments, the interrogatory region comprises a 5′ terminal nucleic acid residue that is complementary to the single nucleotide of interest. In some embodiments, the second probe or probe set comprises a 5′ flap configured to be cleaved by a structure-specific endonuclease to release the 5′ flap from the second probe or probe set and allow ligation of the second probe or probe set. In some embodiments, the second probe or probe set comprises a 3′ terminal nucleic acid residue complementary to the single nucleotide of interest, and the released 5′ flap comprises a 3′ terminal nucleic acid residue complementary to the single nucleotide of interest. In some embodiments, the second probe or probe set comprises a 3′ terminal nucleic acid residue complementary to the nucleotide immediately 3′ to the single nucleotide of interest, and after cleaving the 5′ flap, the second probe or probe set comprises a 5′ terminal nucleic acid residue complementary to the single nucleotide of interest.

In some embodiments, the second probe or probe set is ligated using a ligase having a DNA-templated DNA ligase activity. In some embodiments, the second probe or probe set is ligated using a ligase selected from the group consisting of a Thermus thermophilus (Tth) DNA ligase, a Thermus aquaticus (Taq) DNA ligase, a T3 DNA ligase, a T4 DNA ligase, a T7 DNA ligase, an E. coli DNA ligase, a 9°N DNA ligase, a Chlorella virus DNA ligase (PBCV DNA ligase), and a T4 RNA ligase 2.

In some embodiments, the first probe or probe set does not comprise a barcode region comprising one or more barcode sequences associated with the target RNA or a sequence thereof. In some embodiments, the first probe or probe set comprises a barcode region comprising one or more barcode sequences associated with the target RNA or a sequence thereof, wherein the barcode region is not complementary to the target RNA or sequence thereof.

In some embodiments, the first and second probe regions are common among a plurality of first probes or probe sets each targeting a molecule comprising a different variant sequence of the target RNA. In some embodiments, the first probe region is 3′ or 5′ to the second probe region in the first probe or probe set. In some embodiments, the first probe region and the second probe region in the first probe or probe set are equal in length, or the first probe region is shorter or longer than the second probe region. In some embodiments, the first probe region and/or the second probe region in the first probe or probe set is between about 5 and about 50 nucleotides in length. In some embodiments, the first probe region and/or the second probe region in the first probe or probe set is between about 15 and about 25 nucleotides in length. Optionally, in some embodiments, the first probe region and/or the second probe region is about 20 nucleotides in length. In some embodiments, the first probe region and/or the second probe region in the first probe or probe set comprises one or mor ribonucleotide residues at and/or near its 3′end and/or a 5′ flap configured to be cleaved by a structure-specific endonuclease.

In some embodiments, the second probe or probe set comprises a barcode region comprising one or more barcode sequences associated with the target RNA or a sequence thereof, wherein the barcode region is not complementary to the target RNA or sequence thereof.

In some embodiments, the detecting of the ligation product (or a derivative thereof) comprises contacting the biological sample with detectably labeled probes in sequential cycles, wherein in each cycle, a detectably labeled probe is directly or indirectly bound to a barcode sequence in a barcode region of the ligation product, and a signal or absence thereof associated with the detectably labeled probe is recorded at a location in the biological sample, thereby generating a signal code sequence corresponding to the barcode region. In some instances, the signal code sequence comprises the signal or absence thereof recorded at the location in each of the sequential cycles and wherein the signal code sequence identifies the variant sequence of the target RNA at the location in the biological sample. In some embodiments, the barcode is associated with the variant sequence in the target RNA. In some instances, in each cycle, the detectably labeled probe is hybridized to an intermediate probe which in turn hybridizes to the barcode region. In some embodiments, in two or more of the sequential cycles, the intermediate probes are hybridized to the same barcode sequence or different barcode sequences in the barcode region. In some embodiments, in two or more of the sequential cycles, the intermediate probes are hybridized to the same barcode sequence in the barcode region. In some embodiments, in two or more of the sequential cycles, the intermediate probes are hybridized to different barcode sequences in the barcode region. In some embodiments, in each of the sequential cycles, the intermediate probe is hybridized to the same barcode sequence or a different barcode sequence in the barcode region. In some instances, the detectably labeled probe hybridizes directly to the barcode region. In some instances, in each cycle, the detectably labeled probe is hybridized to the barcode region. In some embodiments, in two or more of the sequential cycles, the detectably labeled probes are hybridized to different barcode sequences in the barcode region. In some embodiments in each of the sequential cycles, the detectably labeled probe is hybridized to a different barcode sequence in the barcode region.

In some embodiments, the second probe or probe set comprises the interrogatory region and a constant region complementary to the target RNA. In some embodiments, the constant region is common among a plurality of second probes or probe sets each comprising a different interrogatory region for a different variant sequence of the target RNA. In some embodiments, the interrogatory region is 3′ or 5′ to the constant region in the second probe or probe set. In some embodiments, the interrogatory region and the constant region in the second probe or probe set are equal in length, or the interrogatory region is shorter or longer than the constant region. In some embodiments, the interrogatory region and/or the constant region in the second probe or probe set is between about 5 and about 50 nucleotides in length. In some embodiments, the interrogatory region and/or the constant region in the second probe or probe set is between about 15 and about 25 nucleotides in length. Optionally, in some embodiments the interrogatory region and/or the constant region is about 20 nucleotides in length. In some embodiments, the interrogatory region and/or the constant region in the second probe or probe set comprises a 5′ flap configured to be cleaved by a structure-specific endonuclease.

In some aspects, provided herein is a method comprising: a) contacting the biological sample with a first probe or probe set, wherein the first probe or probe set comprises a first probe region and a second probe region that bind to a first target sequence and a second target sequence, respectively, in a target RNA in the biological sample, wherein the first and second target sequences flank a gap sequence in the target RNA, and wherein the gap sequence comprises a variant sequence; b) performing a gap-fill reaction on the first probe or probe set bound to the target RNA to generate a gap-filled probe or probe set and circularizing the gap-filled probe or probe set, c) using a polymerase to amplify the circularized gap-filled probe or probe set to generate a rolling circle amplification product (RCP) in the biological sample; d) contacting the biological sample with a second probe or probe set that binds to the RCP, wherein the second probe or probe set comprises i) an interrogatory region for interrogating the variant sequence in the RCP, and ii) a barcode region; e) ligating the second probe or probe set bound to the RCP to generate a ligation product when the interrogatory region comprises a sequence complementary to the variant sequence in the RCP; and f) contacting the biological sample with detectably labeled probes in sequential cycles, wherein in each cycle, a detectably labeled probe is directly or indirectly bound to a barcode sequence in the barcode region and a signal or absence thereof associated with the detectably labeled probe is recorded at a location in the biological sample, thereby generating a signal code sequence corresponding to the barcode region, wherein the signal code sequence comprises the signal or absence thereof recorded at the location in each of the sequential cycles. In some aspects, the signal code sequence identifies the variant sequence of the target RNA at the location in the biological sample. In some aspects, the signal code sequence is used to identify the variant sequence of the target RNA at the location in the biological sample.

In some aspects, provided herein is a method for analyzing a biological sample, comprising: a) contacting the biological sample with a first probe or probe set, wherein the first probe or probe set comprises a first probe region and a second probe region that bind to a first target sequence and a second target sequence, respectively, in a target RNA in the biological sample, wherein the first and second target sequences flank a gap sequence in the target RNA, and wherein the gap sequence comprises a variant sequence; b) performing a gap-fill reaction on the first probe or probe set to generate a gap-filled probe or probe set and circularizing the gap-filled probe or probe set, c) using a polymerase to amplify the circularized gap-filled probe or probe set to generate a rolling circle amplification product (RCP) in the biological sample; d) contacting the biological sample with a second probe or probe set that binds to the RCP, wherein the second probe or probe set comprises i) an interrogatory region for interrogating the variant sequence in the RCP, and ii) a barcode region; e) ligating the second probe or probe set to generate a ligation product when the interrogatory region comprises a sequence complementary to the variant sequence in the RCP; removing molecules of probes or probe sets that are not ligated due to a mismatch with the variant sequence in the RCP; contacting the biological sample with detectably labeled probes in sequential cycles, wherein in each cycle, a detectably labeled probe is directly or indirectly bound to a barcode sequence in the barcode region and a signal or absence thereof associated with the detectably labeled probe is recorded at a location in the biological sample, thereby generating a signal code sequence corresponding to the barcode region, wherein the signal code sequence comprises the signal or absence thereof recorded at the location in each of the sequential cycles; and h) using the signal code sequence to identify the variant sequence of the target RNA at the location in the biological sample.

In some embodiments, the barcode region in the second probe or probe set is associated with the variant sequence in the target RNA. In some embodiments, the biological sample is contacted with a plurality of second probes or probe sets each comprising a different interrogatory region for a different variant sequence of the target RNA and a different barcode region corresponding to the different variant sequence. In some instances, the biological sample is washed to remove molecules of a second probe or probe set that is not ligated due to a mismatch with the variant sequence in the RCP. In some embodiments, the first probe or probe set does not comprise a barcode region comprising one or more barcode sequences associated with the target RNA or a sequence thereof. In some embodiments, the first probe or probe set comprises a barcode region comprising one or more barcode sequences associated with the target RNA or a sequence thereof but not associated with the variant sequence.

In some embodiments, the detectably labeled probe is hybridized to an intermediate probe which in turn hybridizes to the barcode region. In some embodiments, the intermediate probes are hybridized to the same barcode sequence or different barcode sequences in the barcode region. In some embodiments, in two or more of the sequential cycles, the intermediate probes are hybridized to the same barcode sequence or different barcode sequences in the barcode region. In some embodiments, in each of the sequential cycles, the intermediate probe is hybridized to the same barcode sequence or a different barcode sequence in the barcode region. In some embodiments, in each cycle, the detectably labeled probe is hybridized to the barcode region. In some embodiments, in two or more of the sequential cycles, the detectably labeled probes are hybridized to different barcode sequences in the barcode region. In some embodiments, in each of the sequential cycles, the detectably labeled probe is hybridized to a different barcode sequence in the barcode region.

In some embodiments, the barcode region comprises two or more non-overlapping barcode sequences. In some embodiments, the barcode region comprises two or more overlapping barcode sequences. In some embodiments, each pair of adjacent barcode sequences in the barcode region are partially overlapping. In some embodiments, the ligation product of the second probe or probe set is circular. In some embodiments, the ligation product of the second probe or probe set is not circular.

In some embodiments, provided herein is a method comprising: a) contacting the biological sample with a first probe or probe set, wherein the first probe or probe set comprises i) a first probe region and a second probe region that bind to a first target sequence and a second target sequence, respectively, in a target RNA in the biological sample, and ii) a complement of a first barcode region, wherein the first and second target sequences flank a gap sequence in the target RNA, and wherein the gap sequence comprises a variant sequence; b) performing a gap-fill reaction on the first probe or probe set bound to the target RNA to generate a gap-filled probe or probe set and circularizing the gap-filled probe or probe set; c) using a polymerase to amplify the circularized gap-filled probe or probe set to generate a rolling circle amplification product (RCP) in the biological sample; d) contacting the biological sample with a second probe or probe set that binds to the RCP, wherein the second probe or probe set comprises i) an interrogatory region for interrogating the variant sequence in the RCP, and ii) a second barcode region; e) ligating the second probe or probe set bound to the RCP to generate a ligation product when the interrogatory region comprises a sequence complementary to the variant sequence in the RCP; and f) contacting the biological sample with detectably labeled probes in sequential cycles, wherein in each cycle, a detectably labeled probe is directly or indirectly bound to a barcode sequence in the first or second barcode region and a signal or absence thereof associated with the detectably labeled probe is recorded at a location in the biological sample, thereby generating a signal code sequence corresponding to the first and second barcode regions, wherein the signal code sequence comprises the signal or absence thereof recorded at the location in each of the sequential cycles. In some embodiments, the signal code sequence is associated with or identifies the variant sequence of the target RNA at the location in the biological sample. In some embodiments, the signal code sequence is used to identify the variant sequence of the target RNA at the location in the biological sample. In some instances, the barcode region consists of one barcode sequence corresponding to a base selected from the group consisting of A, T, C, and G.

In some embodiments, provided herein is a method for analyzing a biological sample, comprising: a) contacting the biological sample with a first probe or probe set, wherein the first probe or probe set comprises i) a first probe region and a second probe region that bind to a first target sequence and a second target sequence, respectively, in a target RNA in the biological sample, and ii) a complement of a first barcode region, wherein the first and second target sequences flank a gap sequence in the target RNA, and wherein the gap sequence comprises a variant sequence; b) performing a gap-fill reaction on the first probe or probe set to generate a gap-filled probe or probe set and circularizing the gap-filled probe or probe set; c) using a polymerase to amplify the circularized gap-filled probe or probe set to generate a rolling circle amplification product (RCP) in the biological sample; d) contacting the biological sample with a second probe or probe set that binds to the RCP, wherein the second probe or probe set comprises i) an interrogatory region for interrogating the variant sequence in the RCP, and ii) a second barcode region; e) ligating the second probe or probe set to generate a ligation product when the interrogatory region comprises a sequence complementary to the variant sequence in the RCP; f) removing molecules of probes or probe sets that are not ligated due to a mismatch with the variant sequence in the RCP; g) contacting the biological sample with detectably labeled probes in sequential cycles, wherein in each cycle, a detectably labeled probe is directly or indirectly bound to a barcode sequence in the first or second barcode region and a signal or absence thereof associated with the detectably labeled probe is recorded at a location in the biological sample, thereby generating a signal code sequence corresponding to the first and second barcode regions, wherein the signal code sequence comprises the signal or absence thereof recorded at the location in each of the sequential cycles; and h) using the signal code sequence to identify the variant sequence of the target RNA at the location in the biological sample.

In some embodiments, the first barcode region comprises one or more barcode sequences associated with the target RNA or a sequence thereof but not associated with the variant sequence. In some embodiments, the first barcode region comprises two or more non-overlapping barcode sequences. In some embodiments, the first barcode region comprises two or more overlapping barcode sequences. In some embodiments, each pair of adjacent barcode sequences in the first barcode region are partially overlapping.

In some embodiments, the second barcode region is associated with the variant sequence in the target RNA. In some embodiments, the biological sample is contacted with a plurality of second probes or probe sets each comprising a different interrogatory region for a different variant sequence of the target RNA and a different second barcode region corresponding to the different variant sequence. In some instances, the method comprises removing molecules of probes or probe sets that are not ligated due to a mismatch with the variant sequence in the RCP. In some embodiments, the second barcode region consists of one barcode sequence corresponding to a base selected from the group consisting of A, T, C, and G. In some embodiments, the barcode region corresponding to the base is common among a plurality of second probes or probe sets targeting different target RNAs or different sequences of the same target RNA.

In some embodiments, in each cycle, the detectably labeled probe is hybridized to an intermediate probe which in turn hybridizes to the first barcode region or the second barcode region. In some embodiments, in two or more sequential cycles, the intermediate probes are hybridized to the same barcode sequence or different barcode sequences in the first barcode region or the second barcode region. In some embodiments, in each of the sequential cycles for decoding the first barcode region, the intermediate probe is hybridized to the same barcode sequence or a different barcode sequence in the first barcode region. In some embodiments, in each cycle, the detectably labeled probe is hybridized to the first barcode region or the second barcode region. In some embodiments, in two or more of the sequential cycles, the detectably labeled probes are hybridized to different barcode sequences in the first barcode region or different barcode regions in the second barcode region. In some embodiments, in each of the sequential cycles for decoding the first barcode region, the detectably labeled probe is hybridized to a different barcode sequence in the first barcode region. In some embodiments, each pair of adjacent barcode sequences in the first barcode region are partially overlapping. In some embodiments, the ligation product of the second probe or probe set is circular. In some embodiments, the ligation product of the second probe or probe set is not circular.

In some embodiments, herein is provided a method for analyzing a biological sample, comprising a) contacting the biological sample with a first probe or probe set, wherein the first probe or probe set comprises a first probe region and a second probe region that bind to a first target sequence and a second target sequence, respectively, in a target RNA in the biological sample, wherein the first and second target sequences flank a gap sequence in the target RNA, and wherein the gap sequence comprises a variant sequence; b) performing a gap-fill reaction on the first probe or probe set to generate a gap-filled probe or probe set and circularizing the gap-filled probe or probe set; c)_using a polymerase to amplify the circularized gap-filled probe or probe set to generate a rolling circle amplification product (RCP) in the biological sample; d) contacting the biological sample with a second probe set that binds to the RCP, wherein the second probe set comprises an interrogatory region for interrogating the variant sequence in the RCP; e) ligating the second probe set to generate a ligation product when the interrogatory region comprises a sequence complementary to the variant sequence in the RCP; f) providing a plurality of capture probes to directly or indirectly capture the ligation product, wherein the plurality of capture probes are joined directly or indirectly to a substrate, and wherein a capture probe of the plurality of capture probes comprises: i) a capture domain capable of capturing the ligation product, and ii) a spatial barcode; g) generating a spatially labeled polynucleotide comprising (i) a sequence of the ligation product or complement thereof and (ii) a sequence of the spatial barcode or complement thereof; and h) determining a sequence of the spatially labeled polynucleotide or a complement thereof to identify the variant sequence of the target RNA at one or more locations in the biological sample. In some aspects, the first probe or probe set bound to the target RNA are gap-filled and circularized. In some aspects, the second probe set bound to the RCP are ligated.

In some embodiments, the second probe set comprises a first molecule comprising a functional sequence and a second molecule comprising a capture sequence that is configured to be hybridized or ligated to the capture domain of the capture probe. In some embodiments, the functional sequence in the first molecule comprises a primer binding sequence or a complement thereof. In some embodiments, the functional sequence in the first molecule is in a 5′ overhang upon hybridization of the first molecule to the RCP. In some embodiments, the capture sequence in the second molecule is in a 3′ overhang upon hybridization of the second molecule to the RCP. In some embodiments, the capture sequence in the second molecule comprises a 3′ polyadenine sequence. In some embodiments, the capture domain in the capture probe comprises a 3′ poly(dT) sequence.

In some embodiments, the substrate is a first substrate, the biological sample is on a second substrate, and the biological sample is sandwiched between the first and second substrates to allow capture of the ligation product by the capture domain. In some instances, each of the first and second substrates is a planar substrate. In some instances, the planar substrate is a slide or slip.

In some embodiments, the biological sample is processed to release the ligation product for capture by the capture domain. In some instances, the processing comprises permeabilizing and/or lysing the biological sample. In some embodiments, the method comprises cleaving the RCP at a single-stranded region not forming a duplex with the ligation product. In some embodiments, the method comprises denaturing the duplex between the RCP and the ligation product. In some embodiments, the spatially labeled polynucleotide or a portion thereof is removed from the substrate for determining its sequence. In some embodiments, the sequence of the spatially labeled polynucleotide or complement thereof is determined using nucleic acid sequencing. In some embodiments, the capture probe comprises a unique molecular identifier (UMI) region, a primer binding region, and/or an adapter region.

In some embodiments, herein is provided a method for analyzing a biological sample. In some embodiments, provided herein is a method for detecting a variant sequence in a target nucleic acid. In some instances, the method comprises: a) contacting the biological sample with a first probe or probe set, wherein the first probe or probe set comprises a first probe region and a second probe region that bind to a first target sequence and a second target sequence, respectively, in a target nucleic acid in the biological sample, wherein the first and second target sequences flank a gap sequence in the target nucleic acid, and wherein the gap sequence comprises a variant sequence; b) performing a gap-fill reaction on the first probe or probe set to generate a gap-filled probe or probe set and circularizing the gap-filled probe or probe set; c) using a polymerase to amplify the circularized gap-filled probe or probe set to generate a rolling circle amplification product (RCP) in the biological sample; d) contacting the biological sample with a second probe or probe set that binds to the RCP, wherein the second probe or probe set comprises an interrogatory region for interrogating the variant sequence in the RCP; e) ligating the second probe or probe set to generate a ligation product when the interrogatory region comprises a sequence complementary to the variant sequence in the RCP. In some cases, the method comprises detecting the ligation product to determine a location of the target RNA comprising the variant sequence in the biological sample. In some cases, the method comprises analyzing the ligation product to determine a location of the target RNA comprising the variant sequence in the biological sample.

In some embodiments, the 3′ end of the first probe is extended by a polymerase using the target nucleic acid as a template to generate an extended probe, and the 3′ end of the extended probe is ligated to the 5′ end of the first probe. In some embodiments, the biological sample is contacted with a library of splint oligonucleotides, wherein each splint oligonucleotide comprises: i) ligatable ends; and ii) a hybridization region complementary to one of a plurality of different sequences, wherein a splint oligonucleotide of the library of splint oligonucleotides that is complementary to the gap sequence is ligated to the first probe, thereby circularizing the first probe.

In some embodiments, the first probe or probe set comprises a first barcode region, and the method comprises detecting a barcode sequence in the first barcode region or a complement thereof at a location in the biological sample. In some embodiments, the second probe or probe set comprises a second barcode region, and the method comprises detecting a barcode sequence in the second barcode region or a complement thereof at a location in the biological sample.

In some instances, the biological sample is a cell or tissue sample. In some embodiments, the biological sample is a cell or tissue sample comprising cells or cellular components. In some embodiments, the biological sample is a tissue section. In some embodiments, the biological sample is a formalin-fixed, paraffin-embedded (FFPE) sample or a fresh frozen tissue sample. In some embodiments, the biological sample is fixed and/or permeabilized. In some embodiments, the biological sample is crosslinked and/or embedded in a matrix. In some instances, the matrix comprises a hydrogel. In some embodiments, the biological sample is cleared.

In some embodiments, the variant sequence is among a plurality of different sequences. In some embodiments, the variant sequence is a mutant sequence or a minor variant among a plurality of different variant sequences. In some embodiments, the variant sequence is a wildtype sequence or major variant among a plurality of different variant sequences. In some embodiments, the gap sequence comprises a genetic hotspot. In some embodiments, the gap sequence comprises two or more hotspot mutations.

In some embodiments, herein is provided a system or kit for analyzing the presence of a variant sequence in a target RNA at a location in a biological sample, comprising a) a plurality of first probes or probe sets, wherein: each first probe or probe set comprises a first probe region and a second probe region that is configured to bind to a first target sequence and a second target sequence, respectively, in a target RNA in the biological sample; the first and second target sequences flank a gap sequence in the target RNA; the gap sequence comprises a variant sequence; and the first probe or probe set is configured to be gap-filled, circularized, and amplified via rolling circle amplification to produce rolling circle amplification products (RCPs) containing multiple copies of a variant sequence; and b) a plurality of second probes or probe sets, wherein: each second probe or probe set is configured to hybridize to an RCP of the RCPs and comprises an interrogatory region comprising a sequence complementary to the variant sequence. In some embodiments, the kit further comprises c) one or more reagents for ligating the first probes or probe sets and/or the second probes or probe sets, and d) one or more reagents for rolling circle amplification. In some instances, the polymerase for RCA has little to no strand displacement activity.

In some embodiments, herein is provided a system or kit for analyzing the presence of a variant sequence in a target RNA at a location in a biological sample, comprising: a) a plurality of first probes or probe sets, wherein: each first probe or probe set comprises a first probe region and a second probe region that is configured to bind to a first target sequence and a second target sequence, respectively, in a target RNA in the biological sample; each first probe or probe set additionally optionally comprises a barcode sequence; the first and second target sequences flank a gap sequence in the target RNA; the gap sequence comprises a variant sequence; and the first probe or probe set is configured to be gap-filled, circularized, and amplified via rolling circle amplification to produce rolling circle amplification products (RCPs) containing multiple copies of a variant sequence and a barcode sequence; and b) a plurality of second probes or probe sets, wherein: each second probe or probe set is configured to hybridize to an RCP of the RCPs and comprises an interrogatory region comprising a sequence complementary to the variant sequence, and a barcode region; and c) a plurality of detectable probes, wherein: each detectable probe comprises a barcode sequence complementary to a barcode region from a second probe or probe set. In some embodiments, the system or kit further comprises e) one or more reagents for ligating the first probes or probe sets and/or the second probes or probe sets, and f) one or more reagents for rolling circle amplification. In some instances, the polymerase for RCA has little to no strand displacement activity.

In some embodiments, herein is provided a system or kit for analyzing the presence of a variant sequence in a target RNA at a location in a biological sample, comprising: a) a plurality of first probes or probe sets, wherein: each first probe or probe set comprises a first probe region and a second probe region that bind to a first target sequence and a second target sequence, respectively, in a target RNA in the biological sample, and additionally comprises a barcode region; the first and second target sequences flank a gap sequence in the target RNA; the gap sequence comprises a variant sequence; and the first probe or probe set is configured to be gap-filled, circularized, and amplified via rolling circle amplification to produce rolling circle amplification products (RCPs) containing multiple copies of a variant sequence; and b) a plurality of second probes or probe sets, wherein: each second probe or probe set hybridizes to an RCP of the RCPs and comprises an interrogatory region comprising a sequence complementary to the variant sequence. In some embodiments, the system or kit further comprises: c) one or more reagents for ligating the first probes or probe sets and/or the second probes or probe sets, and d) one or more reagents for rolling circle amplification. In some instances, the polymerase for RCA has little to no strand displacement activity. In some instances, the one or more reagents for RCA comprises a polymerase and a plurality of dNTPs. In some instances, the system or kit comprises reagents for performing in situ sequencing. In some examples, the system or kit comprises reagents for performing sequencing-by-synthesis (SBS), sequencing-by-ligation (SBL), sequencing-by-binding (SBB), or sequencing-by-avidity (SBA). In some embodiments, the system or kit comprises a library of splint oligonucleotides, wherein each splint oligonucleotide comprises ligatable ends; and a hybridization region complementary to the gap sequence in the target RNA. In some embodiments, the system or kit comprises a polymerase for performing an extension reaction using the gap sequence in the target RNA as template.

In some embodiments, the system or kit comprises a spatial array comprising an array of features on a substrate. In some instances, the array comprises a plurality of capture probes configured to capture one or more nucleic acid molecules. In some instances, a capture probe of the plurality of capture probes is configured to bind a sequence of the second probes or probe sets. In some instances, the capture probe comprises a spatial barcode corresponding to a unique spatial location of the feature on the array.

### BRIEF DESCRIPTION OF THE DRAWINGS

The drawings illustrate certain embodiments of the features and advantages of this disclosure. These embodiments are not intended to limit the scope of the appended claims in any manner.

FIG. 1 shows scenarios in detecting single-nucleotide variants using probe hybridization and ligation to discriminate among single nucleotides of interest. For instance, a padlock probe can hybridize to an RNA sequence such that an interrogatory nucleotide (X′ which is complementary to X, or Y′ which is complementary to Y) is at a ligation junction. Incorrect detection of the variant sequence can result from the high error rate (e.g., ˜5%) of a ligase when the ligation is templated on RNA. This can result in false positive detection of the variant nucleotide as well as failure to detect a true positive signal associated with the variant nucleotide.

FIG. 2 shows a method of detecting variant sequences using a first probe or probe set that is gap-filled using a target nucleic acid (e.g., RNA) as a template, generating a RCP, and using a second probe or probe set that is ligated using the RCP as a template.

FIG. 3A shows schematics of first probes or probe sets which can be gap-filled using hybridization of a splint oligonucleotide to the gap sequence followed by ligation of the splint oligonucleotide to the first probe or probe set (left panel) or polymerase extension followed by ligation of the extended probe or probe set (right panel). FIG. 3B shows a schematic of the second probe or probe set which hybridizes to the RCP and configured to be ligated by a ligase that discriminates a match with the nucleotide of interest X versus a mismatch.

FIG. 4 shows a method of detecting a variant sequence at a location in a biological sample using a first probe that is gap-filled using the target RNA as a template, followed by ligating a barcoded second probe using the RCP as a template and a ligase that discriminates a match versus a mismatch with the nucleotide of interest X at the ligation junction.

FIG. 5 shows a method of detecting variant sequences at a location in a biological sample using a first barcoded probe or probe set that is gap-filled using the target RNA as a template, followed by ligating a barcoded second probe using the RCP as a template and a ligase that discriminates a match versus a mismatch with the nucleotide of interest X at the ligation junction.

FIG. 6A and FIG. 6B show examples of detecting a nucleotide of interest in an RCP using a pair of probes which are hybridized to the RCP and are configured to be ligated at the site of the nucleotide of interest. The pair of probes can comprise a detectably labeled probe (FIG. 6A) or an intermediate probe comprising a detectable region that hybridizes to a detectably labeled probe (FIG. 6B).

FIG. 7 shows a method of detecting a nucleotide of interest in RCPs generated from first probes or probe sets using a substrate comprising capture probes for capturing ligation products of second probes or probe sets that are generated using the RCPs as ligation templates.

### DETAILED DESCRIPTION

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Single nucleotide variants (SNVs) are the most common genetic variants and ubiquitous in genomes and transcriptomes. The ability to spatially locate and quantify SNVs in cells and tissue samples has the potential to significantly propel our understanding of biology. Transcriptomic SNV detection with current technologies is challenging due to the biochemistry involved. Some in situ detection methods depend on the RNA-templated ligation of circularizable probes (e.g., padlock probes) using a ligase. However, options of RNA-templated ligases are more limited (compared to DNA-templated ligases) and in some cases, ligases used for RNA-templated ligation suffer from ligation junction biases as well as poor ability to discriminate between matched and mismatched nucleotides at the ligation junctions. These limitations may result in poor accuracy in detection of SNVs, for example, if a probe is ligated with a mismatch in the hybridization by error, and is amplified to provide many copies of a SNV that is not present in the target nucleic acid.

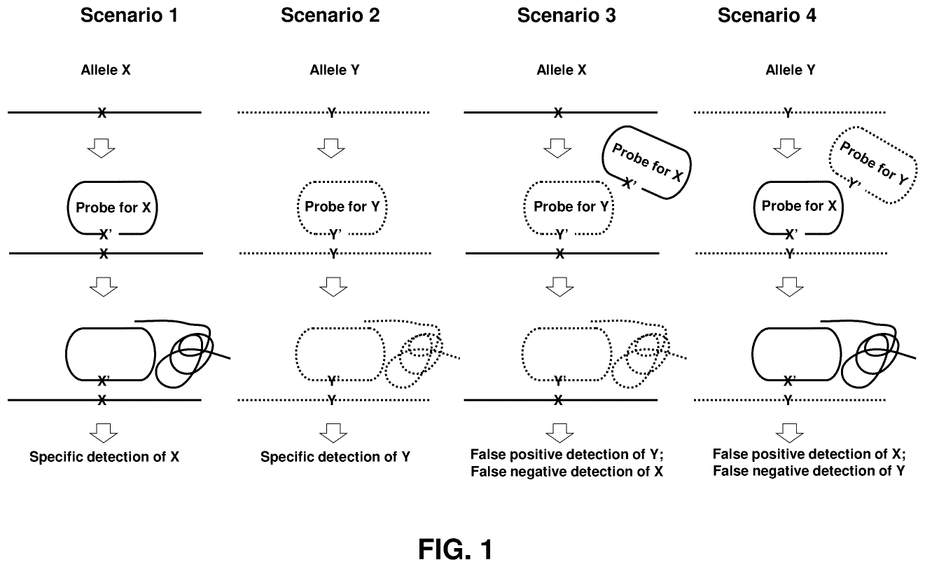

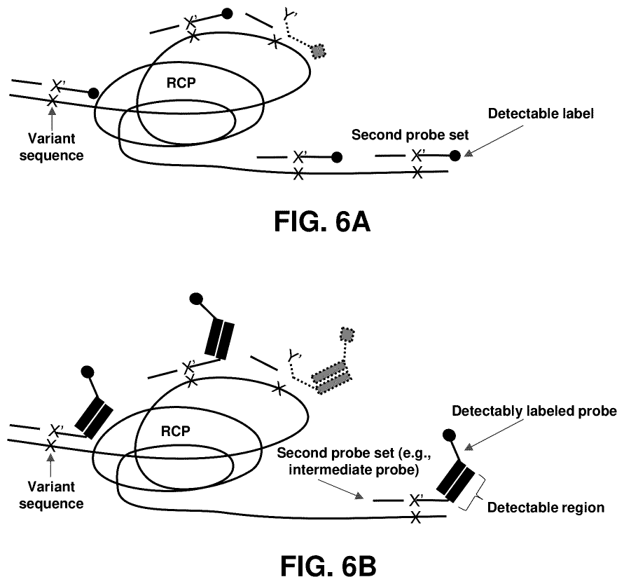

Provided herein are improved methods and compositions for detecting a single nucleotide of interest with improved accuracy using in situ or spatial array-based detection. In some embodiments, provided herein are methods and compositions that reduce errors in detecting a variant nucleotide or short sequence such as SNVs. As shown in FIG. 1, in situ SNV detection can be hampered by low ligase specificity when ligation is templated on RNA targets, where high false ligation rates (e.g., >5%) when ligating DNA probes hybridized to RNA templates can be observed. In some embodiments, provided herein is an approach to capture a short variant nucleotide or sequence with a high level of specificity by taking a gap-fill approach using a first probe or probe set that hybridizes to a target RNA, amplifying the circularized gap-filled probe by RCA, and then performing a second detection by ligating a second probe or probe set templated on the RCP, thus allowing the use of DNA-templated ligation to discriminate the variant nucleotide or short sequence which can be present in numerous copies in the RCP and in the form of DNA. In some embodiments, the ligation site in the second probe or probe set hybridized to the RCP is at the variant nucleotide or short sequence incorporated into the RCP via gap-filling and subsequent RCA. In some embodiments, a second probe or probe set comprises a detectable label (e.g., as shown in FIG. 6A) and/or a detectable region (e.g., as shown in FIG. 6B), such that the ligation product of the second probe or probe set is detected by detecting the detectable label and/or the detectable region. In some embodiments, the ligation product does not comprise a detectable label (e.g., a fluorophore conjugated to the ligation product) and comprises a detectable region which directly or indirectly binds to a detectably labeled probe. In some embodiments, the detectable region comprises a detectably labeled oligonucleotide binding sequence or a barcode region. In some embodiments, in situ detection of the RCP comprising multiple copies of the variant sequence and in situ detection of the ligation product do not comprise using a base-by-base detection method, such as sequencing-by-synthesis (SBS), sequencing-by-ligation (SBL), sequencing-by-binding (SBB), or avidity sequencing (e.g., sequencing-by-avidity (SBA)), or a combination thereof. In some embodiments, copies of the variant sequence in the RCP are not sequenced using SBS, SBL, SBB, avidity sequencing, or a combination thereof.

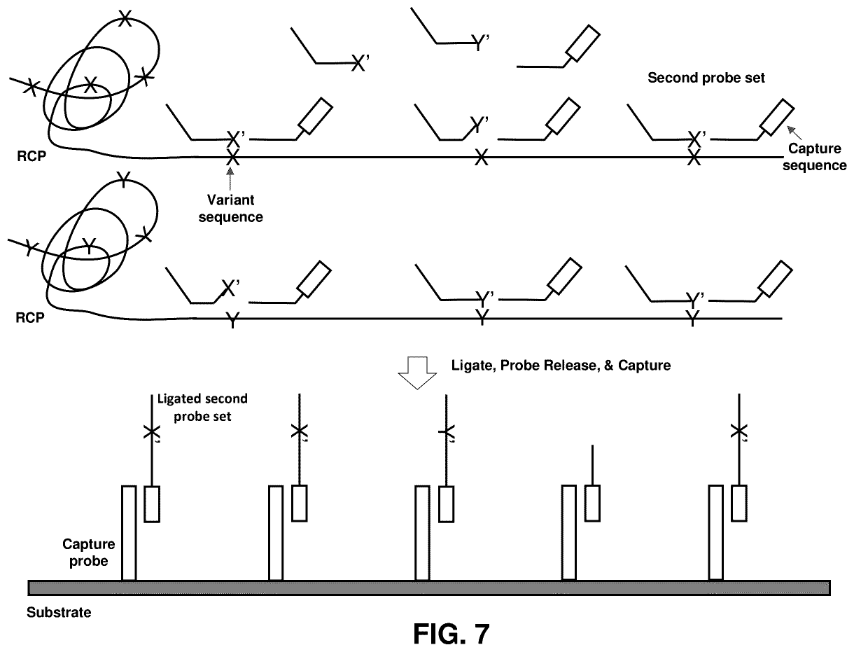

In some embodiments, the ligation product of each second probe or probe set comprises a capture sequence for capturing the ligation product on a spatial array for NGS-based detection (e.g., as shown in FIG. 7).

In some embodiments, the detection is more specific due to the increased specificity of DNA-templated DNA ligation, e.g., the ligation of the second probe or probe set. In some cases, a DNA-templated DNA ligation with a ligation error rate of no more than about 1% is used, and with the low error rate, the signals from mis-ligated probes are minimal compared to the signal from the correctly ligated probes, due to the numerous copies of variant nucleotides or sequences amplified via RCA and without the high error rates in generating the circular template for the RCA. In addition, in some instances, RCA is used to amplify a target sequence comprising a variant nucleotide or short sequence in situ and preserve the spatial location of the amplicons, in contrast to in situ PCR where diffusion of the PCR products is difficult to control.

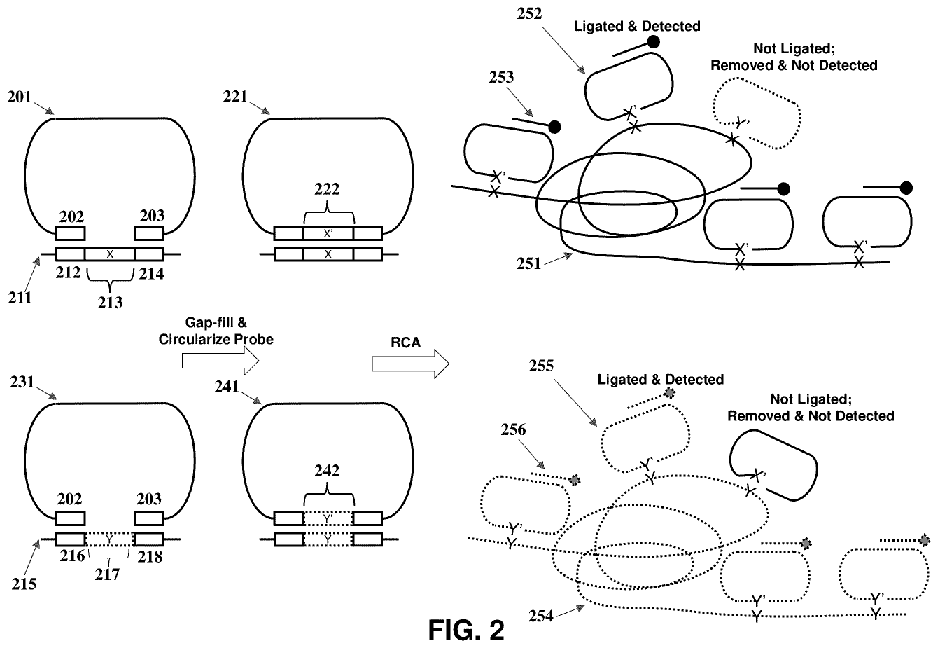

In some embodiments, provided herein are methods and compositions that reduce false positive signals (e.g., due to non-specific probe ligation) for the identification of variant sequences in cell or tissue samples (e.g., SNPs) using in situ assays or spatial-array based assays. In some embodiments, a method of SNV detection disclosed herein uses a first probe or probe set which is a gap-fill probe or probe set, with the gap over the SNV site or a hotspot region containing the SNV site. In some embodiments, the gap in the first probe or probe set is filled with an oligonucleotide (e.g., a splint oligonucleotide) or through polymerization using a reverse transcriptase and the first probe or probe set is circularized. In some embodiments, rolling circle amplification is performed on the circularized gap-filled probe or probe set, generating an RCP containing multiple copies of the SNV (or a hotspot region containing the SNV) and the RCPs corresponding to different SNVs can be detected, as shown in FIG. 2.

In FIG. 2, the biological sample is contacted with a first probe or probe set 201 comprising a first probe region 202 and a second probe region 203 (e.g., 5′ and 3′ arms of a padlock probe) that hybridize to a first target sequence 212 and a second target sequence 214, respectively, in a target nucleic acid 211 (e.g., an RNA or cDNA) in the biological sample. In the target nucleic acid 211, the first target sequence 212 and a second target sequence 214 flank a gap sequence 213 comprising a variant sequence X. An additional first probe or probe set 231 comprising a first probe region 202 and a second probe region 203 (e.g., 5′ and 3′ arms of a padlock probe) hybridizes to a first target sequence 216 and a second target sequence 218, respectively, in an additional target nucleic acid 215 (e.g., an RNA or cDNA) in the biological sample. In the additional target nucleic acid 215, the first target sequence 216 and a second target sequence 218 flank a gap sequence 217 comprising a variant sequence Y. In some instances, two different target nucleic acids 211 and 215 comprise different variant sequences 213 and 217 but share common sequences that flank the variant sequences. In some embodiments, the first and second probe regions of the first probes or probe sets are common among a plurality of first probes or probe sets that target a plurality of target nucleic acids that comprise different variant sequences (e.g., in the target nucleic acid 211 and additional target nucleic acid 215 of FIG. 2). In some instances, the first probe or probe set 201 and additional first probe or probe set 231 are gap-filled with sequences 222 and 242, respectively, to generate circularized gap-filled probes or probe sets 221 and 241. In some instances, the circularized gap-filled probes or probe sets 221 and 241 are used as template for rolling circle amplification (RCA) and products 251 and 254 are generated. In FIG. 2, a second probe or probe set 252 hybridizes to an amplification product 251 of the circularized gap-filled probe 221 generated from the first probe or probe set 201. In FIG. 2, an additional second probe or probe set 255 hybridizes to an amplification product 254 of the circularized gap-filled probe 241 generated from the first probe or probe set 231. Detectably labeled probes 253 and 256 are used to detect sequences in the second probe or probe sets. In some instances, second probes or probe sets that are not ligated due to a mismatch with the variant sequence in the RCP are not detected.

In some embodiments, a method disclosed herein comprises decoding a first barcode region in the first probe or probe set and/or a second barcode region in the second probe or probe set. In some embodiments, a first barcode region comprising one or more barcode sequences associated with the first probe or probe set is decoded to identify the target nucleic acid (e.g., transcripts from KRAS versus TP50) and a second barcode region comprising one or more barcode sequences associated with the second probe or probe set is decoded to identify the variant sequence (e.g., a SNP or point mutation), with different barcode sequences associated with each possible nucleotide at a nucleotide position. In some embodiments, although not necessary, ligation of the second probe or probe set is performed using a ligase having a high specificity in DNA-templated ligation (e.g., ligation of DNA probe molecules templated on an RCP which is DNA). In some embodiments, a ligase having a relatively low specificity is used in the RCP-templated ligation, since the RCP contains multiple copies of the SNV such that the incorrectly ligated second probes or probe set are still in a minority.

In some embodiments, detection of the multiple copies of the SNV (or a hotspot region containing the SNV) in the RCP comprises using a second probe or probe set (e.g., a padlock probe) that hybridizes to the RCP. In some embodiments, the second probe or probe set hybridization enables the use of a ligase (e.g., a DNA-templated DNA ligase) that performs ligation on DNA templates with higher fidelity and better ability to discriminate single nucleotide differences than ligases performing RNA-templated ligation. In some embodiments, even with a ligase having a relatively low specificity, the overall specificity of the detection may not be compromised (or may be less compromised than using a low-specificity ligase in RNA-templated ligation) because the RCP contains multiple copies of the SNV, any mis-ligated second probes or probe sets would be in a minority compared to the correctly ligated second probes or probe sets.

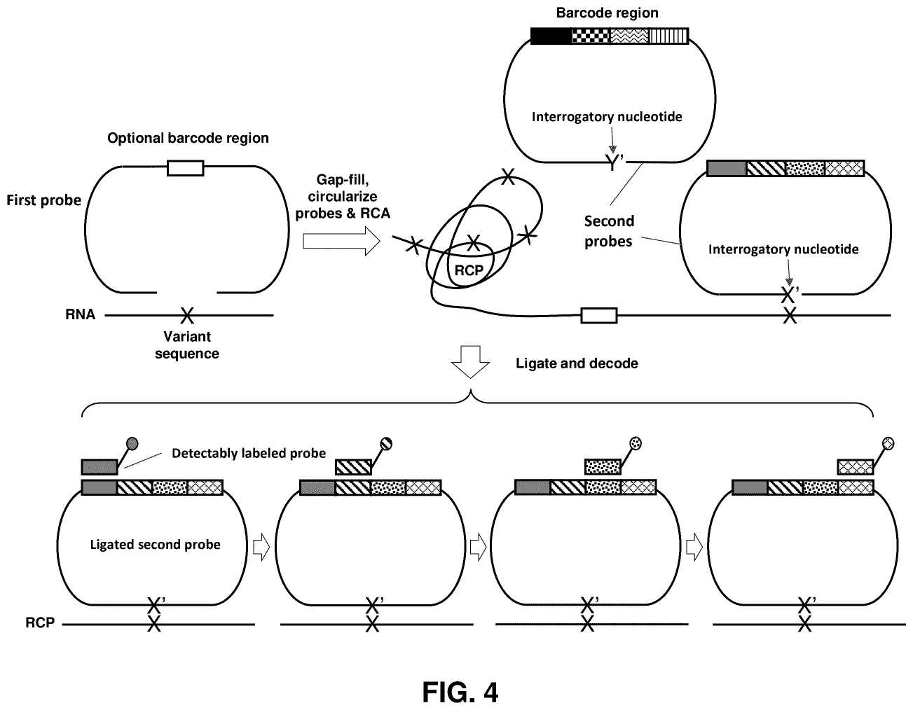

In some embodiments, the second probe or probe set comprises a barcode region with different barcode sequences for different variant sequences in a target nucleic acid, such as a barcode region corresponding a wildtype SNV and a different barcode region corresponding to a mutant SNV, enabling decoding of the transcripts in a sample to determine the presence or absence of the mutant SNV (e.g., as shown in FIG. 4).

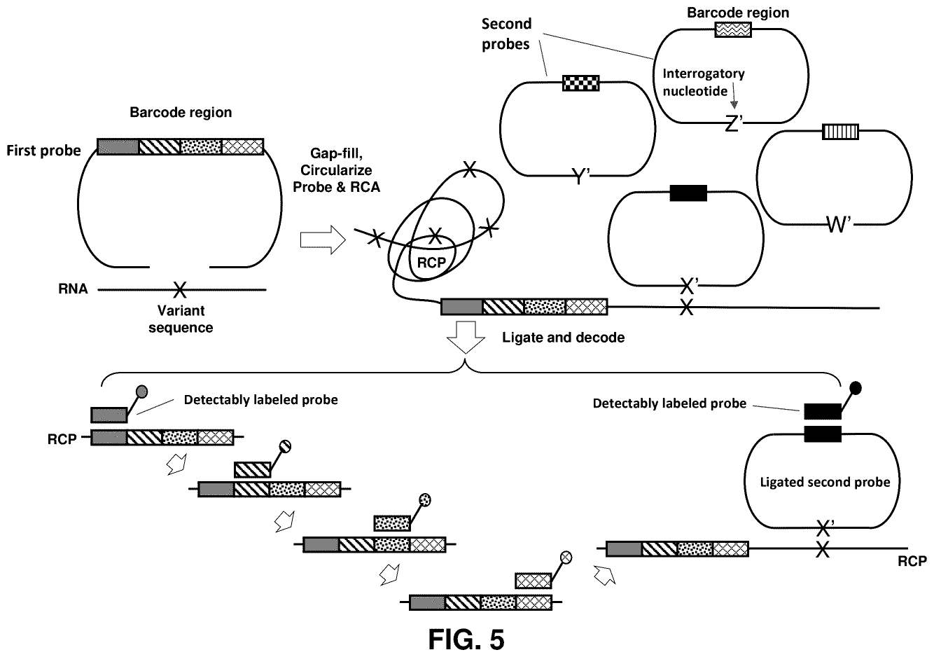

In some embodiments, the first probe or probe set comprises a first barcode region corresponding to the identity of a target nucleic acid and the RCP comprises multiple copies of a sequence complementary to the barcode region. In some embodiments, the RCPs in a sample is detected to decode the barcode regions (complementary to be first barcode region) in the RCPs, thereby identifying the target nucleic acid at locations in the sample (e.g., both wildtype and mutant KRAS can be detected by decoding the first barcode regions). In some embodiments, in addition to the probe hybridization and detection cycles to decode the barcode region in the RCP, the detection of an SNV (e.g., a mutant KRAS versus wildtype KRAS) comprises performing one additional decoding cycle to detect a second barcode region in a second probe or probe set that is hybridized to the RCP at the SNV site and ligated by a ligase that discriminates a particular variant nucleotide from one or more other variant nucleotides and the SNV site (e.g., as shown in FIG. 5). In some embodiments, one or more barcode sequences in the second barcode region can be associated with a target nucleic acid (e.g., KRAS versus TP50). In some embodiments, a plurality of different second probes or probe sets are used, where each different second probe or probe set is configured to interrogate a possible nucleotide at the SNV position and comprises a different barcode region for the corresponding nucleotide. In some embodiments, one or more barcode sequences in the second barcode region are not specific for any particular target nucleic acid (e.g., KRAS versus TP50) but are associated with a possible nucleotide (e.g., A, T/U, C, or G) at the SNV position. For example, the same barcode sequence is included in a second probe or probe set for detecting an RCP containing an SNV of a first gene (e.g., “C” at a position in KRAS) as well as a second probe or probe set for detecting an RCP containing an SNV of a second gene (e.g., “C” at a position in TP50). In some embodiments, the second probe or probe set comprises a single barcode region corresponding to a particular variant nucleotide (e.g., A, T/U, C, or G).

In some embodiments, a method disclosed herein does not rely on the discrimination of a variant sequence during the hybridization of the first probe or probe set to target RNAs, or the discrimination of the variant sequence during RNA-templated ligation of the first probe or probe set. In some embodiments, the method comprises variant sequence detection and identification (e.g., genotyping) in a readout stage, e.g., after the first probe or probe set hybridization to a target RNA, gap-filling and circularization, and generation of RCPs. In some embodiments, the method comprises using hybridization of a second probe or probe set to an RCP and generation of a ligation product of the second probe or probe set using the RCP as a template. In some embodiments, the second probe is a circularizable probe such as a padlock probe (e.g., as shown in FIG. 4 or FIG. 5). In some embodiments, the second probe set comprises a pair of probe molecules which are configured to hybridize to the RCP and the second probe set comprises an interrogatory nucleotide for the SNV. In some embodiments, the second probe set is detectably labeled (e.g., as shown in FIG. 6A). In some embodiments, the second probe set comprises a pair of probe molecules which are configured to hybridize to the RCP and the second probe set comprises a detectable region configured to hybridize to a detectably labeled probe (e.g., as shown in FIG. 6B). In some embodiments, the detectable region comprises a barcode region comprising one or more barcode sequences, and one or more detectably labeled probes complementary to the barcode sequence(s) are used to decode the barcode region (e.g., as shown in FIG. 4 and FIG. 5, where the circularizable second probes can be replaced by pairs of probe molecules that are ligated to form linear probes comprising one or more barcode sequences in their 3′ overhangs and/or one or more barcode sequences in their 5′ overhangs). In some embodiments, the readout comprises identifying the sequence variations using sequential hybridization of probes (e.g., detectably labeled probes, or intermediate probes configured to directly or indirectly bind to detectably labeled probes) to the RCP (e.g., at a barcode sequence associated with the first probe or probe set) and/or to the ligation product of the second probe or probe set (e.g., at a barcode sequence associated with the second probe or probe set).

In some embodiments, the readout comprises capturing the ligation product or a complement thereof on an array of capture probes affixed to a substrate, such that the spatial information from a sample (e.g., a cell or tissue sample) is preserved in the array (e.g., as shown in FIG. 7). In some embodiments, a capture probe on the array comprises a spatial barcode corresponding to the spatial location of the capture probe in the array. In some embodiments, the readout comprises the generation and sequencing of a spatially labeled polynucleotide that comprises a sequence of the ligation product (or a complement thereof) and a sequence of a spatial barcode (or a complement thereof). In some embodiments, sequencing the spatially labeled polynucleotide comprises using a high throughput nucleic acid sequencing technique. In some embodiments, sequencing the spatially labeled polynucleotide can comprise sequencing-by-synthesis (SBS), sequencing-by-ligation (SBL), sequencing-by-binding (SBB), or avidity sequencing, or a combination thereof.

### II. Nucleic Acid Analytes and Target Sequences

In some aspects, provided herein are methods and compositions for analysis of target nucleic acids. In some embodiments, the target nucleic acids comprise RNA. In some embodiments, the target nucleic acids comprise genomic DNA. In some embodiments, the target nucleic acids comprise cDNA. In some embodiments, one or more target nucleic acids each comprises a variant sequence of one or more nucleotides. In some embodiments, one or more target nucleic acids each comprises a single-nucleotide polymorphism (SNP). In some embodiments, one or more target nucleic acids each comprises a single-nucleotide variant (SNV). In some embodiments, one or more target nucleic acids each comprises a single-nucleotide substitution. In some embodiments, one or more target nucleic acids each comprises a point mutation. In some embodiments, one or more target nucleic acids each comprises a single-nucleotide insertion. In some embodiments, one or more target nucleic acids each comprises a single-nucleotide deletion. In any of the embodiments herein, target genomic DNA, target RNA, and/or target cDNA comprising one or more sequence variants at one or more genomic loci is analyzed as described herein. In some embodiments, target genomic DNA, target RNA, and/or target cDNA comprising one or more single-nucleotide differences (e.g., SNPs, SNVs, point mutations, etc.) at one or more genomic loci are analyzed, and the identity of one or more single-nucleotide differences is determined in situ in a sample (e.g., as described in Section V) or using a spatial assay (e.g., as described in Section VI).

In some embodiments, the target nucleic acid comprises a nucleotide variation, a nucleotide polymorphism, a mutation, a substitution, an insertion, a deletion, a translocation, a duplication, an inversion, and/or a repetitive sequence, in a variant sequence among a plurality of different sequences to be identified in situ in a biological sample or using a spatial assay. In some embodiments, the variant sequence is a single nucleotide, for instance, a single nucleotide variation (SNV), a single nucleotide polymorphism (SNP), a point mutation, a single nucleotide substitution, a single nucleotide insertion, or a single nucleotide deletion. In some embodiments, the variant sequence comprises multiple nucleotides, and each nucleotide is independently at the position of an SNV, an SNP, a point mutation, a single nucleotide substitution, a single nucleotide insertion, or a single nucleotide deletion. In some embodiments, the target nucleic acid is an RNA, such as an miRNA or a transcript of an oncogene, a tumor suppressor gene, an immune gene, or an antigen receptor gene.

The methods, probes, systems and kits disclosed herein can be used to detect and analyze a wide variety of different analytes. In some embodiments, analytes are derived from a specific type of cell and/or a specific sub-cellular region. For example, analytes are derived from cytosol, from cell nuclei, from mitochondria, from microsomes, and more generally, from any other compartment, organelle, or portion of a cell. In some embodiments, permeabilizing agents that specifically target certain cell compartments and organelles are used to allow access of one or more reagents (e.g., probes for analyte detection) to the analytes in the cell or cell compartment or organelle.

In some embodiments, analytes of particular interest include nucleic acid molecules (e.g., cellular nucleic acids), such as DNA (e.g. genomic DNA, cDNA, mitochondrial DNA, plastid DNA, viral DNA, etc.) and RNA (e.g. mRNA, microRNA, rRNA, snRNA, viral RNA, etc.), and synthetic and/or modified nucleic acid molecules (e.g. including nucleic acid domains comprising or consisting of synthetic or modified nucleotides such as LNA, PNA, morpholino, etc.).

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. In some embodiments, the RNA analyte is a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). In some embodiments, the RNA is double-stranded RNA or single-stranded RNA. In some embodiments, the RNA is circular RNA. In some embodiments, the RNA is a bacterial rRNA (e.g., 16s rRNA or 23s rRNA).

In some embodiments described herein, an analyte is a denatured nucleic acid, wherein the resulting denatured nucleic acid is single-stranded. In some embodiments, the nucleic acid may be denatured, for example, optionally using formamide, heat, or both formamide and heat. In some embodiments, the nucleic acid is not denatured for use in a method disclosed herein.

Methods, probes, and kits disclosed herein can be used to analyze any number of analytes. In some embodiments, the number of analytes that are analyzed is at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample. In some instances, different analytes are analyzed within an individual feature of the substrate.

In any embodiment described herein, the analyte comprises or is associated with a target sequence. In some embodiments, the target nucleic acid and the target sequence therein is endogenous to the sample, generated in the sample, added to the sample, or associated with an analyte in the sample. In some embodiments, the target nucleic acid is an endogenous RNA in the sample (e.g., cell or tissue sample). In some embodiments, the target sequence is a single-stranded target sequence (e.g., a sequence in a rolling circle amplification product). In some embodiments, the target sequence is a single-stranded target sequence (e.g., in a probe bound directly or indirectly to the analyte). In some embodiments, the target sequence is a single-stranded target sequence in a primary probe that binds to an analyte of interest in the biological sample. In some embodiments, the target sequence is a single-stranded target sequence in an intermediate probe which directly or indirectly binds to a primary probe or product thereof, where the primary probe binds to an analyte of interest in the biological sample. In some embodiments, the target sequence is a single-stranded target sequence in a secondary probe that binds to the primary probe or product thereof. In some embodiments, the analytes comprise one or more single-stranded target sequences.

In some embodiments, provided herein are methods for analysis of a target nucleic acid using different probes (e.g., splint oligonucleotides) to compete for hybridization to a target nucleic acid. In some embodiments, the splint oligonucleotides are ligated to a first probe or probe set disclosed herein to circularize the first probe or probe set. In some embodiments, provided herein are methods for analysis of a target nucleic acid using primer extension to gap-fill a first probe or probe set upon its hybridization to a target nucleic acid, and the gap-filled first probe or probe set is circularized by ligation. In some embodiments, the circularized probes are amplified (e.g., using RCA) and the amplicons are detected in situ. In some embodiments, a probe or probe set (e.g., a second probe or probe set described herein) is hybridized to an amplicon (e.g., an RCP) at a site corresponding to a target sequence and ligated in a manner that discriminates a variant sequence versus one or more other variant sequences in the target sequence. In some embodiments, the amplicons (of the first probe or probe set) and/or the ligation products of the second probes or probe sets targeting various target nucleic acids and variant sequences therein are detected in situ, e.g., using sequential probe hybridization to barcode sequences in the amplicons and/or barcode sequences in the ligation products. In some embodiments, the ligation products or portions thereof (or complements of the ligation products or portions thereof) are captured on a spatial array for subsequent processing and analysis.

In some embodiments, probes or probe sets that target common regions adjacent to hotspots for mutation are used. In some embodiments, the common regions flank a gap sequence in the target nucleic acid. In some embodiments, a circularizable probe (e.g., a padlock probe) or probe set (e.g., a pair of split probes that is configured to be ligated to form a circularizable padlock probe) comprises a first target binding region at the 5′ end and a second target binding region at the 3′ end, and the first and second binding regions hybridize to sequences flanking a targeted variant nucleotide or short sequence (e.g., an SNP or SNV) in a target RNA. In some embodiments, the circularizable probe is a padlock probe comprising a first target binding region at the 5′ end and a second target binding region at the 3′ end. In some embodiments, the circularizable probe set is a split probe set that is configured to be ligated to form a padlock probe. In some embodiments, the split probe set is an asymmetric split probe pair, where one probe is longer or shorter than the other probe. In some embodiments, in the asymmetric split probe pair, the target binding region of one probe is longer or shorter than the target binding region of the other probe. In some embodiments, in the asymmetric split probe pair, each probe comprises a splint binding region complementary to a portion of a splint, and the splint binding region of one probe is longer or shorter than the splint binding region of the other probe. In some cases, the circularizable probe can bind different target RNAs comprising different variant nucleotides or short sequences (e.g., an SNP or SNV). In some embodiments, a probe set comprises a first probe molecule and a second probe molecule flanking a targeted variant nucleotide or short sequence (e.g., an SNP or SNV) in a target RNA. In some cases, the probe set comprises binding sequences that are complementary to different target RNAs comprising different variant nucleotides or short sequences (e.g., an SNP or SNV). In some embodiments, the gap sequence comprises one or more hotspots for mutation. In some embodiments, the gap sequence comprises a variant sequence among a plurality of different variant sequences. In some embodiments, gaps in the first probes or probe sets upon hybridization to their nucleic acid targets are filled by polymerization. In some embodiments, the gaps are filled by splint ligation, using a library of splint oligonucleotides that are diverse in sequences and comprise a plurality of possible variant sequences (e.g., possible mutations for the hotspots). In some embodiments, the library of splint oligonucleotides is incubated with the sample for hybridization to target nucleic acid molecules, allowing the best matching splint oligonucleotide to outcompete other splint oligonucleotides in the library. In some embodiments, after washing the sample, the best matching splint oligonucleotides are ligated to the first probes or probe sets to generate circularized gap-filled probes, and the circularized gap-filled probes are amplified.

In some embodiments, the incorporation of variant sequence information into a first probe or probe set disclosed herein is hypothesis-free, requiring no prior knowledge of whether and which particular variant sequences are present in target nucleic acid molecules in a sample. In some embodiments, the gaps in the first probes or probe sets hybridized to target nucleic acid molecules are filled using polymerization (e.g., reverse transcription) templated on the gap sequences, followed by circularization of the extended first probes or probe sets (e.g., using a ligase having RNA-templated ligase activity).

In some embodiments, the incorporation of variant sequence information into the probes can take into consideration possible variant sequences covered by the gap sequences. In some embodiments, at least some of the gaps are filled by ligating a library of splint oligonucleotides comprising complementary sequences to the possible variant sequences with the first probes or probe sets.

### III. Gap-Filled Probes and Probe Circularization

In some embodiments, a first probe or probe set is designed to hybridize to a target nucleic acid (e.g., a target RNA) and flank a variant nucleotide or short sequence (such as an SNV or a mutation hotspot), leaving a gap of one or more nucleotides. In some embodiments, a gap is filled by extending the 3′ end of a first probe which is a gap-fill padlock probe. In some embodiments, filling a gap comprises extending the 3′ end of the first probe over the targeted variant nucleotide or short sequence (e.g., an SNP or SNV), incorporating the sequence information of the correct nucleotide(s) in the form of complementary nucleotide(s) in the gap-filled probe, and ligating the gap-filled probe to generate a circularized gap-filled probe. In some embodiments, the circularized gap-filled probe is amplified, producing an RCA product containing a plurality of unit sequences each comprising a copy of the variant nucleotide or short sequence. In some embodiments, the first probe or probe set comprise exogenous molecules added to the biological sample. In some embodiments, the first probe or probe set hybridizes to a target RNA in the biological sample (e.g., the cell or tissue sample).

In some embodiments, provided herein are probe molecules that are circularized after hybridization to a target nucleic acid and gap-filling using the target nucleic acid as a template. In some embodiments, the template for the gap-fill of the first probe or probe set is an RNA. In some embodiments, a first probe or probe set is hybridized to a target nucleic acid molecule comprising a gap sequence which comprises one of multiple variant sequences, and the first probe or probe set is gap-filled and then circularized to generate a circularized gap-filled probe comprising a gap-filled sequence complementary to the gap sequence in the target nucleic acid molecule. The circularized gap-filled probe comprising at least portions of the complement of the gap sequence is amplified (e.g., through RCA) and the amplification product is detected in order to detect the variant sequence in the target nucleic acid molecule.

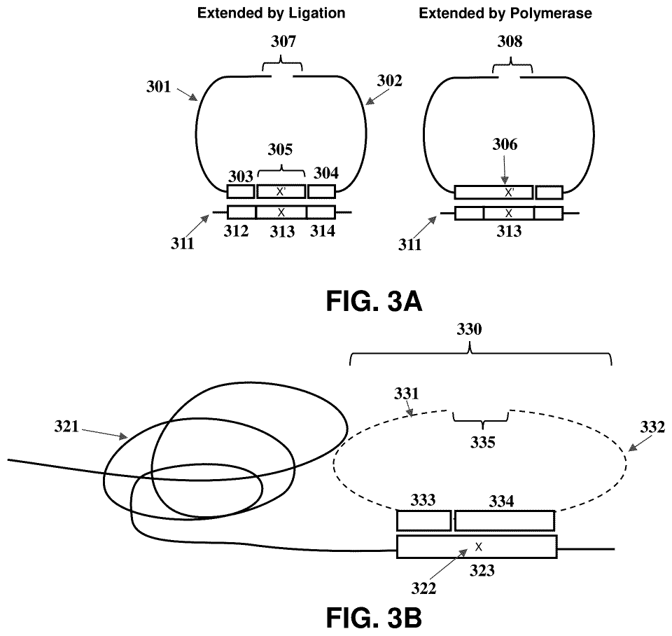

As shown in the left panel of FIG. 3A, a first probe or probe set comprises a first probe molecule 301 and a second probe molecule 302. In some cases, the first probe molecule 301 comprises a first probe region 303 hybridized to a first target sequence 312 in the target nucleic acid molecule 311. In some cases, the second probe molecule 302 comprises a second probe region 304 hybridized to a second target sequence 314 in the target nucleic acid molecule 311. The target nucleic acid comprises a gap sequence 313 comprising one of multiple variant sequences (indicated by X). A splint 305 comprising a sequence complementary to the gap sequence 313 is hybridized to the target nucleic acid molecule 311 and ligated to the first probe molecule 301 and second probe molecule 302 to fill the gap of the first probe set. In some embodiments, the first probe or probe set is gap-filled and circularized using the splint 305 to generate a circularized gap-filled probe comprising a gap-filled sequence 305 complementary to the gap sequence 313 in the target nucleic acid molecule. In some embodiments, a second ligation 307 is performed to generate a circularized gap-filled probe or probe set. As shown in the right panel of FIG. 3A, in some embodiments, a first probe or probe set is be circularized by using polymerase. In some embodiments, the first probe or probe set is gap-filled and circularized by extending the sequence 303 to generate a circularized gap-filled probe comprising a gap-filled sequence 306 complementary to the gap sequence 313 in the target nucleic acid molecule.

In some embodiments, provided herein is a method for analyzing a biological sample, the method comprising contacting the biological sample with a first probe or probe set comprising a first probe region and a second probe region (e.g., 5′ and 3′ arms of a padlock probe) that hybridize to a first target sequence and a second target sequence, respectively, in a target nucleic acid (e.g., an RNA or cDNA) in the biological sample. In some embodiments, the first and second probe regions of the first probes or probe sets are common among a plurality of first probes or probe sets that target a plurality of target nucleic acids that comprise different variant sequences. In some embodiments, each of the plurality of target nucleic acids comprises a common first target sequence (among the plurality of target nucleic acids) and a common second target sequence (among the plurality of target nucleic acids) that are complementary to the common first and second probe regions, respectively, among the plurality of first probes or probe sets. In some embodiments, the plurality of first probes or probe sets comprise molecules of the same nucleic acid sequence. In some embodiments, the plurality of first probes or probe sets comprise molecules of different nucleic acid sequences. In some embodiments, any two or more different nucleic acid sequences of the first probes or probe sets comprise i) the common first and second probe regions, and ii) different backbone sequences that are not complementary to target nucleic acid sequences. In some embodiments, the different backbone sequences each comprise a different barcode region. In some embodiments, the barcode region identifies one or more first probes or probe sets from other first probes or probe sets. In some embodiments, the barcode sequence is associated with, corresponds to, and/or identifies a target nucleic acid or a sequence therein. In some embodiments, the barcode sequence is associated with, corresponds to, and/or identifies the plurality of target nucleic acids that comprise different variant sequences (e.g., a barcode sequence can correspond to target nucleic acids of a gene comprising various SNPs and/or point mutations in the gene).

In some embodiments, the first and second target sequences are separated by a gap sequence in the target nucleic acid. In some embodiments, the gap sequence is about or at least 4, about or at least 6, about or at least 8, about or at least 10, about or at least 12, about or at least 14, about or at least 16, about or at least 18, about or at least 20, or more nucleotides in length. In some embodiments, the gap sequence comprises a variant sequence among a plurality of different variant sequences. In some embodiments, the plurality of first probes or probe sets that target a plurality of target nucleic acids that comprise different variant sequences do not hybridize to the gap sequences (which comprise the different variant sequences), and instead hybridize to common first and second target sequences that flank the gap sequences.

In some embodiments, upon hybridization to the target nucleic acid, the first probe is circularized to generate a circularized gap-filled probe comprising a gap-filled region complementary to the gap sequence. In some embodiments, the gap-filled region is generated using gap-filling by polymerization, or gap-fill splint ligation, or a combination thereof. In some embodiments, an RCP of the circularized gap-filled probe is generated in the biological sample, and the RCP comprises multiple copies of the gap sequence.

In some embodiments, the first probe or probe set comprises a 5′ region and a 3′ region that hybridize to sequences adjacent to a hotspot for mutation in the target nucleic acid. In some embodiments, upon hybridization of a first probe or probe set to the target nucleic acid molecule, the 3′ terminal nucleotide and the 5′ terminal nucleotide of the first probe or probe set are not juxtaposed directly next to each other; as such, a ligase alone cannot catalyze the formation of a phosphodiester bond directly between the 5′ phosphate of the 5′ terminal nucleotide and the 3′ hydroxyl of the 3′ terminal nucleotide. In some embodiments, upon hybridization of a first probe or probe set to the target nucleic acid molecule, the 3′ terminal nucleotide and the 5′ terminal nucleotide of the first probe or probe set are separated from each other by a gap of between about 1 and about 40 nucleotides in length. In some embodiments, the gap is about 2, about 5, about 10, about 15, about 20, about 25, about 30, about 35, about 40, about 45, about 50, or of any integer (or range of integers) of nucleotides in between the indicated values. In some embodiments, the gap is no more than about 40 nucleotides in length. In some embodiments, the gap is no more than about 30 nucleotides in length. In some embodiments, the gap is no more than about 20 nucleotides in length. In some embodiments, the gap is no more than about 10 nucleotides in length. In some embodiments, the gap is about 6, about 8, about 10, about 12, about 14, about 16, about 18, about 20, about 22, about 24, or of any integer (or range of integers) of nucleotides in between the indicated values. In some embodiments, the gap is no more than about 5 nucleotides in length. In some embodiments, the gap is about 5 nucleotides in length. In some embodiments, the gap is a single nucleotide.

In some embodiments, a first probe or probe set disclosed herein does not comprise any nucleic acid barcode sequence. In some embodiments, the first probes or probe sets for hybridizing to multiple different target nucleic acids comprises a common sequence that is not complementary to the target nucleic acids. For instance, the backbone sequences of a plurality of first probes or probe sets for detecting different variant sequences of a target nucleic acid comprises a common backbone sequence. In other examples, the backbone sequences of a plurality of first probes or probe sets for detecting different target nucleic acids comprises a common backbone sequence, and the arms of the gap-fill padlock probes can be different such that they can specifically hybridize to the target nucleic acids. In some embodiments, the backbone sequences of the plurality of first probes or probe sets do not contain any nucleic acid barcode sequence that uniquely corresponds to a particular target nucleic acid or a particular sequence variant thereof. In some embodiments, the gap-filled sequence is detected downstream, e.g., as described in Section V.

In some embodiments, a first probe or probe set disclosed herein comprises one or more barcode regions. In some embodiments, each barcode region independently comprises one or more barcode sequences. The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the number of distinct barcode sequences in a population of first probes or probe sets is less than the number of distinct targets of the first probes or probe sets, and yet the distinct targets may still be uniquely identified from one another, e.g., by encoding a probe with a different combination of barcode sequences. However, not all possible combinations of a given set of barcode sequences need be used. For instance, each probe may contain 1, 2, 3, 4, 5, 6, 7, 8, 9, 10, 11, 12, 13, 14, 15, etc. or more barcode sequences. In some embodiments, a population of first probes or probe sets each contain the same number of barcode sequences, although in other cases, there may be different numbers of barcode sequences present on the various probes or probe sets. In some embodiments, the barcode sequences or any subset thereof in the population of first probes or probe sets are independently and/or combinatorially detected and/or decoded.

### A. Gap-Fill Polymerization

In some embodiments, a gap in a first probe or probe set hybridized to the target nucleic acid molecule is filled by extending a 3′ end of the first probe or probe set. In some embodiments, a polymerase is used to extend the 3′ end using the target nucleic acid molecule as a template, thereby filling the gap using the nucleotide sequence in the target nucleic acid molecule. In some embodiments, gap filling by the polymerase incorporates nucleotides residues into the first probe or probe set, and the incorporated nucleotide sequence is complementary to the gap sequence or a portion thereof in the target nucleic acid molecule.

In some instances, the gap filling is performed using a polymerase (e.g., DNA polymerase) in the presence of appropriate dNTPs and other cofactors, under isothermal conditions or non-isothermal conditions. Exemplary DNA polymerases include but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

In some instances, the gap filling is performed using a DNA polymerase capable of incorporating at least about 25, at least about 50, at least about 100, at least about 125, at least about 150, at least about 175, at least about 200, at least about 225, at least about 250, at least about 300, at least about 400, at least about 500, at least about 600, or at least about 1,000 nucleotides in a single binding event before dissociating from the target nucleic acid molecule.

Incorporation of the correct nucleotides to a growing strand of DNA, as determined by the template, is known as sequence fidelity. In some embodiments, a high fidelity DNA polymerase is used for gap filling and examples include but are not limited to: Taq DNA polymerase, Phusion® High-Fidelity DNA Polymerase, KAPA Taq, KAPA Taq HotStart DNA Polymerase, KAPA HiFi, and/or Q5® High-Fidelity DNA Polymerase.

In some instances, the gap filling is performed using a polymerase having no or limited strand displacement activity, such that an extended 3′ region of the first probe or probe set does not displace the 5′ region hybridized to the nucleic acid molecule. For example, T4 and T7 DNA Polymerases lack strand displacement activity and can be used for this purpose. In some embodiments, especially where the target nucleic acid is RNA, the polymerase is a reverse transcriptase. Reverse transcriptases having reduced strand displacement activity can be used, see, e.g., Martin-Alonso et al., ACS Infect. Dis. 2020, 6, 5, 1140-1153, which is incorporated herein by reference in its entirety.

In some embodiments, the 3′ region of the first probe or probe set extended by the polymerase is juxtaposed to the 5′ region of the first probe or probe set, forming a nick. In some embodiments, the ligation involves template dependent ligation, e.g., using the gap sequence in the target nucleic acid as template. In some embodiments, the ligation involves template independent ligation. The nick can be ligated using chemical ligation. In some embodiments, the chemical ligation involves click chemistry.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. In some aspects, the ligase used herein is a DNA ligase. In some aspects, the ligase used herein is an ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9°N) DNA ligase (9°N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a SplintR® ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity. In some embodiments, the ligase is a ssDNA ligase. In some embodiments, the ssDNA ligase is a bacteriophage TS2126 RNA ligase or an archaebacterium RNA ligase or a variant or derivative thereof. In some embodiments, the ligase is Methanobacterium thermoautotrophicum RNA ligase 1, CircLigase™ I, CircLigase™ II, T4 RNA ligase 1, or T4 RNA ligase 2, or a variant or derivative thereof.

### B. Gap-fill Oligonucleotide Hybridization

In some embodiments, a gap in a first probe or probe set hybridized to the target nucleic acid molecule is filled by a splint oligonucleotide. In some embodiments, the splint oligonucleotide is ligated to the first probe at the 3′ and 5′ ends of the splint oligonucleotide using the target nucleic acid molecule as a ligation template.

In some embodiments, the splint oligonucleotide comprises a sequence complementary to a nucleotide variation, a nucleotide polymorphism, a mutation, a substitution, an insertion, a deletion, a translocation, a duplication, an inversion, and/or a repetitive sequence, for identifying a variant sequence among a plurality of different sequences in situ in a biological sample. In some embodiments, the splint oligonucleotide comprises a sequence complementary to a single nucleotide, for instance, a single nucleotide variation (SNV), a single nucleotide polymorphism (SNP), a point mutation, a single nucleotide substitution, a single nucleotide insertion, or a single nucleotide deletion. In some embodiments, the splint oligonucleotide comprises a sequence complementary to a sequence comprising multiple nucleotides, and each nucleotide is independently at the position of an SNV, an SNP, a point mutation, a single nucleotide substitution, a single nucleotide insertion, or a single nucleotide deletion.

In some embodiments, provided herein is a library of splint oligonucleotides comprising i) a splint oligonucleotide comprising a sequence complementary to a nucleotide variation, a nucleotide polymorphism, a mutation, a substitution, an insertion, a deletion, a translocation, a duplication, an inversion, and/or a repetitive sequence, and ii) another splint oligonucleotide which does not comprise a sequence complementary to the nucleotide variation, nucleotide polymorphism, mutation, substitution, insertion, deletion, translocation, duplication, inversion, and/or repetitive sequence. In some embodiments, the library of splint oligonucleotides can comprise i) a splint oligonucleotide comprising a sequence complementary to a variant sequence or deletion or insertion, and ii) another splint oligonucleotide which does not comprise a sequence complementary to the variant sequence or deletion or insertion. For example, wildtype and variant splint oligonucleotides in the library, when contacted with the biological sample, compete with one another for hybridization to a gap sequence comprising a variant sequence, and the complementary variant splint oligonucleotide can outcompete the wildtype splint oligonucleotide which is not complementary to the variant sequence (e.g., one or more nucleotides) in the gap sequence. The competition among splint oligonucleotides can allow the use of short (e.g., 2 nucleotides) splint oligonucleotides, while achieving specificity of splint oligonucleotide hybridization and/or ligation, for instance, when splint oligonucleotide hybridization and ligation are performed in the same reaction mix and/or the same reaction condition. In some embodiments, using a low hybridization temperature, less denaturation, and/or more co-factors such as Mg²⁺ or other factors that promote hybridization can enable the use of shorter splint oligonucleotides.

In some embodiments, upon hybridization to the target nucleic acid molecule, the 5′ terminal nucleotide of the splint oligonucleotide is adjacent to the 3′ terminal nucleotide of the first probe or probe set, and the 3′ terminal nucleotide of the splint oligonucleotide is adjacent to the 5′ terminal nucleotide of the first probe or probe set. In some embodiments, the 5′ terminal nucleotide of the splint oligonucleotide and the 3′ terminal nucleotide of the first probe or probe set are separated by a nick or a gap of one or more nucleotides, e.g., 1, 2, 3, 4, 5, 6, 7, 8, 9, 10, or more nucleotides. In some embodiments, the 3′ terminal nucleotide of the splint oligonucleotide and the 5′ terminal nucleotide of the first probe or probe set are separated by a nick or a gap of one or more nucleotides, e.g., 1, 2, 3, 4, 5, 6, 7, 8, 9, 10, or more nucleotides. The nick can be ligated using any suitable ligase disclosed herein, and the gap can be filled using any suitable polymerase followed by ligation, for example, as described in Section III-A. In some embodiments, the first probe or probe set is circularized using a combination of gap-fill oligonucleotide hybridization and gap-fill polymerization (e.g., primer extension of the 3′ end of the first probe and primer extension of the 3′ end of a gap-fill oligonucleotide) to generate circularized gap-filled probe comprising a gap-filled region complementary to the gap sequence.

In some embodiments, the first probe or probe set is hybridized to the target nucleic acid, followed by contacting the biological sample with a library of splint oligonucleotides that compete for hybridization to the target nucleic acid (e.g., hybridization to the gap sequence in the target nucleic acid). In some embodiments, the hybridization of a splint oligonucleotide to the target nucleic acid and the ligation of the splint oligonucleotide to the circularizable probe are performed sequentially, e.g., the splint oligonucleotide hybridization is performed in a reaction condition or reaction mix, and the splint oligonucleotide ligation is performed in a different reaction condition or different reaction mix. In some embodiments, the hybridization of a splint oligonucleotide to the target nucleic acid and the ligation of the splint oligonucleotide to the first probe or probe set are performed in the same reaction condition or the same reaction mix. In some embodiments, any one or more of the splint oligonucleotides in the library are 2 nucleotides or more in length.

In some embodiments, the first probe or probe set and the library of splint oligonucleotides is contacted with the target nucleic acid at the same time, in the same reaction mix. In some embodiments, the first probe or probe set and the library of splint oligonucleotides is added to the target nucleic acid sequentially. For example, in some instances, the first probe or probe set and the library of splint oligonucleotides is premixed before contacting the biological sample with the mixture. In another example, two separate compositions comprising the first probe or probe set and the library of splint oligonucleotides, respectively, are contacted with the biological sample. In some embodiments, the hybridization of a splint oligonucleotide to the target nucleic acid and the ligation of the splint oligonucleotide to the first probe or probe set are performed in the same reaction condition or the same reaction mix. In some embodiments, any one or more of the splint oligonucleotides in the library are 2 nucleotides or more in length.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA. In some embodiments, the ligase is a recombinantly-produced ligase. In some examples, the recombinantly-produced ligase comprises a ligase isolated from Acanthocystis turfacea chlorella virus 1 (ATCV-1). In some embodiments, the ligase is fused to at least one polynucleotide-binding polypeptide (e.g., a HU protein, a functional variant, or a functional fragment thereof). An example of a suitable ligase is described in U.S. Patent Application Publication No. 2024/0150745, which is incorporated herein by reference in its entirety.

In some embodiments, the splint oligonucleotide comprises a sequence complementary to the gap sequence in the target nucleic acid molecule. In some embodiments, the biological sample is contacted with a library of splint oligonucleotides. In some embodiments, the library comprises at least about 2, at least about 4, at least about 10, at least about 20, at least about 50, at least about 100, or more oligonucleotides of different sequences. In some embodiments, the sequence diversity of the splint oligonucleotides in the library is such that at least or about 80%, at least or about 85%, at least or about 90%, at least or about 95%, or about 100% of the possible variant sequences in the gap sequence of the target nucleic acid in a sample have corresponding splint oligonucleotides in the library, e.g., the splint oligonucleotides comprise sequences that are complementary to the variant sequences in the target nucleic acid.

In some embodiments, the gap filling is performed under conditions permissive for specific hybridization of a splint oligonucleotide to its complementary sequence in the gap sequence in the target nucleic acid molecule, and/or specific hybridization of a first probe or probe set to the target nucleic acid molecule. In some embodiments, the first probe or probe set comprises hybridization regions that hybridize to the target nucleic acid molecule at sequences outside the gap sequence (e.g., at constant region sequences on the 5′ the 3′ of the hotspot for mutation), whereas the variant sequences in the gap sequence are complementary to the splint oligonucleotides (e.g., wildtype or mutant) in the library. In some embodiments, the circularized gap-filled probe is amplified by RCA (e.g., as described in Section IV), and the RCA product comprises multiple copies of the gap sequence in the target nucleic acid. In some embodiments, a sequence in the gap sequence in the RCA product is determined in situ, e.g., as described in Section V. In some embodiments, a sequence in the gap sequence in the RCA product is not sequenced using base-by-base sequencing, such as SBS, SBL, SBB, or avidity sequencing.

In some embodiments, the splint oligonucleotides is between about 6 and about 24 nucleotides in length. In some embodiments, any one or more of the splint oligonucleotides in the library is about 6, about 8, about 10, about 12, about 14, about 16, about 18, about 20, about 22, or about 24 nucleotides in length. Any two or more of the splint oligonucleotides in the library has the same length or different lengths. In some embodiments, the splint oligonucleotides in the library are of the same length.

In some embodiments, the variant sequence is at least 1, 2, 3, 4, 5, 6, 7, 8, 9, or more phosphodiester bonds from the 3′ or 5′ end of the gap sequence. In some embodiments, the sequence complementary to the variant sequence is at least 1, 2, 3, 4, 5, 6, 7, 8, 9, or more phosphodiester bonds from the 5′ or 3′ end of the splint oligonucleotide. In some embodiments, the variant sequence is at or near the central nucleotide(s) of the gap sequence. In some embodiments, the sequence complementary to the variant sequence is at or near the central nucleotide(s) of the splint oligonucleotide. In some embodiments, the sequence complementary to the variant sequence is no more than 1, no more than 2, no more than 3, no more than 4, no more than 5, or no more than 6 nucleotides from the central nucleotide(s) of the splint oligonucleotide.

In some embodiments, a method disclosed herein comprises hybridizing a gap-fill padlock probe on conserved regions that flank a gap sequence in a nucleic acid target sequence. For instance, the conserved regions are present in the majority or all RNA transcripts from the same gene (e.g., KRAS), and the gap sequences in the RNA transcripts may comprise one or more variant sequences (each of one or more bases), or regions to be interrogated (e.g., one or more SNPs) depending on the particular transcript. In some embodiments, the gap sequences comprise mutation hotspots. In some embodiments, a gap between the arms of a gap-fill padlock probe hybridized to conserved regions in the nucleic acid target is filled using the gap sequence as a template, thereby incorporating sequence information regarding the variant sequence(s) from the nucleic acid target into the gap-filled padlock probe. As used herein, in some instances, a gap sequence is an intervening sequence (of one or more bases) between a first target sequence and a second target sequence in a target nucleic acid, and the gap sequence is linked to the first and second target sequences via one or more phosphodiester bonds. In some embodiments, the first and second target sequences are targets of a first probe or probe set, such as arms of a gap-fill padlock probe that do not comprise a variant sequence or an interrogatory region. In some embodiments, the gap sequence comprises one or more variant sequences or regions to be interrogated, whereas the flanking first and second target sequences are constant or invariant among multiple target nucleic acid molecules. In some embodiments, the gap sequence comprises a nucleotide of interest. In some embodiments, the nucleotide of interest is a SNP. In some embodiments, the splint oligonucleotides compete with one another for hybridization to a gap sequence that contains particular variant sequence(s). For instance, in the case of KRAS, mutations occur most frequently in 5 bases in codons 12 and 13. In some embodiments, library of splint oligonucleotides is designed to cover all possible variants (or any subset thereof) in that region with a length of about 12 bases. In some embodiments, the splint oligonucleotides is between about 6 and about 18 bases. In some embodiments, with short splint oligonucleotides, a single mismatch of one base, especially when the mismatch is in the middle of a short splint oligonucleotide, can decrease the stability of the splint oligonucleotide hybridization and the fully correct matching splint oligonucleotide is favored. Compared to a one-piece circularizable probe strategy where one arm of the probe matches a conserved region in the target sequence and a mismatch on the other arm (e.g., the arm containing an SNP-interrogatory nucleotide) does not destabilize the arm matching the conserved region, the splint oligonucleotide approach allows competition among splint oligonucleotides and dissociation of mismatched splint oligonucleotides prior to probe ligation.

In some embodiments, the ligation of the splint oligonucleotides to the first probes or probe sets (e.g., gap-fill padlock probes) is performed using RNA-templated ligation. In some embodiments, the ligation is performed after hybridization of the splint oligonucleotide library and removing splint oligonucleotides mismatched on target nucleic acids, and the method comprises ligating splint oligonucleotides matched with target nucleic acids to the circularizable probes. In some embodiments, the ligation is performed simultaneously with splint oligonucleotide hybridization, e.g., a ligase is present during splint oligonucleotide hybridization. In some embodiments, the splint oligonucleotide library and a plurality of circularizable probes are contacted with the sample simultaneously or in any order. In some embodiments, the sample is contacted with the splint oligonucleotide library and the plurality of circularizable probes at the same time, and the splint oligonucleotide library and the plurality of circularizable probes are pre-mixed or not pre-mixed prior to contacting the sample. In some embodiments, the plurality of circularizable probes are hybridized to target nucleic acids in the sample before the splint oligonucleotide library is hybridized and ligated to the circularizable probes. In some embodiments, the splint oligonucleotide library is hybridized to target nucleic acids in the sample before the plurality of circularizable probes are hybridized and ligated to the splint oligonucleotides.

In some embodiments, one or more washes are performed between any of first probe or probe set hybridization, splint oligonucleotide hybridization, and ligation. In some embodiments, any one or more of the washes are stringent so that only completely complementary splint oligonucleotides remain bound to target nucleic acids after the wash(es). In some embodiments, any one or more of the washes are performed under less than stringent conditions. In some embodiments, any one or more of the washes are performed under extremely low stringency conditions, low stringency conditions, or medium stringency conditions.

In some embodiments, the first probe is circularized to generate a circularized gap-filled probe comprising a gap-filled region complementary to the gap sequence, wherein the gap-filled region is generated using gap-filling by polymerization, or gap-fill splint ligation, or a combination thereof. In some embodiments, after performing a gap-fill reaction on the first probe or probe set bound to the target RNA to generate a gap-filled probe or probe set, a ligation is performed to circularize the gap-filled probe or probe set. In some embodiments, the splint oligonucleotide is ligated to the first probe or probe set using the target RNA as a template and a ligase having an RNA-templated DNA or RNA ligase activity. In some instances, the ligase is a PBCV-1 DNA ligase or a T4 RNA ligase 2. In some embodiments, the extended probe (by polymerization) is ligated using the target RNA as a template and a ligase having an RNA-templated DNA or RNA ligase activity. In some cases, the extended probe is ligated to another end of the first probe or probe set to form a circularized gap-filled probe or probe set. For example, the ligase is a Chlorella virus DNA ligase (PBCV DNA ligase) or a T4 RNA ligase, optionally wherein the ligase is a PBCV-1 DNA ligase or a T4 RNA ligase 2. In some embodiments, the ligase is a recombinantly-produced ligase. In some examples, the recombinantly-produced ligase comprises a ligase isolated from Acanthocystis turfacea chlorella virus 1 (ATCV-1). In some embodiments, the ligase is fused to at least one polynucleotide-binding polypeptide (e.g., a HU protein, a functional variant, or a functional fragment thereof). An example of a suitable ligase is described in U.S. Patent Application Publication No. 2024/0150745, which is incorporated herein by reference in its entirety.

### IV. Rolling Circle Amplification (RCA)

Following formation of the circularized gap-filled first probe or probe set (e.g., as described in Section III), in some instances, a primer oligonucleotide is added for amplification. In some instances, the primer oligonucleotide is added with the first probe or probe set. In some instances, the primer oligonucleotide is added before or after the first probe or probe set is contacted with the sample. In some instances, the primer oligonucleotide for amplification of the circularized gap-filled probe comprises a sequence complementary to a target nucleic acid, as well as a sequence complementary to the first probe or probe set that hybridizes to the target nucleic acid. In some embodiments, a washing step is performed to remove any unbound probes, primers, etc. In some embodiments, the wash is a stringency wash. Washing steps can be performed at any point during the process to remove non-specifically bound probes, probes that have ligated, etc.

In some embodiments, a primer oligonucleotide for amplification of the circularized gap-filled probe comprises a single-stranded nucleic acid sequence having a 3′ end that is used as a substrate for a nucleic acid polymerase in a nucleic acid extension reaction. In some embodiments, the primer oligonucleotide comprises both RNA nucleotides and DNA nucleotides (e.g., in a random or designed pattern). In some embodiments, the primer oligonucleotide also comprises other natural or synthetic nucleotides described herein that can have additional functionality. In some embodiments, the primer oligonucleotide is about 6 bases to about 100 bases, such as about 25 bases.

In some embodiments, amplification of the circularized gap-filled probe is primed by the target RNA. The target RNA can optionally be immobilized in the biological sample. In some embodiments, the target RNA is cleaved by an enzyme (e.g., RNase H). In some embodiments, the target RNA is cleaved at a position downstream of the target sequences bound to the circularized gap-filled probe. In some aspects, the methods disclosed herein allow targeting of RNase H activity to a particular region in a target RNA that is adjacent to or overlapping with a target sequence for a probe or probe set. For example, a nucleic acid oligonucleotide is designed to hybridize to a complementary oligonucleotide hybridization region in the target RNA. In some embodiments, a nucleic acid oligonucleotide is used to provide a DNA-RNA duplex for RNase H cleavage of the target RNA in the DNA-RNA duplex. In some embodiments, the oligonucleotide binds to the target RNA at a position that overlaps with the target sequence of the probe or probe set by about 1 to about 20 nucleotides or by about 8 to about 10 nucleotides. The cleaved target RNA itself can then be used to prime RCA of the circularized gap-filled probe generated from a circularizable probe or probe set (e.g., target-primed RCA). In some cases, a plurality of nucleic acid oligonucleotides are used to perform target-primed RCA for a plurality of different target RNAs.

In any of the embodiments herein, the biological sample is contacted with the RNase H (and optionally with the nucleic acid oligonucleotide) before or during formation of the circularized gap-filled first probe or probe set. In some embodiments, the biological sample is contacted with the oligonucleotide and with the RNase H simultaneously or sequentially (in either order) before contacting the sample with the probe or probe set. In any of the embodiments herein, the biological sample are contacted with the RNase H (and optionally with the nucleic acid oligonucleotide) after formation of the circularized probe or probe set. In any of the embodiments herein, the RNase H comprises an RNase H1 and/or an RNAse H2. In some embodiments, RNase inactivating agents or inhibitors are added to the sample after cleaving the target RNA.

In some instances, upon addition of a DNA polymerase in the presence of appropriate dNTP precursors and other cofactors, the amplification primer is elongated by replication of multiple copies of the template. In some embodiments, the amplification step utilizes isothermal amplification or non-isothermal amplification. In some embodiments, after the formation of the hybridization complex and any subsequent circularization (such as ligation of, e.g., a first probe or probe set), the circularized gap-filled probe is rolling-circle amplified to generate a RCA product (e.g., amplicon) containing multiple copies of the sequence of the circularized gap-filled probe.

In some embodiments, RCPs are generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some embodiments, the polymerase is Phi29 DNA polymerase.

In some embodiments, the polymerase comprises a modified recombinant Phi29-type polymerase. In some embodiments, the polymerase comprises a modified recombinant Phi29, B103, GA-1, PZA, Phi15, BS32, M2Y, Nf, G1, Cp-1, PRD1, PZE, SF5, Cp-5, Cp-7, PR4, PR5, PR722, or L17 polymerase. In some embodiments, the polymerase comprises a modified recombinant DNA polymerase having at least one amino acid substitution or combination of substitutions as compared to a wildtype Phi29 polymerase. Exemplary polymerases are described in U.S. Pat. Nos. 8,257,954; 8,133,672; 8,343,746; 8,658,365; 8,921,086; and 9,279,155, all of which are herein incorporated by reference. In some embodiments, the polymerase is not directly or indirectly immobilized to a substrate, such as a bead or planar substrate (e.g., glass slide), prior to contacting a sample, although the sample may be immobilized on a substrate.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some aspects, during the amplification step, modified nucleotides are added to the reaction to incorporate the modified nucleotides in the amplification product (e.g., nanoball). In some embodiments, the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide reacts with an acrylic acid N-hydroxysuccinimide moiety. Examples of other amine-modified nucleotides comprise, but are not limited to, a 5-Aminoallyl-dUTP moiety modification, a 5-Propargylamino-dCTP moiety modification, a N⁶-6-Aminohexyl-dATP moiety modification, or a 7-Deaza-7-Propargylamino-dATP moiety modification. In some embodiments, the modified nucleotides comprises base modifications, such as azide and/or alkyne base modifications, dibenzylcyclooctyl (DBCO) modifications, vinyl modifications, trans-Cyclooctene (TCO), and so on.

In some embodiments, the primer extension reaction mixture can comprise a deoxynucleoside triphosphate (dNTP) or derivative, variant, or analogue thereof. In some aspects, the generated amplification product (e.g., RCA product) is DNA. In some instances, ribonucleotides are not incorporated into the amplification product. In some embodiments, the RCA product does not comprise a ribonucleotide.

In some embodiments, the primer extension reaction mixture can comprise a catalytic cofactor of the polymerase. In any of the preceding embodiments, the primer extension reaction mixture can comprise a catalytic di-cation, such as Mg²⁺ and/or Mn²⁺.

In some aspects, the amplification product (e.g., RCA product) are anchored to a polymer matrix. In some embodiments, the amplification products are immobilized within the matrix generally at the location of the nucleic acid being amplified, thereby creating a localized colony of amplicons. In some embodiments, the amplification products are immobilized within the matrix by steric factors. In some embodiments, the amplification products are also immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products (e.g., RCA products) are copolymerized and/or covalently attached to the surrounding matrix thereby preserving their spatial relationship and any information inherent thereto. In some embodiments, the RCA products are generated from DNA or RNA within a cell embedded in the matrix. In some embodiments, the RCA products can also be functionalized to form covalent attachment to the matrix preserving their spatial information within the cell thereby providing a subcellular localization distribution pattern. In some embodiments, the provided methods involve embedding RCA products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing or probe hybridization while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

### V. Detecting Variant Sequences in RCA Products

As discussed supra, e.g., in Sections I and III, detecting variant sequences in the form of DNA in RCA products offers several advantages, including higher ligase fidelity in DNA-templated ligation, as well as higher tolerance of mis-ligation due to amplification of the variant sequences in the form of RCA products from a gap-filled circular template that is more likely to faithfully incorporate the correct sequence information into the circularized gap-filled probe than RNA-templated ligation.

### A. Second Probes or Probe Sets

In some aspects, a second probe or probe set hybridizes to an amplification product of the circularized gap-filled probe generated from the first probe or probe set. In some aspects, detecting a ligation product of a second probe or probe set at a location in a biological sample comprises detecting the ligation product in situ.

In some embodiments, the second probe or probe set hybridizes to an amplification product of the circularized gap-filled probe generated from the first probe or probe set with the assistance of an Argonaute (Ago) protein. In some embodiments, the method comprises contacting the biological sample with the second probe or probe set and an Argonaute protein (e.g., in a pre-formed complex with the second probe or probe set). In some embodiments, the Argonaute protein is nuclease-active or nuclease deficient. Argonaute-mediated hybridization of a second probe or probe set to an amplification product of the circularized gap-filled probe may offer several advantages. For example, in some cases, Argonaute-mediated hybridization of a probe to a complementary sequence in the amplification product occurs more rapidly than probe hybridization in the absence of an Argonaute protein. In some embodiments, requirements for complementarity of the probe to the amplification product provides more stringent matching criteria than hybridization of free probes (e.g., not in a complex with an Argonaute protein), allowing for precise detection and discrimination of sequences in the amplification product that may share some sequence similarity.

Argonaute proteins can be nuclease-active (i.e., have slicer activity) or nuclease-deficient (i.e., lack slicer activity). In some embodiments, provided herein is a method comprising contacting a biological sample with a nuclease-deficient Argonaute protein in a complex with a second probe. In some embodiments, the second probe serves as a guide nucleic acid for the Argonaute protein. In some embodiments, the nuclease-deficient Argonaute protein comprises a detectable moiety such as a fluorescent label. In some embodiments, the second probe comprises a detectable moiety. In some embodiments, the method comprises detecting the bound Argonaute protein in a complex with a second probe at a location in the biological sample, thereby detecting the complementary sequence of the second probe at the location in the biological sample.

Any suitable Argonaute protein for binding a nucleic acid in a nucleic acid duplex without cutting can be used. Generally, Argonaute proteins contain 6 main domains (N-terminal, L1 (Linker 1), PAZ (Piwi-Argonaute-Zwille), L2 (Linker 2), MID (Middle) and PIWI (P-element induced wimpy testis) responsible for binding of a guide nucleic acid and recognition of a guide target sequence. In some embodiments, the Argonaute protein is an RNA-guided Argonaute, and the guide nucleic acid is an RNA molecule. In some embodiments, the Argonaute protein is a DNA-guided Argonaute, and the guide nucleic acid is a DNA molecule.

In some embodiments, the Argonaute protein is a naturally-occurring protein (e.g., naturally occurs in prokaryotic or eukaryotic cells). In some embodiments, the Argonaute protein is not a naturally-occurring protein (e.g., a variant or mutant protein). In some embodiments, the Argonaute protein is a recombinant protein. In some embodiments, the Argonaute protein is genetically engineered (such as an Argonaute protein described in WO 2019/222036, which is hereby incorporated by reference in its entirety). In some embodiments, the Argonaute protein is a slicer-dead Argonaute protein, meaning that it lacks cutting activity or is nuclease-dead. In some embodiments, the Argonaute protein has been modified (e.g., genetically engineered or mutated) to lack cutting activity. In some embodiments, lacking cutting activity means that the Argonaute protein is not capable of cutting a target nucleic acid. In some embodiments, lacking cutting activity means that the Argonaute protein does not cut the target nucleic acid. In some embodiments, an Argonaute protein that naturally lacks cutting activity or that has been modified to lack cutting activity is a slicer-dead Argonaute.

In some embodiments, the Argonaute protein is a eukaryotic Argonaute protein. Generally, eukaryotic Argonaute proteins can mediate binding of a target RNA with a guide nucleic acid of RNA. In some embodiments, an Argonaute protein is of plant, algal, fungal (e.g., yeast), or animal (e.g., human, rodent, fruit fly, cnidarian, echinoderm, nematode, fish, amphibian, reptile, bird, etc.) origin. In some embodiments, the Argonaute protein is a eukaryotic Argonaute protein that has been modified to lack cutting activity.

In some embodiments, the Argonaute protein is a slicer-dead Ago1, Ago2, Ago3, Ago4, PIWI 1, PIWIL 2, PIWI 3, or PIWI 4 (such as the Argonaute proteins described in WO 2007/048629, which is hereby incorporated by reference in its entirety). In some embodiments, the Argonaute protein is Ago2. In some embodiments, the Ago2 is Drosophila Ago2. In some embodiments, the Argonaute protein is a recombinant Drosophila Argonaute protein. In some embodiments, the Argonaute protein is expressed in a mammalian cell line. In some embodiments, the Argonaute protein is a Drosophila Argonaute protein expressed in a mammalian cell line. In some embodiments, a Drosophila Argonaute protein is expressed using a method such that a loading complex specific to Drosophila species is not provided to obtain guide-free proteins. In some embodiments, the Argonaute protein is a purified recombinant Drosophila Argonaute protein. In some embodiments, the Argonaute protein is expressed in an insect cell line, such as a Schneider 2 (S2) cell line. In some embodiments, the Argonaute protein is a Drosophila Argonaute protein expressed in an insect cell line, such as a S2 cell line. In some embodiments, the Drosophila Argonaute protein is loaded with the barcode-binding probe prior to contacting the biological sample.

In some embodiments, the slicer-dead Argonaute protein is a eukaryotic Argonaute protein from a mammalian organism. In some embodiments, the mammalian Argonaute protein is selected from mammalian AGO1, AGO2, AGO3, and AGO4. In some embodiments, the mammalian Argonaute protein is a human Argonaute protein. In some embodiments, the human Argonaute protein is a human AGO1 or AGO4 protein which naturally lacks slicer activity (See Faehnle et al. The making of a slicer: activation of a human Argonaute-1. Cell Reports 2015 Jun. 27, 3(6): 1901-1909, which is hereby incorporated by reference in its entirety). In some embodiments, the human Argonaute protein is a human AGO2 protein that has been modified to lack slicer activity (See McGeary et al., The Biochemical Basis of microRNA Targeting Efficacy. Science 2019 Dec. 20; 366(6472): eaav1741, which is hereby incorporated by reference in its entirety). In some embodiments, the human Argonaute protein is a human AGO3 protein that has been modified to lack slicer activity.

In some embodiments, a method provided herein comprises contacting the biological sample with a second probe or probe set that binds to the RCP. Examples of second probes or probe sets 330 can be seen in FIG. 3B. In some embodiments, the second probe or probe set 330 is a probe pair that comprises a first probe molecule 331 and the second probe molecule 332. In some embodiments, the second probe or probe set 330 comprises a constant region 333 for hybridizing to a sequence in the RCP 321. In some embodiments, the second probe or probe set 330 can be ligated at an optional second ligation 335. In some embodiments, the second probe or probe set 330 comprises an interrogatory region 334 for interrogating the variant sequence (indicated by X 322) in the gap sequence 323 in the RCP 321. The RCP 321 comprises a gap sequence 323 comprising one of multiple variant sequences (indicated by X 322) flanked by a first target sequence and second target sequence of the target nucleic acid molecule used as template for the gap fill reaction (e.g., as described in Section III). In some embodiments, the method further comprises ligating the first probe molecule 331 and the second probe molecule 332 of the second probe set 330 to generate a ligation product when the interrogatory region comprises a sequence complementary to the variant sequence in the RCP. In some embodiments, the method further comprises analyzing the ligation product to determine a location of the target RNA comprising the variant sequence in the biological sample. In some aspects, to interrogate the variant sequence or a complement thereof in the RCP, hybridization and ligation of the second probe or probe set is performed. In some aspects, hybridization and successful ligation of the second probe or probe set is indicative of the variant sequence or a complement thereof in the RCP.

In some embodiments, the second probe or probe set comprises a 5′ region and a 3′ region that hybridize to a target nucleotide of interest and an adjacent sequence to the target nucleotide of interest on an RCP. In some embodiments, upon hybridization of a second probe or probe set to a target RCP, the 3′ terminal nucleotide and the 5′ terminal nucleotide of the second probe or probe set are directly next to each other. In some instances, the 3′ terminal nucleotide and the 5′ terminal nucleotide of the second probe or probe set are directly ligated to each other. In some embodiments, upon hybridization of a second probe or probe set to a target RCP, the 3′ terminal nucleotide and the 5′ terminal nucleotide of the second probe or probe set are separated by a nick. In some embodiments, the second probe or probe set is not extended by polymerization or gap-filled prior to ligation of the second probe or probe set to generate a ligation product.

In some embodiments, a second probe or probe set disclosed herein comprises one or more barcode sequences. In some embodiments, a second probe or probe set comprises one or more barcode sequences. The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

In some embodiments, the barcode sequence are arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the number of distinct barcode sequences in a population of second probes or probe sets is less than the number of distinct targets of the second probes or probe sets, and yet the distinct targets may still be uniquely identified from one another, e.g., by encoding a second probe or probe set with a different combination of barcode sequences. However, not all possible combinations of a given set of barcode sequences need be used. For instance, each second nucleic acid probe or probe set may contain 1, 2, 3, 4, 5, 6, 7, 8, 9, 10, 11, 12, 13, 14, 15, etc. or more barcode sequences. In some embodiments, a population of second nucleic acid probes or probe sets each contain the same number of barcode sequences, although in other cases, there may be different numbers of barcode sequences present on the various second probes or probe sets. In some embodiments, the barcode sequences or any subset thereof in the population of second nucleic acid probes or probe sets are independently and/or combinatorially detected and/or decoded.

In some embodiments, the second probe or probe set comprises a barcode region comprising one or more barcode sequences associated with the target RNA or a sequence thereof, for example, as shown in FIG. 4 or FIG. 5. In some embodiments, the barcode region is not complementary to the target RNA or sequence thereof. In some embodiments, the second probe or probe set comprises an asymmetric split probe pair, where the barcode region is located on the shorter probe of the probe pair and the probes are connected to form a circular probe. In some embodiments, the target binding region (e.g., the RCP binding region) of one probe is shorter than that of the other probe. In some embodiments, each probe comprises a splint binding region complementary to a portion of a splint oligonucleotide (which is separate from the target, e.g., the RCP) such that the probes are configured to be ligated using the splint oligonucleotide as a template, and the splint binding region of one probe is shorter than that of the other probe. In some embodiments, the second probe or probe set comprises an asymmetric split probe pair, where the barcode region is located on the shorter probe of the probe pair and the probes are connected to form a linear probe that is not circular. In some embodiments, the target binding region (e.g., the RCP binding region) of one probe is shorter than that of the other probe. In some embodiments, the barcode region is located in an overhang of the shorter probe hybridized to the RCP. In some embodiments, the barcode region is located in a 5′ overhang of the shorter probe. In some embodiments, the barcode region is located in a 3′ overhang of the shorter probe. In some embodiments, the longer probe comprises a 3′ overhang upon hybridization to the RCP. In some embodiments, the longer probe comprises a 5′ overhang upon hybridization to the RCP. In some embodiments, the barcode region in the shorter probe and/or the barcode region in the longer probe is detected using detectably labeled probes. In some embodiments, the barcode region in the shorter probe and/or the barcode region in the longer probe is detected using sequential rounds of binding of intermediate probes, wherein the intermediate probes are detected via binding to detectably labeled probes. In some embodiments, compared to probe pairs with probes having equal length (e.g., when the probes have target binding regions of equal length), a split probe pair comprising a shorter probe with the barcode region allows the shorter probe to be washed off more easily if the probes are not ligated to each other to form a circular probe or a linear probe that is not circular. In some embodiments, the barcode-bearing probe (the shorter probe) being more easily washed off provides an advantage for reducing background signal.

In some embodiments, the second probe or probe set 330 comprises the interrogatory region 334 and a constant region 333 complementary to the RCP, as shown in FIG. 3B. In some embodiments, the RCP comprises the same sequence as the target RNA, including the gap sequence and flanking probe hybridization regions. In some embodiments, the interrogatory region comprises a sequence complementary to a portion of the gap sequence. In some embodiments, the second probe or probe set comprises a single molecule comprising the interrogatory region and the constant region, each at one end of the probe molecule. In some embodiments, the second probe or probe set comprises two molecules, wherein a first molecule of the second probe set comprises the interrogatory region, and a second molecule of the second probe set comprises the constant region. In some embodiments, the interrogatory region is shorter in length compared to the constant region. In some embodiments, the constant region comprises a sequence complementary to a portion of the gap sequence. In some embodiments, the interrogatory region comprises a 3′ sequence of the second probe or probe set complementary to a 3′ portion of the gap sequence, whereas the constant region comprises a 5′ sequence of the second probe or probe set complementary to a 5′ portion of the gap sequence. In some embodiments, the interrogatory region comprises a sequence complementary to a portion of the gap sequence and a sequence complementary to a portion of the first or second target sequence. In some embodiments, the constant region comprises a sequence complementary to a portion of the gap sequence and a sequence complementary to a portion of the second or first target sequence.

In some embodiments, the constant region is common among a plurality of second probes or probe sets each comprising a different interrogatory region for a different variant sequence of the target RNA. In some embodiments, the interrogatory region is 3′ or 5′ to the constant region in the second probe or probe set. In some embodiments, the interrogatory region and the constant region in the second probe or probe set are equal in length. In other embodiments, the interrogatory region is shorter or longer than the constant region. In some embodiments, the interrogatory region and/or the constant region in the second probe or probe set is between about 5 and about 50 nucleotides in length. In some embodiments, the interrogatory region and/or the constant region in the second probe or probe set is between about 15 and about 25 nucleotides in length. In some embodiments, the interrogatory region and/or the constant region is optionally about 20 nucleotides in length. In some embodiments, the interrogatory region and/or the constant region in the second probe or probe set comprises a 5′ flap configured to be cleaved by a structure-specific endonuclease.

In some embodiments, the second probe or probe set is DNA. In some embodiments, the second probe or probe set is ligated using a ligase having a DNA-templated DNA ligase activity. In some embodiments, the second probe or probe set is ligated using a ligase selected from the group consisting of a Thermus thermophilus (Tth) DNA ligase, a Thermus aquaticus (Taq) DNA ligase, a T3 DNA ligase, a T4 DNA ligase, a T7 DNA ligase, an E. coli DNA ligase, a 9°N DNA ligase, a Chlorella virus DNA ligase (PBCV DNA ligase), and a T4 RNA ligase 2.

In some embodiments, the variant sequence comprises a single nucleotide of interest, the interrogatory region in the second probe or probe set comprises a nucleic acid residue complementary to the single nucleotide of interest, and the nucleic acid residue is no more than 1, 2, 3, 4, or 5 phosphodiester bonds from a 3′ or 5′ end of the second probe or probe set. In some embodiments, the interrogatory region comprises a 3′ terminal nucleic acid residue that is complementary to the single nucleotide of interest. In some embodiments, the second probe or probe set comprises a 3′ terminal nucleic acid residue complementary to the nucleotide immediately 3′ (connected by a phosphodiester bond) to the single nucleotide of interest, and after cleaving the 5′ flap, the second probe or probe set comprises a 5′ terminal nucleic acid residue complementary to the single nucleotide of interest. In alternate embodiments, the interrogatory region comprises a 5′ terminal nucleic acid residue that is complementary to the single nucleotide of interest. In some embodiments, the second probe or probe set comprises a 5′ flap configured to be cleaved by a structure-specific endonuclease to release the 5′ flap from the second probe or probe set and allow ligation of the second probe or probe set. In alternate embodiments, the second probe or probe set comprises a 3′ terminal nucleic acid residue complementary to the single nucleotide of interest, and the released 5′ flap comprises a 3′ terminal nucleic acid residue complementary to the single nucleotide of interest.

In some embodiments, a ligation product of a second probe or probe set hybridized to an RCP is generated using the RCP as a template for the ligation. In some embodiments, ligation of the second probe or probe set hybridized to an RCP is generated using the RCP. In some examples, the ligation is performed using any suitable ligase, e.g., any ligases described herein. In some embodiments, multiple molecules of a ligation product comprising the same sequence are generated using DNA-templated ligation, each using a copy of a unit sequence comprising a variant nucleotide or sequence in the RCP as a template. In some embodiments, multiple ligation products comprising different sequences are generated using DNA-templated ligation, each using a different variant nucleotide or sequence as a template. In some embodiments, a plurality of detectably labeled probes each configured to directly or indirectly bind to one or more ligation products are used to detect the ligation products, and the ligation products can be of the same sequence or of different sequences each corresponding to a different variant nucleotide or sequence of interest.

In some embodiments, the generated ligation product of the second probe or probe set hybridized to the RCP is not amplified. In some embodiments, the generated ligation product of the second probe or probe set hybridized to the RCP is not circular. In some embodiments, the generated ligation product of the second probe or probe set hybridized to the RCP is linear. In some embodiments, the generated ligation product of the second probe or probe set hybridized to the RCP is not used as template to perform an additional amplification reaction (e.g., RCA). In some embodiments, the ligation product of the second probe or probe set hybridized to the RCP is detected.

### B. In Situ Detection of Ligation Products

In some aspects, the provided methods involve analyzing, e.g., detecting or determining, one or more nucleic acid sequences such as gap sequences in target nucleic acids. In some aspects, the provided methods comprise detecting or determining one or more nucleic acid sequences associated with gap sequences in target nucleic acids. In some aspects, the provided methods comprise detecting or determining one or more nucleic acid sequences corresponding to gap sequences in target nucleic acids. In some cases, the analysis is performed on one or more images captured, and may comprise processing the image(s) and/or quantifying signals observed. In some embodiments, the analysis comprises detecting a sequence (e.g., a gap sequence) present in the sample. In some embodiments, the analysis comprises quantification of puncta (e.g., if amplification products are detected). In some embodiments, the obtained information may be compared to a positive and negative control, or to a threshold of a feature to determine if the sample exhibits a certain feature or phenotype. In some cases, the information comprises signals from a cell, a region, and/or comprise readouts from multiple detectable labels. In some cases, the analysis further comprises displaying the information from the analysis or detection step. In some embodiments, software is used to automate the processing, analysis, and/or display of data.

In some embodiments, the detection or determination comprises hybridizing one or more probes to nucleic acid molecules such as RCPs (e.g., described in Section IV) and/or to the second probes or probe sets (e.g., as described in Section V.A). In some embodiments, the ligation product generated from ligating the second probe or probe set is detected. In some embodiments, the in situ detection herein comprises sequential hybridization of probes, e.g., sequencing by hybridization and/or sequential in situ fluorescence hybridization. In some embodiments, sequential fluorescence hybridization comprises sequential hybridization of the detectably labeled probes disclosed herein. In some embodiments, a method disclosed herein comprises sequential hybridization of detectably labeled probes and intermediate probes that are not detectably labeled per se but are capable of binding (e.g., via nucleic acid hybridization) and being detected by detectably labeled probes, such as fluorescently labeled probes. Exemplary methods comprising sequential fluorescence hybridization of probes are described in US 2019/0161796, US 2020/0224244, US 2022/0010358, US 2021/0340618, WO 2020/123742, and WO 2021/138676, all of which are incorporated herein by reference.

In some embodiments, a detectably labeled probe hybridizes to a detectable region in a ligation product of a second probe or probe set disclosed herein. In some embodiments, the ligation product of the second probe or probe set is circular and the circular ligation product may but does not need to be amplified using RCA for detection. In some embodiments, the detectable region is in the circular ligation product, such as a barcode region in the circular ligation product shown in FIG. 4 or FIG. 5. In some embodiments, the ligation product of the second probe or probe set is linear and not circular. In some embodiments, the detectable region is in a 5′ overhang and/or a 3′ overhang of the ligation product. In some embodiments, the detectable region is a split region, e.g., a portion of the detectable region is in the 5′ overhang and another portion of the detectable region is in the 3′ overhang of the ligation product. In some embodiments, the detectable region is in the 5′ overhang of the ligation product. In some embodiments, the detectable region is in the 3′ overhang of the ligation product. In some embodiments, a first portion of the detectable region is in the 3′ overhang and a second portion of the detectable region is in the 5′ overhang of the ligation product.

In some embodiments, the second probe set comprises a pair of probe molecules that hybridize to the RCP and one of the probe molecules of the probe pair comprises an interrogatory nucleotide for the SNV (e.g., indicated by X′ in FIG. 6A). In some instances, the pair of probes is ligated to form a ligation product that is detectably labeled (e.g., as shown in FIG. 6A). In some embodiments, the second probe set comprises a pair of probe molecules which are configured to hybridize to the RCP and one of the probe molecules of the probe pair comprises an interrogatory nucleotide for the SNV (e.g., indicated by X′ in FIG. 6B). In some instances, the pair of probes is ligated to form a ligation product that comprises a detectable region configured to hybridize to a detectably labeled probe (e.g., as shown in FIG. 6B). In some embodiments, detection of the ligated second probe set is performed prior to detection of barcode regions in the RCP as shown in FIG. 5. In some embodiments, detection of the ligated second probe set is performed at the same time as detection of barcode regions in the RCP as shown in FIG. 5. In some embodiments, detection of the ligated second probe set is performed after detection of barcode regions in the RCP as shown in FIG. 5.

Provided herein are methods involving the use of one or more probes for analyzing one or more target nucleic acid(s), such as variant sequences in one or more target nucleic acids present in a cell or a biological sample, such as a tissue sample. In some embodiments, the probes include a plurality of second probes or probe sets and a plurality of detectably labeled probes for combinatorially decoding the barcode regions in the second probes or probe sets that are specifically hybridized and ligated using the variant sequences in the RCPs. In some embodiments, the probes include a plurality of detectably labeled probes for combinatorially decoding the barcode regions in the RCPs. Using sequential probe hybridization, the provided embodiments, in some instances, are employed for in situ detection of variant sequences in target nucleic acids in a cell, e.g., in cells of a biological sample or a sample derived from a biological sample, such as a tissue section on a solid support, such as on a transparent slide.

In some aspects, provided herein are in situ assays using microscopy as a readout, e.g., hybridization, or other detection or determination methods involving an optical readout. In some aspects, detection or determination of a sequence of one, two, three, four, five, or more nucleotides of a gap sequence in a target nucleic acid is performed in situ in a cell and/or in an intact tissue. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product or hybridization complex). In some embodiments, a provided method is quantitative and preserves the spatial information within a tissue sample without physically isolating cells or using homogenates. In some embodiments, the present disclosure provides methods for high-throughput profiling of target nucleic acids in situ in a large number of cells, tissues, organs or organisms.

In some aspects, the provided methods comprise imaging the amplification product (e.g., RCA product) of a first probe or probe set (e.g., as described in Section III) and the ligation product of a second probe or probe set (e.g., as described in Section V) via binding of detectably labeled probes (e.g., detection oligonucleotides each comprising a fluorescent label), and detecting the detectable labels, for instance, in sequential probe hybridization and detection cycles.

As shown in FIG. 4, a method disclosed herein comprises contacting the biological sample with a second probe or probe set that binds to the RCP. In some embodiments, the second probe or probe set comprises an interrogatory region for interrogating the variant sequence in the RCP, and a barcode region. In some embodiments, the barcode region in the second probe or probe set is associated with the variant sequence in the target RNA. In some embodiments, the method further comprises ligating the second probe or probe set to generate a ligation product when the interrogatory region comprises a sequence complementary to the variant sequence in the RCP. In some embodiments, only the matching second probe or probe set (e.g., comprising X′ in FIGS. 4-6) is ligated and detected using hybridization of detectably labeled probes. In some embodiments, the method further comprises removing molecules of probes or probe sets that are not ligated due to a mismatch with the variant sequence in the RCP (for example, performing one or more wash steps). In some embodiments, the method further comprises contacting the biological sample with detectably labeled probes in sequential cycles. In some embodiments, the detectably labeled probes hybridize directly to sequence(s) in the ligated second probes or probe sets. In some embodiments, in each cycle, a detectably labeled probe is directly or indirectly bound to a barcode sequence in the barcode region and a signal or absence thereof associated with the detectably labeled probe is recorded at a location in the biological sample, thereby generating a signal code sequence corresponding to the barcode region. In some embodiments, the signal code sequence comprises the signal or absence thereof recorded at the location in each of the sequential cycles. In some embodiments, the method further comprises using the signal code sequence to identify the variant sequence of the target RNA at the location in the biological sample.

In some embodiments, the biological sample is contacted with a plurality of second probes or probe sets each comprising a different interrogatory region for a different variant sequence of the target RNA and a different barcode region corresponding to the different variant sequence. In some embodiments, in each cycle, the detectably labeled probe is hybridized to an intermediate probe which in turn hybridizes to the barcode region. In some embodiments, in two or more of the sequential cycles, the intermediate probes are hybridized to the same barcode sequence or different barcode sequences in the barcode region. In some embodiments, in each of the sequential cycles, the intermediate probe is hybridized to the same barcode sequence or a different barcode sequence in the barcode region. In some embodiments, in each cycle, the detectably labeled probe is hybridized to the barcode region. In some embodiments, in two or more of the sequential cycles, the detectably labeled probes are hybridized to different barcode sequences in the barcode region. In some embodiments, in each of the sequential cycles, the detectably labeled probe is hybridized to a different barcode sequence in the barcode region. In some embodiments, the barcode region comprises two or more non-overlapping barcode sequences. In some embodiments, the barcode region comprises two or more overlapping barcode sequences. In some embodiments, each pair of adjacent barcode sequences in the barcode region are partially overlapping. In some embodiments, the ligation product of the second probe or probe set is circular. In some embodiments, the ligation product of the second probe or probe set is not circular.

As shown in FIG. 5, a method disclosed herein comprises contacting the biological sample with a first probe or probe set. In some embodiments, the first probe or probe set comprises a first probe region and a second probe region that bind to a first target sequence and a second target sequence, respectively, in a target RNA in the biological sample, and a complement of a first barcode region. In some embodiments, the first barcode region comprises one or more barcode sequences associated with the target RNA or a sequence thereof but not associated with the variant sequence. In some embodiments, the first barcode region comprises two or more non-overlapping barcode sequences. In some embodiments, the first barcode region comprises two or more overlapping barcode sequences. In some embodiments, each pair of adjacent barcode sequences in the first barcode region are partially overlapping. In some embodiments, the first and second target sequences flank a gap sequence in the target RNA, and wherein the gap sequence comprises a variant sequence. In some embodiments, the method further comprises performing a gap-fill reaction on the first probe or probe set to generate a gap-filled probe or probe set. In some embodiments, the method further comprises circularizing the gap-filled probe or probe set to generate a circularized gap-filled probe comprising a gap-filled region complementary to the gap sequence. In some embodiments, the method further comprises using a polymerase to amplify the circularized gap-filled probe to generate a rolling circle amplification product (RCP) in the biological sample.

In some embodiments, the method further comprises contacting the biological sample with a second probe or probe set that binds to the RCP. In some embodiments, the second probe or probe set comprises an interrogatory region for interrogating the variant sequence in the RCP, and a second barcode region. In some embodiments, the second barcode region consists of one barcode sequence corresponding to a base selected from the group consisting of A, T, C, and G. In some embodiments, the barcode sequence corresponding to the base is common among a plurality of second probes or probe sets targeting different target RNAs or different sequences of the same target RNA. In some embodiments, the second barcode region is associated with the variant sequence in the target RNA. In some embodiments, the method further comprises ligating the second probe or probe set to generate a ligation product when the interrogatory region comprises a sequence complementary to the variant sequence in the RCP. In some embodiments, the ligation product of the second probe or probe set is circular and is optionally amplified using rolling circle amplification for detection. In some embodiments, the ligation product of the second probe or probe set is not circular. In some embodiments, a single cycle of hybridizing a detectably labeled probe to a sequence in the ligated second probes or probe set is performed to identify the variant sequence (e.g., SNP).

In some aspects, one or more nucleic acid sequences of the generated RCP (using the gap-filled probe or probe set as template) is detected. In some aspects, one or more nucleic acid sequences of the generated ligation product (using the second probe or probe set) is detected. In some aspects, one or more nucleic acid sequences of the generated RCP (using the gap-filled probe or probe set as template) and one or more nucleic acid sequences of the generated ligation product (using the second probe or probe set) is detected. In some examples, the detection is performed by hybridizing a detectably labeled probe to the one or more nucleic acid sequences. In some instances, one or more nucleic acid sequences of the generated ligation product (using the second probe or probe set) is detected separately from the one or more nucleic acid sequences of the generated RCP (using the gap-filled probe or probe set as template). In some instances, one or more nucleic acid sequences of the generated ligation product (using the second probe or probe set) is detected at the same time as the one or more nucleic acid sequences of the generated RCP (using the gap-filled probe or probe set as template).

In some embodiments, the method further comprises removing molecules of probes or probe sets that are not ligated due to a mismatch with the variant sequence in the RCP. In some embodiments, the method further comprises contacting the biological sample with detectably labeled probes in sequential cycles. In some embodiments, in each cycle, a detectably labeled probe is directly or indirectly bound to a barcode sequence in the first or second barcode region and a signal or absence thereof associated with the detectably labeled probe is recorded at a location in the biological sample, thereby generating a signal code sequence corresponding to the first and second barcode regions. In some embodiments, in each cycle, the detectably labeled probe is hybridized to the first barcode region or the second barcode region. In some embodiments, in two or more of the sequential cycles, the detectably labeled probes are hybridized to different barcode sequences in the first barcode region or different barcode sequences in the second barcode region. In some embodiments, in each of the sequential cycles for decoding the first barcode region, the detectably labeled probe is hybridized to a different barcode sequence in the first barcode region. In some embodiments, each pair of adjacent barcode sequences in the first barcode region are partially overlapping. In some embodiments, in each cycle, the detectably labeled probe is hybridized to an intermediate probe which in turn hybridizes to the first barcode region or the second barcode region. In some embodiments, in two or more of the sequential cycles, the intermediate probes are hybridized to the same barcode sequence or different barcode sequences in the first barcode region or the second barcode region. In some embodiments, in each of the sequential cycles for decoding the first barcode region, the intermediate probe is hybridized to the same barcode sequence or a different barcode sequence in the first barcode region.

In some embodiments, the signal code sequence comprises the signal or absence thereof recorded at the location in each of the sequential cycles. In some embodiments, the method provided herein further comprises using the signal code sequence to identify the variant sequence of the target RNA at the location in the biological sample.

In some embodiments, all or a portion of the RCPs (e.g., described in Section IV) and/or the second probes or probe sets (e.g., as described in Section V) is detected using a base-by-base sequencing method, e.g., SBS or SBB. In some embodiments, base-by-base sequencing is performed directly on a sequence in the ligated second probes or probe sets. In some embodiments, the base-by-base sequencing is performed in situ at the location of the target RNA in the cell or tissue sample. In some embodiments, the base-by-base sequence is performed to detect the nucleotide of interest (e.g., to determine identity of the SNP). In some embodiments, the base-by-base sequence is performed to detect a barcode sequence (e.g., in a second probe or probe set) associated with the nucleotide of interest (e.g., the SNP). In some embodiments, the biological sample is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

In some embodiments, the base-by-base sequencing comprises using a polymerase that is fluorescently labeled. In some embodiments, the base-by-base sequencing comprises using a polymerase-nucleotide conjugate comprising a fluorescently labeled polymerase linked to a nucleotide moiety that is not fluorescently labeled. In some embodiments, the base-by-base sequencing comprises using a multivalent polymer-nucleotide conjugate comprising a polymer core, multiple nucleotide moieties, and one or more fluorescent labels.

In some embodiments, sequencing is performed by sequencing-by-synthesis (SBS). In some embodiments, a sequencing primer is complementary to sequences at or near the one or more barcode(s). In such embodiments, sequencing-by-synthesis comprises reverse transcription and/or amplification in order to generate a template sequence from which a primer sequence can bind. Example SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing is performed by sequencing-by-binding (SBB). Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e. different types of ternary complexes differing in the type of nucleotide it contains) or by separately delivering the reagents needed to form each type of ternary complex. In some instances, the labeling comprises fluorescence labeling of, e.g., the cognate nucleotide or the polymerase that participate in the ternary complex.

In some embodiments, sequencing is performed by sequencing-by-avidity (SBA). Some aspects of SBA approaches are described in U.S. Pat. No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated.

In some embodiments, a signal associated with a detectably labeled oligonucleotide is measured and quantitated. In some embodiments, the terms “label” and “detectable label” comprise a directly or indirectly detectable moiety that is associated with (e.g., conjugated to) a molecule to be detected, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

In some embodiments, the term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

In some embodiments, an RCP or a probe (e.g., second probes or probe sets) disclosed herein comprises one or more detectably labeled, e.g., fluorescent, nucleotides. In some embodiments, the one or more detectably labelled nucleotides are incorporated during generation of the RCP (e.g., during RCA) or the probe. Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). For exemplary methods for custom synthesis of nucleotides having other fluorophores, see, Henegariu et al. (2000) Nature Biotechnol. 18:345.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles are used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a polynucleotide sequence is indirectly labeled, especially with a hapten that is then bound by a capture agent, e.g., as disclosed in U.S. Pat. Nos. 5,344,757, 5,702,888, 5,354,657, 5,198,537 and 4,849,336, and PCT publication WO 91/17160. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of an RCP and/or a ligation product disclosed herein. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of an RCP and/or a ligation product disclosed herein. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECS™), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PS™, photon scanning tunneling microscopy (PS™), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXS™), and intact tissue expansion microscopy (exM).

### VI. Spatial Assays

In some embodiments, the detecting of a ligation product of a second probe or probe set at a location in a biological sample comprises detecting a location on a spatial array of capture probes, in which spatial information from a biological sample is preserved. In some embodiments, after hybridization and ligation of a second probe or probe set to an RCP generated in a sample, the assay may further comprise one or more steps for transferring the ligation product (or a product or derivative thereof) to an array for spatial assay (e.g., performing NGS sequencing to determine one or more sequences of the oligonucleotides captured on the array). In some embodiments, a spatial assay disclosed herein is performed without the in situ assays. In some embodiments, the spatial assay disclosed herein is performed following an in situ assay (e.g., using fluorescence microscopy as a readout). In some embodiments, RCA is performed as described in Section V as an enrichment method to increase the number of ligation products to be generated using the second probe or probe set and thus increase the number of reads to be detected.

In one aspect, provided herein are methods, compositions, apparatus, and systems for spatial analysis of a biological sample, for example, a spatial array-based analysis. Non-limiting aspects of spatial analysis methodologies are described in U.S. Pat. Pub. No. 10,308,982; U.S. Pat. Pub. No. 9,879,313; U.S. Pat. Pub. No. 9,868,979; Liu et al., bioRxiv 788992, 2020; U.S. Pat. Pub. No. 10,774,372; U.S. Pat. Pub. No. 10,774,374; WO 2018/091676; U.S. Pat. Pub. No. 10,030,261; U.S. Pat. Pub. No. 9,593,365; U.S. Pat. Nos. 10,002,316; 9,727,810; U.S. Pat. Pub. No. 10,640,816; Rodriques et al., Science 363(6434):1463-1467, 2019; U.S. Pat. No. 11,447,807; Lee et al., Nat. Protoc. 10(3):442-458, 2015; U.S. Pat. Pub. No. 10,179,932; U.S. Pat. No. 11,085,072; U.S. Pat. Pub. No. 10,138,509; Trejo et al., PLoS ONE 14(2):e0212031, 2019; U.S. Patent Application Publication No. 2018/0245142; Chen et al., Science 348(6233):aaa6090, 2015; Gao et al., BMC Biol. 15:50, 2017; WO 2017/144338; US 2018/0372736; US 2022/0290228; U.S. Pat. No. 11,597,965; WO 2011/094669; U.S. Pat. Nos. 7,709,198; 8,604,182; 8,951,726; 9,783,841; 10,041,949; WO 2016/057552; US 2021/0238665; U.S. Pat. Pub. No. 10,370,698; U.S. Pat. No. 10,724,078; U.S. Pat. Pub. No. 10,364,457; U.S. Pat. Pub. No. 10,317,321; US 2021/0395796; US 2020/0239946; U.S. Pat. Nos. 10,059,990; 11,505,819; 11,104,936; and Gupta et al., Nature Biotechnol. 36:1197-1202, 2018, all of which are herein incorporated by reference in their entireties, and can be used herein in any combination. Further non-limiting aspects of spatial analysis methodologies are described herein.

In some embodiments, the spatial assays disclosed herein comprise capturing a targeted analyte (e.g., a ligation product of a second probe or probe set). In some aspects, the biological sample is on the substrate (e.g., a cover slip with sufficient strength comprising the capture array). In some embodiments, the biological sample is on a second substrate. The biological sample may be positioned between the first substrate (e.g., substrate comprising the capture array) and the second substrate (e.g., slide comprising the biological sample) such that the capture agents are allowed to capture the ligation products or derivatives thereof. In some embodiments, the biological sample is processed to release the ligation products or portion thereof. The permeabilization step (e.g., using Proteinase K) allows the ligation products to migrate onto the capture array substrate and be captured by the capture probes. In some aspects, the permeabilization may be combined with lysing.

In some embodiments, a method disclosed herein comprises transferring one or more analytes (e.g., the ligation product of the second probe or probe set) from a biological sample to an array of features on a substrate, each of which is associated with a unique spatial location on the array. Each feature may comprise a plurality of capture agents (e.g., capture probes) capable of capturing one or more nucleic acid molecules, and each of the capture agents of the same feature may comprise a spatial barcode corresponding to a unique spatial location of the feature on the array. In some embodiments, the method comprises capturing the ligation product or a portion thereof by a capture agent (e.g., capture probe). The capture probe comprises a capture domain that binds to a capture sequence in the ligation product or a complement thereof. One or more reactions (e.g., extension, and/or ligation) are performed to generate a spatially labeled polynucleotide sequence comprising (i) a sequence of the captured ligation product or a complement thereof, and (ii) a sequence of the spatial barcode (e.g., spatial barcode of the capture probe) or complement thereof. Subsequent analysis of the transferred analytes includes determining the identity of the analytes and the spatial location of each analyte within the sample. The spatially labeled polynucleotide or a portion thereof may be removed from the substrate (e.g., capture array) for sequencing using any suitable nucleic acid sequencing techniques, including next-generation sequencing (NGS). In some embodiments, the sequence of the spatially labeled polynucleotide is determined to detect the spatial barcode and the ligation product. All or part of the sequence of the generated spatially labeled polynucleotide may be determined. The spatial location of each analyte (e.g., ligation product or complement thereof) within the sample is determined based on the feature to which each analyte is bound in the array, and the feature's relative spatial location within the array.

In some embodiments, a method disclosed herein comprises associating a spatial barcode with one or more analytes (e.g., ligation products disclosed herein), in one or more cells such as neighboring cells, such that the spatial barcode identifies the one or more analytes, and/or contents of the one or more cells, as associated with a particular spatial location.

In some embodiments, a method disclosed herein comprises driving target analytes out of a cell and towards a spatially-barcoded array. FIG. 7 depicts an embodiment where the biological sample is contacted with a spatially-barcoded array populated with capture probes. The sample can be permeabilized, allowing the ligation products of the second probes or probe sets (or molecules comprising complementary sequences of the ligation products) to migrate away from the sample and toward the array. The ligation product or a molecule comprising a complementary sequence of the ligation product can interact with a capture probe on the spatially-barcoded array. Once the ligation product or a complement thereof is captured (e.g., via hybridization or splint ligation to the capture probe), the sample is optionally removed from the array and the captured molecules are processed (e.g., using primer extension and/or ligation to incorporation sequence information of the target nucleic acid including the variant sequence and the spatial barcode sequence information) and analyzed in order to obtain spatially-resolved analyte information.

In some embodiments, a method disclosed herein comprises delivering or driving spatially-barcoded nucleic acid molecules (e.g., capture probes) towards and/or into or onto a sample. In some embodiments, a method disclosed herein comprises cleaving spatially-barcoded nucleic acid molecules (e.g., capture probes) from an array and driving the cleaved nucleic acid molecules towards and/or into or onto a sample. Alternatively, the sample may be permeabilized and fixed/crosslinked to restrict mobility of one or more target analytes, while allowing spatially-barcoded capture probes to migrate towards and/or into or onto the sample. Once the spatially-barcoded capture probe is associated with a particular analyte (e.g., a ligation product), the sample can be optionally removed for analysis. The sample can be optionally dissociated before analysis. Once the tagged analyte or cell is associated with the spatially-barcoded capture probe, the capture probes can be analyzed to obtain spatially-resolved information about the tagged analyte or cell.

Also disclosed herein are methods for an integrated in situ spatial assay comprising analyzing a first target analyte (e.g., a first variant sequence of a first target nucleic acid) using in situ analysis (e.g., using fluorescent microscopy as a read out, for instance as shown in FIG. 4 or FIG. 5) and analyzing a second target analyte (e.g., a second variant sequence (different from the first variant sequence) of the first target nucleic acid, a second target nucleic acid different from the first target nucleic acid, a variant sequence of the second target nucleic acid) using an array of capture probes (e.g., analyzing transcripts captured on the array using NGS sequencing of spatially barcoded nucleic acid molecules, or analyzing ligation products, for instance as shown in FIG. 7). The in situ analysis of the first target analyte may be performed either before, concurrently with, or after analyzing the second target analyte with the array of capture probes. In some embodiments, the second target nucleic acid is targeted by one or more nucleic acid probes complementary to the second target nucleic acid (e.g., a cDNA molecule generated from an mRNA molecule). After in situ detection of the first target analyte, the sample may be decrosslinked to release the biological molecules (e.g., locked in hydrogel). The rolling circle amplification products may be cleaved. The permeabilization step may facilitates the release of the RNA transcripts, the second probes or probe sets, and/or the ligation products from the sample to interact with the array of capture probes (e.g., after the in situ analysis). In some embodiments, an RNA comprising the first variant sequence analyzed using an in situ detection method is additionally captured and detected on the capture array. Once the capture probes capture RNA targets (e.g., mRNA transcripts from a sample that has been analyzed in an in situ assay disclosed herein), first strand cDNA created by template switching and reverse transcriptase is then denatured and the second strand is then extended. The second strand cDNA is then denatured from the first strand cDNA, neutralized, and transferred to a tube. cDNA quantification and amplification can be performed using standard techniques discussed herein. The cDNA can then be subjected to library preparation and indexing, including fragmentation, end-repair, A-tailing, and/or indexing PCR steps, followed by an optional library QC step.

In some embodiments, a method disclosed herein comprises detecting one or more other analytes in the biological sample, in addition to detecting a variant sequence disclosed herein. In some embodiments, the one or more other analytes comprise a nucleic acid analyte and/or a non-nucleic acid analyte such as a protein. The one or more other analytes can comprise any biomolecule or chemical compound, including a macromolecule such as a protein or peptide, a lipid or a nucleic acid molecule, or a small molecule, including organic or inorganic molecules. The one or more other analytes can be a cell or a microorganism, including a virus, or a fragment or product thereof. The one or more other analytes can be any substance or entity for which a specific binding partner (e.g. an affinity binding partner) can be developed. Such a specific binding partner may be a nucleic acid probe (for a nucleic acid analyte). Alternatively, the specific binding partner may be coupled to a nucleic acid. The one or more other analytes can include nucleic acid molecules, such as DNA (e.g., genomic DNA, mitochondrial DNA, plastid DNA, viral DNA, etc.) and RNA (e.g., mRNA, microRNA, rRNA, snRNA, viral RNA, etc.), and synthetic and/or modified nucleic acid molecules, (e.g., including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The one or more other analytes may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. The one or more other analytes may include intracellular analytes, such as proteins, protein modifications (e.g., phosphorylation status or other post-translational modifications), nuclear proteins, nuclear membrane proteins, or any combination thereof. The one or more other analytes can be a single molecule or a complex that contains two or more molecular subunits, e.g., including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g., interactions between proteins and nucleic acids, e.g. regulatory factors, such as transcription factors, and DNA or RNA.

Any of the additional analytes described herein, including those exemplified in the preceding paragraph, may be analyzed using an in situ analysis (e.g., using fluorescent microscopy or in situ sequencing as a read out) and/or using an array of capture probes (e.g., using NGS sequencing of spatially barcoded nucleic acid molecules comprising analyte sequences). Optical signals and/or spatially barcoded nucleic acid molecules associated with a variant sequence of interest and those associated with one or more of the additional analytes (e.g., a different variant sequence of the same gene, a variant sequence of a different gene, or an mRNA transcript of the same gene or a different gene) can be generated, detected, and/or analyzed simultaneously or sequentially in any suitable order.

Exemplary steps for sample preparation, permeabilization, DNA generation (e.g., first strand cDNA generation and second strand generation), DNA amplification (e.g., cDNA amplification) and quality control, and spatial gene expression library construction are disclosed for example in US 2021/0317524, US 2021/0332424, US 2021/0317524. US 2021/0324457, US 2021/0332425, all of which are incorporated herein by reference in their entireties.

### A. Capture Probes

A capture probe herein can comprise any molecule capable of capturing (directly or indirectly) and/or labelling an analyte of interest in a biological sample (e.g., a ligation product). In some embodiments, the ligation product for capture comprises a ligated second probe or probe set as described in Section V.A. In some embodiments, the capture probe is a nucleic acid. In some embodiments, the capture probe is a conjugate (e.g., an oligonucleotide-antibody conjugate). In some embodiments, the capture probe includes a barcode (e.g., a spatial barcode and/or a unique molecular identifier (UMI)) and a capture domain.

In some embodiments, analytes in a biological sample are pre-processed prior to interaction with a capture probe. For example, prior to interaction with capture probes, polymerization reactions catalyzed by a polymerase (e.g., DNA polymerase or reverse transcriptase) are performed in the biological sample. In some embodiments, a primer for the polymerization reaction includes a functional group that enhances hybridization with the capture probe. The capture probes can include appropriate capture domains to capture biological analytes of interest (e.g., the ligation products disclosed herein and/or mRNA transcripts).

In some aspects, a reverse transcriptase (RT) catalyzed reaction may take place during hybridization of one or more nucleic acid probes to a nucleic acid target in a biological sample, e.g., for an in situ assay and/or a spatial assay. In some embodiments, the RT reaction converts sequence information in one or more RNA analytes (e.g., mRNA transcripts comprising variant sequences such as SNPs or point mutations) in the biological sample to DNA sequences in probe molecules (e.g., a circularized gap-filled probe generated from a gap-filled first probe or probe set) for the in situ assay and/or a spatial assay. In some embodiments, the one or more nucleic acid probes comprise a probe that is ligated with another probe or to itself. For example, a circularizable probe can be ligated using RNA-templated and/or DNA-templated ligation.

In some embodiments, a reverse transcriptase (RT) catalyzed reaction takes place after ligation of a nucleic acid probe with another probe or to itself, wherein the nucleic acid probe hybridizes to a nucleic acid target (e.g., mRNA transcripts comprising variant sequences such as SNPs or point mutations) in a biological sample for an in situ assay and/or a spatial assay. In some embodiments, the RT reaction converts sequence information in one or more RNA analytes in the biological sample to DNA sequences in probe molecules (e.g., a circularized gap-filled probe generated from a gap-filled first probe or probe set) for the in situ assay and/or a spatial assay.

In some embodiments, biological analytes are pre-processed for library generation via next generation sequencing. For example, analytes can be pre-processed by addition of a modification (e.g., ligation of sequences that allow interaction with capture probes). In some embodiments, analytes are fragmented using fragmentation techniques. Fragmentation can be followed by a modification of the analyte. For example, a modification can be the addition through ligation of an adapter sequence that allows hybridization with the capture probe. In some embodiments, where the analyte of interest is RNA (e.g., mRNA), poly(A) tailing is performed. Addition of a poly(A) tail to RNA that does not contain a poly(A) tail can facilitate hybridization with a capture probe that includes a capture domain with a functional amount of poly(dT) sequence.

In some embodiments, prior to interaction with capture probes, ligation reactions catalyzed by a ligase are performed in the biological sample. In some embodiments, ligation is performed by chemical ligation. In some embodiments, the ligation is performed using click chemistry as further below. In some embodiments, the capture domain includes a DNA sequence that has complementarity to a RNA molecule, where the RNA molecule has complementarity to a second DNA sequence, and where the RNA-DNA sequence complementarity is used to ligate the second DNA sequence to the DNA sequence in the capture domain. In these embodiments, direct detection of RNA molecules is possible.

In some embodiments, prior to interaction with capture probes, target-specific reactions are performed in the biological sample. Examples of target specific reactions include, but are not limited to, ligation of target specific adaptors, probes and/or other oligonucleotides, target specific amplification using primers specific to one or more analytes, and target-specific detection using in situ hybridization, and microscopy. In some embodiments, a capture probe includes capture domains targeted to target-specific products (e.g., amplification or ligation).

In some embodiments, the capture probes may comprise one or more cleavable capture probes, wherein the cleaved capture probe can enter into a non-permeabilized cell and bind to target analytes within the sample. The capture probe may contain a cleavage domain, a cell penetrating peptide, a reporter molecule, and a disulfide bond (—S—S—). In some cases, the capture probe may also include a spatial barcode and a capture domain.

i. Capture Domain

In some embodiments, each capture agent (e.g., a capture probe) comprises at least one capture domain, which may comprise an oligonucleotide that binds specifically to a desired analyte. In some embodiments, a capture domain can be used to capture or detect a desired analyte, such as a ligation product disclosed herein or an mRNA.

In some embodiments, the capture domain comprises a functional nucleic acid sequence configured to interact with one or more analytes, such as one or more different ligation products of different second probes or probe sets that correspond to different variant sequences of interest. In some embodiments, the functional nucleic acid sequence can include an N-mer sequence (e.g., a random N-mer sequence), which N-mer sequences are configured to interact with a plurality of nucleic acid molecules, including RNA and/or DNA molecules. In some embodiments, the functional sequence can include a poly(T) sequence, which poly(T) sequences are configured to interact with messenger RNA (mRNA) molecules via the poly(A) tail of an mRNA transcript.

In some embodiments, capture probes include ribonucleotides and/or deoxyribonucleotides as well as synthetic nucleotide residues that are capable of participating in Watson-Crick type or analogous base pair interactions. In some embodiments, the capture domain is capable of priming a reverse transcription reaction to generate cDNA that is complementary to the captured RNA molecules (e.g., mRNA). In some embodiments, the capture domain of the capture probe can prime a DNA extension (polymerase) reaction to generate DNA that is complementary to the captured DNA molecules. In some embodiments, the capture domain can template a ligation reaction between the captured DNA molecules and a surface probe that is directly or indirectly immobilized on the substrate. In some embodiments, the capture domain can be ligated to one strand of the captured DNA molecules. For example, SplintR® ligase along with RNA or DNA sequences (e.g., degenerate RNA) can be used to ligate a single-stranded DNA or RNA to the capture domain. In some embodiments, ligases with RNA-templated ligase activity, e.g., SplintR® ligase, T4 RNA ligase 2 or KOD ligase, can be used to ligate a single-stranded DNA or RNA to the capture domain. In some embodiments, a capture domain includes a splint oligonucleotide. In some embodiments, a capture domain captures a splint oligonucleotide.

In some embodiments, the capture domain is located at the 3′ end of the capture probe and includes a free 3′ end that can be extended, e.g. by template dependent polymerization, to form an extended capture probe as described herein. In some embodiments, the capture domain includes a nucleotide sequence that is capable of hybridizing to nucleic acid, e.g. a ligation product or an mRNA, present in the cells of the tissue sample contacted with the array. In some embodiments, the capture domain can be selected or designed to bind selectively or specifically to a target nucleic acid (e.g., a ligation product or an RNA). For example, the capture domain can be selected or designed to capture mRNA by way of hybridization to the mRNA poly(A) tail. Thus, in some embodiments, the capture domain includes a poly(T) DNA oligonucleotide, e.g., a series of consecutive deoxythymidine residues linked by phosphodiester bonds, which is capable of hybridizing to the poly(A) tail of mRNA. In some embodiments, the capture domain can include nucleotides that are functionally or structurally analogous to a poly(T) tail. For example, a poly(U) oligonucleotide or an oligonucleotide included of deoxythymidine analogues. In some embodiments, the capture domain includes at least 10, 11, 12, 13, 14, 15, 16, 17, 18, 19, or 20 nucleotides. In some embodiments, the capture domain includes at least 25, 30, or 35 nucleotides.

In some embodiments, random sequences, e.g., random hexamers or similar sequences, can be used to form all or a part of the capture domain. For example, random sequences can be used in conjunction with poly(T) (or poly(T) analogue) sequences. Thus, where a capture domain includes a poly(T) (or a “poly(T)-like”) oligonucleotide, it can also include a random oligonucleotide sequence (e.g., “poly(T)-random sequence” probe). This can, for example, be located 5′ or 3′ of the poly(T) sequence, e.g. at the 3′ end of the capture domain. The poly(T)-random sequence probe can facilitate the capture of the mRNA poly(A) tail. In some embodiments, the capture domain can be an entirely random sequence. In some embodiments, degenerate capture domains can be used.

In some embodiments, a pool of two or more capture probes form a mixture, where the capture domain of one or more capture probes includes a poly(T) sequence and the capture domain of one or more capture probes includes random sequences. In some embodiments, a pool of two or more capture probes form a mixture where the capture domain of one or more capture probes includes poly(T)-like sequence and the capture domain of one or more capture probes includes random sequences. In some embodiments, a pool of two or more capture probes form a mixture where the capture domain of one or more capture probes includes a poly(T)-random sequences and the capture domain of one or more capture probes includes random sequences. In some embodiments, probes with degenerate capture domains can be added to any of the preceding combinations listed herein. In some embodiments, probes with degenerate capture domains can be substituted for one of the probes in each of the pairs described herein.

The capture domain can be based on a particular gene sequence or particular motif sequence or common/conserved sequence, that it is designed to capture (e.g., a sequence-specific capture domain). Thus, in some embodiments, the capture domain is capable of binding selectively to a desired sub-type or subset of nucleic acid. In some embodiments, the capture domain is capable of binding selectively to one or more ligation products disclosed herein.

In some embodiments, a capture domain includes an “anchor” or “anchoring sequence”, which is a sequence of nucleotides that is designed to ensure that the capture domain hybridizes to the intended biological analyte. In some embodiments, an anchor sequence includes a sequence of nucleotides, including a 1-mer, 2-mer, 3-mer or longer sequence. In some embodiments, the short sequence is random. For example, a capture domain including a poly(T) sequence can be designed to capture an mRNA. In such embodiments, an anchoring sequence can include a random 3-mer (e.g., GGG) that helps ensure that the poly(T) capture domain hybridizes to an mRNA. In some embodiments, an anchoring sequence can be VN, N, or NN. Alternatively, the sequence can be designed using a specific sequence of nucleotides. In some embodiments, the anchor sequence is at the 3′ end of the capture domain. In some embodiments, the anchor sequence is at the 5′ end of the capture domain.

ii. Cleavage Domain

Each capture probe can optionally include at least one cleavage domain. The cleavage domain represents the portion of the probe that is used to reversibly attach the probe to an array feature, as will be described further below. Further, one or more segments or regions of the capture probe can optionally be released from the array feature by cleavage of the cleavage domain. As an example spatial barcodes and/or universal molecular identifiers (UMIs) can be released by cleavage of the cleavage domain.

In some embodiments, the cleavage domain linking the capture probe to a feature is a disulfide bond. A reducing agent can be added to break the disulfide bonds, resulting in release of the capture probe from the feature. As another example, heating can also result in degradation of the cleavage domain and release of the attached capture probe from the array feature. In some embodiments, laser radiation is used to heat and degrade cleavage domains of capture probes at specific locations. In some embodiments, the cleavage domain is a photo-sensitive chemical bond (e.g., a chemical bond that dissociates when exposed to light such as ultraviolet light).

Other examples of cleavage domains include labile chemical bonds such as, but not limited to, ester linkages (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)).

In some embodiments, the cleavage domain includes a sequence that is recognized by one or more enzymes capable of cleaving a nucleic acid molecule, e.g., capable of breaking the phosphodiester linkage between two or more nucleotides. A bond can be cleavable via other nucleic acid molecule targeting enzymes, such as restriction enzymes (e.g., restriction endonucleases). For example, the cleavage domain can include a restriction endonuclease (restriction enzyme) recognition sequence. Restriction enzymes cut double-stranded or single stranded DNA at specific recognition nucleotide sequences known as restriction sites. In some embodiments, a rare-cutting restriction enzyme, e.g., enzymes with a long recognition site (at least 8 base pairs in length), is used to reduce the possibility of cleaving elsewhere in the capture probe.

In some embodiments, the cleavage domain includes a poly(U) sequence which can be cleaved by a mixture of Uracil DNA glycosylase (UDG) and the DNA glycosylase-lyase Endonuclease VIII, commercially known as the USER™ enzyme. Releasable capture probes can be available for reaction once released. Thus, for example, an activatable capture probe can be activated by releasing the capture probes from a feature.

In some embodiments, where the capture probe is attached indirectly to a substrate, e.g., via a surface probe, the cleavage domain includes one or more mismatch nucleotides, so that the complementary parts of the surface probe and the capture probe are not 100% complementary (for example, the number of mismatched base pairs can one, two, or three base pairs). Such a mismatch is recognized, e.g., by the MutY and T7 endonuclease I enzymes, which results in cleavage of the nucleic acid molecule at the position of the mismatch.

In some embodiments, where the capture probe is attached to a feature indirectly, e.g., via a surface probe, the cleavage domain includes a nickase recognition site or sequence. Nickases are endonucleases which cleave only a single strand of a DNA duplex. Thus, the cleavage domain can include a nickase recognition site close to the 5′ end of the surface probe (and/or the 5′ end of the capture probe) such that cleavage of the surface probe or capture probe destabilizes the duplex between the surface probe and capture probe thereby releasing the capture probe) from the feature.

Nickase enzymes can also be used in some embodiments where the capture probe is attached to the feature directly. For example, the substrate can be contacted with a nucleic acid molecule that hybridizes to the cleavage domain of the capture probe to provide or reconstitute a nickase recognition site, e.g., a cleavage helper probe. Thus, contact with a nickase enzyme will result in cleavage of the cleavage domain thereby releasing the capture probe from the feature. Such cleavage helper probes can also be used to provide or reconstitute cleavage recognition sites for other cleavage enzymes, e.g., restriction enzymes.

Some nickases introduce single-stranded nicks only at particular sites on a DNA molecule, by binding to and recognizing a particular nucleotide recognition sequence. A number of naturally-occurring nickases have been discovered, of which at present the sequence recognition properties have been determined for at least four. Nickases are described in U.S. Pat. No. 6,867,028, which is incorporated herein by reference in its entirety. In general, any suitable nickase can be used to bind to a complementary nickase recognition site of a cleavage domain. Following use, the nickase enzyme can be removed from the assay or inactivated following release of the capture probes to prevent unwanted cleavage of the capture probes.

Examples of suitable capture domains that are not exclusively nucleic-acid based include, but are not limited to, proteins, peptides, aptamers, antigens, antibodies, and molecular analogs that mimic the functionality of any of the capture domains described herein.

In some embodiments, a cleavage domain is absent from the capture probe. Examples of substrates with attached capture probes lacking a cleavage domain are described for example in Macosko et al., (2015) Cell 161, 1202-1214, the entire contents of which are incorporated herein by reference.

In some embodiments, the region of the capture probe corresponding to the cleavage domain can be used for some other function. For example, an additional region for nucleic acid extension or amplification can be included where the cleavage domain would normally be positioned. In such embodiments, the region can supplement the functional domain or even exist as an additional functional domain. In some embodiments, the cleavage domain is present but its use is optional.

iii. Functional Domain

Each capture probe can optionally include at least one functional domain. Each functional domain typically includes a functional nucleotide sequence for a downstream analytical step in the overall analysis procedure.

In some cases, the nucleic acid molecule (e.g., capture probe) can comprise one or more functional sequences. For example, a functional sequence can comprise a sequence for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the functional sequence can comprise a barcode sequence or multiple barcode sequences. In some cases, the functional sequence can comprise a unique molecular identifier (UMI). In some cases, the functional sequence can comprise a primer sequence (e.g., an R1 primer sequence for Illumina sequencing, an R2 primer sequence for Illumina sequencing, etc.). In some cases, a functional sequence can comprise a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence may contain a contiguous or continuous portion or segment, but not all, of a full sequence, for example. In some cases, a downstream procedure may extend the partial sequence, or derivative thereof, to achieve a full sequence of the partial sequence, or derivative thereof. Examples of such capture probes and uses thereof are described in U.S. Patent Publication Nos. 2014/0378345 and 2015/0376609, the entire contents of each of which are incorporated herein by reference. The functional domains can be selected for compatibility with a variety of different sequencing systems, e.g., 454 Sequencing, Ion Torrent Proton or PGM, Illumina X10, etc., or other platforms from Illumina, BGI, Qiagen, Thermo-Fisher, PacBio, and Roche, and the requirements thereof.

iv. Spatial Barcode

As discussed above, the capture probe can include one or more spatial barcodes (e.g., two or more, three or more, four or more, five or more) spatial barcodes. A “spatial barcode” is a contiguous nucleic acid segment or two or more non-contiguous nucleic acid segments that function as a label or identifier that conveys or is capable of conveying spatial information. In some embodiments, a capture probe includes a spatial barcode that possesses a spatial aspect, where the barcode is associated with a particular location within an array or a particular location on a substrate. Exemplary spatial barcodes are described in U.S. Pat. No. 10,030,261, which is incorporated herein by reference.

A spatial barcode can be part of a capture probe. A spatial barcode can be unique. In some embodiments where the spatial barcode is unique, the spatial barcode functions both as a spatial barcode and as a unique molecular identifier (UMI), associated with one particular capture probe.

Spatial barcodes can have a variety of different formats. For example, spatial barcodes can include polynucleotide spatial barcodes; random nucleic acid and/or amino acid sequences; and synthetic nucleic acid and/or amino acid sequences. In some embodiments, a spatial barcode is attached to an analyte in a reversible or irreversible manner. In some embodiments, a spatial barcode allows for identification and/or quantification of individual sequencing-reads. In some embodiments, a spatial barcode is a used as a barcode for which fluorescently labeled oligonucleotide probes hybridize to the spatial barcode.

In some embodiments, the spatial barcode is a nucleic acid sequence that does not substantially hybridize to nucleic acid molecules such as mRNAs in a biological sample. In some embodiments, the spatial barcode has less than 80% sequence identity (e.g., less than 70%, 60%, 50%, or less than 40% sequence identity) to the nucleic acid molecules such as mRNAs across a substantial part (e.g., 80% or more) of the nucleic acid molecules in the biological sample.

The spatial barcode sequences can include from about 6 to about 20 or more nucleotides within the sequence of the capture probes. In some embodiments, the length of a spatial barcode sequence can be about 6, 7, 8, 9, 10, 11, 12, 13, 14, 15, 16, 17, 18, 19, 20 nucleotides or longer. In some embodiments, the length of a spatial barcode sequence can be at least about 6, 7, 8, 9, 10, 11, 12, 13, 14, 15, 16, 17, 18, 19, 20 nucleotides or longer. In some embodiments, the length of a spatial barcode sequence is at most about 6, 7, 8, 9, 10, 11, 12, 13, 14, 15, 16, 17, 18, 19, 20 nucleotides or shorter. These nucleotides can be completely contiguous, e.g., in a single stretch of adjacent nucleotides, or they can be separated into two or more separate subsequences that are separated by 1 or more nucleotides. Separated spatial barcode subsequences can be from about 4 to about 16 nucleotides in length. In some embodiments, the spatial barcode subsequence can be about 4, 5, 6, 7, 8, 9, 10, 11, 12, 13, 14, 15, 16 nucleotides or longer. In some embodiments, the spatial barcode subsequence can be at least about 4, 5, 6, 7, 8, 9, 10, 11, 12, 13, 14, 15, 16 nucleotides or longer. In some embodiments, the spatial barcode subsequence can be at most about 4, 5, 6, 7, 8, 9, 10, 11, 12, 13, 14, 15, 16 nucleotides or shorter.

For multiple capture probes that are attached to a common array feature, the one or more spatial barcode sequences of the multiple capture probes can include sequences that are the same for all capture probes coupled to the feature, and/or sequences that are different across all capture probes coupled to the feature. In some embodiments, a plurality of capture probes attached to a common array feature may possess the same spatial barcode, but have different capture domains designed to associate the spatial barcode of the feature with more than one target analyte. For example, a feature may be coupled to two, three, four, five, six, seven, eight, nine, ten, or more different types of spatially-barcoded capture probes, each type of spatially-barcoded capture probe possessing the spatial barcode. In some aspects, capture-probe barcoded constructs can be tailored for analyses of any given analyte associated with a nucleic acid and capable of binding with such a construct. In some embodiments, the analyte is a ligation product capable of binding with a spatially-barcoded capture probe disclosed herein. In some embodiments, the analyte is a genomic DNA, a cellular RNA (e.g., mRNA), or a cDNA capable of binding with a spatially-barcoded capture probe disclosed herein.

Capture probes attached to a single array feature can include identical (or common) spatial barcode sequences, different spatial barcode sequences, or a combination of both. Capture probes attached to a feature can include multiple sets of capture probes. Capture probes of a given set can include identical spatial barcode sequences. The identical spatial barcode sequences can be different from spatial barcode sequences of capture probes of another set.

The plurality of capture probes can include spatial barcode sequences that are associated with specific locations on a spatial array. For example, a first plurality of capture probes can be associated with a first region, based on a spatial barcode sequence common to the capture probes within the first region, and a second plurality of capture probes can be associated with a second region, based on a spatial barcode sequence common to the capture probes within the second region. The second region may or may not be associated with the first region. Additional pluralities of capture probes can be associated with spatial barcode sequences common to the capture probes within other regions. In some embodiments, the spatial barcode sequences can be the same across a plurality of capture probe molecules.

In some embodiments, multiple different spatial barcodes are incorporated into a single arrayed capture probe. For example, a mixed but known set of spatial barcode sequences can provide a stronger address or attribution of the spatial barcodes to a given spot or location, by providing duplicate or independent confirmation of the identity of the location. In some embodiments, the multiple spatial barcodes represent increasing specificity of the location of the particular array point.

v. Unique Molecular Identifier

The capture probe can include one or more (e.g., two or more, three or more, four or more, five or more) Unique Molecular Identifiers (UMIs). A unique molecular identifier is a contiguous nucleic acid segment or two or more non-contiguous nucleic acid segments that function as a label or identifier for a particular analyte, or for a capture probe that binds a particular analyte (e.g., via the capture domain).

A UMI can be unique. A UMI can include one or more specific polynucleotides sequences, one or more random nucleic acid and/or amino acid sequences, and/or one or more synthetic nucleic acid and/or amino acid sequences.

In some embodiments, the UMI is a nucleic acid sequence that does not substantially hybridize to analyte nucleic acid molecules in a biological sample. In some embodiments, the UMI has less than 80% sequence identity (e.g., less than 70%, 60%, 50%, or less than 40% sequence identity) to the nucleic acid sequences across a substantial part (e.g., 80% or more) of the nucleic acid molecules in the biological sample.

The UMI can include from about 6 to about 20 or more nucleotides within the sequence of the capture probes. In some embodiments, the length of a UMI sequence can be about 6, 7, 8, 9, 10, 11, 12, 13, 14, 15, 16, 17, 18, 19, 20 nucleotides or longer. In some embodiments, the length of a UMI sequence can be at least about 6, 7, 8, 9, 10, 11, 12, 13, 14, 15, 16, 17, 18, 19, 20 nucleotides or longer. In some embodiments, the length of a UMI sequence is at most about 6, 7, 8, 9, 10, 11, 12, 13, 14, 15, 16, 17, 18, 19, 20 nucleotides or shorter. These nucleotides can be completely contiguous, e.g., in a single stretch of adjacent nucleotides, or they can be separated into two or more separate subsequences that are separated by 1 or more nucleotides. Separated UMI subsequences can be from about 4 to about 16 nucleotides in length. In some embodiments, the UMI subsequence can be about 4, 5, 6, 7, 8, 9, 10, 11, 12, 13, 14, 15, 16 nucleotides or longer. In some embodiments, the UMI subsequence can be at least about 4, 5, 6, 7, 8, 9, 10, 11, 12, 13, 14, 15, 16 nucleotides or longer. In some embodiments, the UMI subsequence can be at most about 4, 5, 6, 7, 8, 9, 10, 11, 12, 13, 14, 15, 16 nucleotides or shorter.

In some embodiments, a UMI is attached to an analyte in a reversible or irreversible manner. In some embodiments, a UMI allows for identification and/or quantification of individual sequencing-reads. In some embodiments, a UMI is a used as a fluorescent barcode for which fluorescently labeled oligonucleotide probes hybridize to the UMI.

vi. Other Aspects of Capture Probes

For capture probes that are attached to an array feature, an individual array feature can include one or more capture probes. In some embodiments, an individual array feature includes hundreds or thousands of capture probes. In some embodiments, the capture probes are associated with a particular individual feature, where the individual feature contains a capture probe including a spatial barcode unique to a defined region or location on the array.

In some embodiments, a particular feature can contain capture probes including more than one spatial barcode (e.g., one capture probe at a particular feature can include a spatial barcode that is different than the spatial barcode included in another capture probe at the same particular feature, while both capture probes include a second, common spatial barcode), where each spatial barcode corresponds to a particular defined region or location on the array. For example, multiple spatial barcode sequences associated with one particular feature on an array can provide a stronger address or attribution to a given location by providing duplicate or independent confirmation of the location. In some embodiments, the multiple spatial barcodes represent increasing specificity of the location of the particular array point. In a non-limiting example, a particular array point can be coded with two different spatial barcodes, where each spatial barcode identifies a particular defined region within the array, and an array point possessing both spatial barcodes identifies the sub-region where two defined regions overlap, e.g., such as the overlapping portion of a Venn diagram.

In another non-limiting example, a particular array point can be coded with three different spatial barcodes, where the first spatial barcode identifies a first region within the array, the second spatial barcode identifies a second region, where the second region is a subregion entirely within the first region, and the third spatial barcode identifies a third region, where the third region is a subregion entirely within the first and second subregions.

In some embodiments, capture probes attached to array features are released from the array features for sequencing. Alternatively, in some embodiments, capture probes remain attached to the array features, and the probes are sequenced while remaining attached to the array features. Further aspects of the sequencing of capture probes are described in subsequent sections of this disclosure.

In some embodiments, an array feature can include different types of capture probes attached to the feature. For example, the array feature can include a first type of capture probe with a capture domain designed to bind to one type of analyte, and a second type of capture probe with a capture domain designed to bind to a second type of analyte. In general, array features can include one or more (e.g., two or more, three or more, four or more, five or more, six or more, eight or more, ten or more, 12 or more, 15 or more, 20 or more, 30 or more, 50 or more) different types of capture probes attached to a single array feature.

In some embodiments, the capture probe is nucleic acid. In some embodiments, the capture probe is attached to the array feature via its 5′ end. In some embodiments, the capture probe includes from the 5′ to 3′ end: one or more barcodes (e.g., a spatial barcode and/or a UMI) and one or more capture domains. In some embodiments, the capture probe includes from the 5′ to 3′ end: one barcode (e.g., a spatial barcode or a UMI) and one capture domain. In some embodiments, the capture probe includes from the 5′ to 3′ end: a cleavage domain, a functional domain, one or more barcodes (e.g., a spatial barcode and/or a UMI), and a capture domain. In some embodiments, the capture probe includes from the 5′ to 3′ end: a cleavage domain, a functional domain, one or more barcodes (e.g., a spatial barcode and/or a UMI), a second functional domain, and a capture domain. In some embodiments, the capture probe includes from the 5′ to 3′ end: a cleavage domain, a functional domain, a spatial barcode, a UMI, and a capture domain. In some embodiments, the capture probe does not include a spatial barcode. In some embodiments, the capture probe does not include a UMI. In some embodiments, the capture probe includes a sequence for initiating a sequencing reaction.

In some embodiments, the capture probe is immobilized on a feature via its 3′ end. In some instances, the capture probe comprises: an adapter sequence—a barcode (e.g., a spatial barcode) —an optional unique molecular identifier (UMI) sequence—a capture domain. In some embodiments, the capture probe includes from the 3′ to 5′ end: one or more barcodes (e.g., a spatial barcode and/or a UMI) and one or more capture domains. In some embodiments, the capture probe includes from the 3′ to 5′ end: one barcode (e.g., a spatial barcode or a UMI) and one capture domain. In some embodiments, the capture probe includes from the 3′ to 5′ end: a cleavage domain, a functional domain, one or more barcodes (e.g., a spatial barcode and/or a UMI), and a capture domain. In some embodiments, the capture probe includes from the 3′ to 5′ end: a cleavage domain, a functional domain, a spatial barcode, a UMI, and a capture domain.

In some embodiments, a capture probe includes an in situ synthesized oligonucleotide. In some embodiments, the in situ synthesized oligonucleotide includes one or more constant sequences, one or more of which serves as a priming sequence (e.g., a primer for amplifying target nucleic acids). In some embodiments, a constant sequence is a cleavable sequence. In some embodiments, the in situ synthesized oligonucleotide includes a barcode sequence, e.g., a variable barcode sequence. In some embodiments, the in situ synthesized oligonucleotide is attached to a feature of an array.

In some embodiments, a capture probe is a product of two or more oligonucleotide sequences, e.g., two or more oligonucleotide sequences that are ligated together. In some embodiments, one of the oligonucleotide sequences is an in situ synthesized oligonucleotide.

In some embodiments, the capture probe includes a splint oligonucleotide. Two or more oligonucleotides can be ligated together using a splint oligonucleotide and any variety of suitable ligases described herein (e.g., a SplintR® ligase).

In some embodiments, one of the oligonucleotides includes: a constant sequence (e.g., a sequence complementary to a portion of a splint oligonucleotide), a degenerate sequence, and a capture domain (e.g., as described herein). In some embodiments, the capture probe is generated by having an enzyme add polynucleotides at the end of an oligonucleotide sequence. The capture probe can include a degenerate sequence, which can function as a unique molecular identifier.

A capture probe can include a degenerate sequence, which is a sequence in which some positions of a nucleotide sequence contain a number of possible bases. A degenerate sequence can be a degenerate nucleotide sequence including about or at least 5, 6, 7, 8, 9, 10, 11, 12, 13, 14, 15, 16, 17, 18, 19, 20, 25, 30, 35, 40, 45, or 50 nucleotides. In some embodiments, a nucleotide sequence contains 1, 2, 3, 4, 5, 6, 7, 8, 9, 0, 10, 15, 20, 25, or more degenerate positions within the nucleotide sequence. In some embodiments, the degenerate sequence is used as a UMI.

In some embodiments, a capture probe includes a restriction endonuclease recognition sequence or a sequence of nucleotides cleavable by specific enzyme activities. For example, uracil sequences can be cleaved by specific enzyme activity. As another example, other modified bases (e.g., modified by methylation) can be recognized and cleaved by specific endonucleases. The capture probes can be subjected to an enzymatic cleavage, which removes the blocking domain and any of the additional nucleotides that are added to the 3′ end of the capture probe during the modification process. The removal of the blocking domain reveals and/or restores the free 3′ end of the capture domain of the capture probe. In some embodiments, additional nucleotides can be removed to reveal and/or restore the 3′ end of the capture domain of the capture probe.

In some embodiments, a blocking domain can be incorporated into the capture probe when it is synthesized, or after its synthesis. The terminal nucleotide of the capture domain is a reversible terminator nucleotide (e.g., 3′-O-blocked reversible terminator and 3′-unblocked reversible terminator), and can be included in the capture probe during or after probe synthesis.

vii. Extended Capture Probes

An “extended capture probe” is a capture probe with an enlarged nucleic acid sequence. For example, where the capture probe includes nucleic acid, an “extended 3′ end” indicates that further nucleotides were added to the most 3′ nucleotide of the capture probe to extend the length of the capture probe, for example, by standard polymerization reactions utilized to extend nucleic acid molecules including templated polymerization catalyzed by a polymerase (e.g., a DNA polymerase or reverse transcriptase).

In some embodiments, extending the capture probe includes generating cDNA from the captured (hybridized) RNA. This process involves synthesis of a complementary strand of the hybridized nucleic acid, e.g., generating cDNA based on the captured RNA template (the RNA hybridized to the capture domain of the capture probe). Thus, in an initial step of extending the capture probe, e.g., the cDNA generation, the captured (hybridized) nucleic acid, e.g., RNA, acts as a template for the extension, e.g., reverse transcription, step.

In some embodiments, the capture probe is extended using reverse transcription. For example, reverse transcription includes synthesizing cDNA (complementary or copy DNA) from RNA, e.g., (messenger RNA), using a reverse transcriptase. In some embodiments, reverse transcription is performed while the tissue is still in place, generating an analyte library, where the analyte library includes the spatial barcodes from the adjacent capture probes. In some embodiments, the capture probe is extended using one or more DNA polymerases.

In some embodiments, the capture domain of the capture probe includes a primer for producing the complementary strand of the nucleic acid hybridized to the capture probe, e.g., a primer for DNA polymerase and/or reverse transcription. The nucleic acid, e.g., DNA and/or cDNA, molecules generated by the extension reaction incorporate the sequence of the capture probe. The extension of the capture probe, e.g., a DNA polymerase and/or reverse transcription reaction, can be performed using a variety of suitable enzymes and protocols.

For example, the capture sequences in the ligation products of the second probes or probe sets can be captured onto the capture array slide. In some embodiments, capture probes with capture domains can bind to capture sequences on ligation products or complements thereof disclosed herein. In some embodiments, one or more reactions (e.g., extension, and/or ligation) are performed to generate a spatially labeled polynucleotide sequence comprising a sequence of the ligation product or complement thereof and a sequence of the spatial barcode or complement thereof.

In some embodiments, a full-length DNA molecule is generated. In some embodiments, a “full-length” DNA molecule refers to the whole of the captured nucleic acid molecule (e.g., a ligation product disclosed herein). However, if the nucleic acid, e.g., RNA, was partially degraded in the tissue sample, then the captured nucleic acid molecules will not be the same length as the initial RNA in the tissue sample. In some embodiments, the 3′ end of the extended probes, e.g., first strand cDNA molecules, is modified. For example, a linker or adaptor can be ligated to the 3′ end of the extended probes. This can be achieved using single stranded ligation enzymes such as T4 RNA ligase or Circligase™ (available from Epicentre Biotechnologies, Madison, WI). In some embodiments, template switching oligonucleotides are used to extend cDNA in order to generate a full-length cDNA (or as close to a full-length cDNA as possible). In some embodiments, a second strand synthesis helper probe (a partially double stranded DNA molecule capable of hybridizing to the 3′ end of the extended capture probe), can be ligated to the 3′ end of the extended probe, e.g., first strand cDNA, molecule using a double stranded ligation enzyme such as T4 DNA ligase. Any suitable enzymes appropriate for the ligation step may be used and include, e.g., Tth DNA ligase, Taq DNA ligase, Thermococcus sp. (strain 9°N) DNA ligase (9°N™ DNA ligase, New England Biolabs), Ampligase™ (available from Epicentre Biotechnologies, Madison, WI), and SplintR® (available from New England Biolabs, Ipswich, MA). In some embodiments, a polynucleotide tail, e.g., a poly(A) tail, is incorporated at the 3′ end of the extended probe molecules. In some embodiments, the polynucleotide tail is incorporated using a terminal transferase active enzyme.

In some embodiments, double-stranded extended capture probes are treated to remove any unextended capture probes prior to amplification and/or analysis, e.g. sequence analysis. This can be achieved by a variety of methods, e.g., using an enzyme to degrade the unextended probes, such as an exonuclease enzyme, or purification columns.

In some embodiments, extended capture probes are amplified to yield quantities that are sufficient for analysis, e.g., via DNA sequencing. In some embodiments, the first strand of the extended capture probes (e.g., DNA and/or cDNA molecules) acts as a template for the amplification reaction (e.g., a polymerase chain reaction).

In some embodiments, the amplification reaction incorporates an affinity group onto the extended capture probe (e.g., RNA-cDNA hybrid) using a primer including the affinity group. In some embodiments, the primer includes an affinity group and the extended capture probes includes the affinity group. The affinity group can correspond to any of the affinity groups described previously.

In some embodiments, the extended capture probes including the affinity group can be coupled to an array feature specific for the affinity group. In some embodiments, the array feature includes avidin or streptavidin and the affinity group includes biotin. In some embodiments, the array feature includes maltose and the affinity group includes maltose-binding protein. In some embodiments, the array feature includes maltose-binding protein and the affinity group includes maltose. In some embodiments, amplifying the extended capture probes can function to release the extended probes from the array feature, insofar as copies of the extended probes are not attached to the array feature.

In some embodiments, the extended capture probe or complement or amplicon thereof is released from an array feature. The step of releasing the extended capture probe or complement or amplicon thereof from an array feature can be achieved in a number of ways. In some embodiments, an extended capture probe or a complement thereof is released from the feature by nucleic acid cleavage and/or by denaturation (e.g., by heating to denature a double-stranded molecule).

### B. Analysis of Captured Analytes

A wide variety of different sequencing methods can be used to analyze spatially barcoded oligonucleotides. In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog). In some embodiments, the spatially labeled polynucleotide (e.g., spatially barcoded oligonucleotide) a sequence of the ligation product or complement thereof generated from the second probe or probe set (e.g., as described in Section V) and (ii) a sequence of the spatial barcode or complement thereof.

Sequencing of polynucleotides can be performed by various commercial systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification.

Other examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and any combinations thereof.

In some embodiments, direct sequencing of one or more captured analytes is performed by sequencing-by-synthesis (SBS). In some embodiments, a sequencing primer is complementary to a sequence in one or more of the domains of a capture probe (e.g., functional domain). In such embodiments, sequencing-by-synthesis can include reverse transcription and/or amplification in order to generate a template sequence (e.g., functional domain) from which a primer sequence can bind.

SBS can involve hybridizing an appropriate primer, sometimes referred to as a sequencing primer, with the nucleic acid template to be sequenced, extending the primer, and detecting the nucleotides used to extend the primer. Preferably, the nucleic acid used to extend the primer is detected before a further nucleotide is added to the growing nucleic acid chain, thus allowing base-by-base nucleic acid sequencing. The detection of incorporated nucleotides is facilitated by including one or more labelled nucleotides in the primer extension reaction. To allow the hybridization of an appropriate sequencing primer to the nucleic acid template to be sequenced, the nucleic acid template should normally be in a single stranded form. If the nucleic acid templates making up the nucleic acid spots are present in a double stranded form these can be processed to provide single stranded nucleic acid templates using any suitable methods, for example by denaturation, cleavage etc. The sequencing primers which are hybridized to the nucleic acid template and used for primer extension are preferably short oligonucleotides, for example, 15 to 25 nucleotides in length. The sequencing primers can be provided in solution or in an immobilized form. Once the sequencing primer has been annealed to the nucleic acid template to be sequenced by subjecting the nucleic acid template and sequencing primer to appropriate conditions, primer extension is carried out, for example using a nucleic acid polymerase and a supply of nucleotides, at least some of which are provided in a labelled form, and conditions suitable for primer extension if a suitable nucleotide is provided. In some embodiments, a nucleic acid molecule or a complement thereof captured on the spatial array is released for sequencing. In some embodiments, the released nucleic acid molecule or complement thereof comprises the variant sequence. In some embodiments, the released nucleic acid molecule or complement thereof is a spatially labeled polynucleotide comprising (i) a sequence of a ligation product of a second probe set or complement thereof and (ii) a sequence of the spatial barcode or complement thereof.

### VII. Samples and Sample Processing

In some embodiments, a sample disclosed herein is derived from any biological sample. In some embodiments, the methods and compositions disclosed herein are used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. In some embodiments, a biological sample is obtained from non-mammalian organisms (e.g., a plant, an insect, an arachnid, a nematode, a fungus, or an amphibian). In some embodiments, a biological sample is obtained from a eukaryote, such as a tissue sample, a patient derived organoid (PDO) or patient derived xenograft (PDX). In some embodiments, a biological sample is from an organism and comprise one or more other organisms or components therefrom. For example, a mammalian tissue section may comprise a prion, a viroid, a virus, a bacterium, a fungus, or components from other organisms, in addition to mammalian cells and non-cellular tissue components. In some embodiments, subjects from which biological samples are obtained re healthy or asymptomatic individuals, individuals that have or are suspected of having a disease (e.g., a patient with a disease such as cancer) or a pre-disposition to a disease, and/or individuals in need of therapy or suspected of needing therapy.

In some embodiments, the biological sample include any number of macromolecules, for example, cellular macromolecules and organelles (e.g., mitochondria and nuclei). In some embodiments, the biological sample comprises nucleic acids (such as DNA or RNA), proteins/polypeptides, carbohydrates, and/or lipids. In some embodiments, the biological sample is obtained as a tissue sample, such as a tissue section, biopsy, a core biopsy, needle aspirate, or fine needle aspirate. In some embodiments, the biological sample is or comprise a cell pellet or a section of a cell pellet. In some embodiments, the biological sample is or comprise a cell block or a section of a cell block. In some embodiments, the sample is a fluid sample, such as a blood sample, urine sample, or saliva sample. In some embodiments, the sample comprises a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample comprises cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, a substrate herein can be any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In certain embodiments, the sample is attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate is coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Preparation

In some embodiments, a biological sample is harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section is prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. In some embodiments, the thickness of the tissue section is at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

In some embodiments, multiple sections are obtained from a single biological sample. For example, multiple tissue sections are obtained from a surgical biopsy sample by performing serial sectioning of the biopsy sample using a sectioning blade. In some embodiments, spatial information among the serial sections are preserved in this manner, and the sections can be analysed successively to obtain three-dimensional information about the biological sample.

In some embodiments, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample is prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample is prepared using formalin-fixation and paraffin-embedding (FFPE), which are established methods. In some embodiments, cell suspensions and other non-tissue samples are prepared using formalin-fixation and paraffin-embedding. Following fixation of the sample and embedding in a paraffin or resin block, the sample can be sectioned as described above. In some embodiments, prior to analysis, the paraffin-embedding material is removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes). In some embodiments, the biological sample (e.g., FFPE sample) is permeable after deparaffinization. In some embodiments, processing of the biological sample, such as de-waxing, allows the biological sample to become permeabilized.

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein.

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

In some embodiments, a biological sample is permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample is incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some embodiments, DNase and RNase inactivating agents or inhibitors such as proteinase K, and/or chelating agents such as EDTA, is added to the sample. For example, a method disclosed herein may comprise a step for increasing accessibility of a nucleic acid for binding, e.g., a denaturation step to open up DNA in a cell for hybridization by a probe. In some embodiments, proteinase K treatment is used to free up DNA with proteins bound thereto.

### (ii) Embedding

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample. Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some aspects, a biological sample can be embedded in any of a variety of other embedding materials to provide structural substrate to the sample prior to sectioning and other handling steps. In some cases, the embedding material is removed e.g., prior to analysis of tissue sections obtained from the sample. Suitable embedding materials include, but are not limited to, waxes, resins (e.g., methacrylate resins), epoxies, and agar.

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel comprises hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 m to about 2 mm.

Additional methods and aspects of hydrogel embedding of biological samples are described for example in Chen et al., Science 347(6221):543-548, 2015, the entire contents of which are incorporated herein by reference.

In some embodiments, the hydrogel forms the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible. In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some embodiments, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a biological sample embedded in a matrix (e.g., a hydrogel) is isometrically expanded. Isometric expansion methods that can be used include hydration, a preparative step in expansion microscopy, as described in, e.g., Chen et al., Science 347(6221):543-548, 2015 and U.S. Pat. No. 10,059,990, which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(iii) Staining and Immunohistochemistry (IHC)

To facilitate visualization, in some embodiments, biological samples are stained using a wide variety of stains and staining techniques. In some embodiments, for example, a sample is stained using any number of stains and/or immunohistochemical reagents. In some embodiments, one or more staining steps are performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain is specific to proteins, phospholipids, DNA (e.g., dsDNA, ssDNA), RNA, an organelle or compartment of the cell. In some embodiments, the sample is contacted with one or more labeled antibodies (e.g., a primary antibody specific for the analyte of interest and a labeled secondary antibody specific for the primary antibody). In some embodiments, cells in the sample is segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

In some embodiments, the sample is stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample is stained using Romanowsky stain, including Wright's stain, Jenner's stain, Can-Grunwald stain, Leishman stain, or Giemsa stain.

In some embodiments, biological samples is destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### VIII. Compositions, Kits, and Systems

Provided herein are compositions, systems or kits, for example comprising one or more oligonucleotides, e.g., any described in Sections I-VI, and instructions for performing the methods provided herein. In some embodiments, the systems or kits further comprise one or more reagents for performing the methods provided herein. In some embodiments, the systems or kits further comprise one or more reagents required for one or more steps comprising hybridization, ligation, extension, amplification, detection, and/or sample preparation as described herein. In some embodiments, the system or kit further comprises any one or more of the first probe or probe set, the second probe or probe set, and/or detectably labeled oligonucleotides disclosed herein, e.g., as described in Sections III and V. Provided herein are systems or kits, comprising a spatial array for detection of a ligation product of a second probe or probe set, e.g., a spatial array of capture probes as described in Section VI. In some embodiments, any or all of the oligonucleotides are DNA molecules. In some embodiments, the system or kit further comprises an enzyme such as a ligase and/or a polymerase described herein. In some embodiments, the ligase has DNA-splinted DNA ligase activity. In some embodiments, the system or kit comprises a polymerase, for instance for performing extension of the primers to incorporate modified nucleotides into cDNA products of transcripts. In some embodiments, the systems or kits may contain reagents for forming a functionalized matrix (e.g., a hydrogel), such as any suitable functional moieties. In some examples, also provided are buffers and reagents for tethering the modified primers, cDNA products, and/or RCA products to the functionalized matrix. The various components of the system or kit may be present in separate containers or certain compatible components may be pre-combined into a single container. In some embodiments, the systems or kits further contain instructions for using the components of the kit to practice the provided methods.

In some embodiments, provided herein is a system or kit for analyzing a biological sample, comprising: a) a circularizable probe comprising a first probe region and a second probe region that hybridize to a first target sequence and a second target sequence, respectively, in a target nucleic acid (e.g., RNA) in the biological sample, wherein the first and second target sequences are separated by a gap sequence in the target nucleic acid (e.g., RNA), and the gap sequence comprises a variant sequence among a plurality of different variant sequences; b) one or more reagents for circularizing the circularizable probe to generate a circularized probe comprising a gap-filled region complementary to the gap sequence; and/or c) one or more reagents for generating a rolling circle amplification product (RCP) of the circularized probe, wherein the RCP comprises multiple copies of the gap sequence.

In some embodiments, a system or kit disclosed herein comprises a pool of detection oligonucleotides each comprising a detectable label. In some embodiments, the biological sample is imaged to detect signals associated with the detectable labels at locations in the biological sample, thereby detecting one or more of the plurality of different variant sequences in the biological sample. In some embodiments, the one or more of the plurality of different variant sequences are identified (e.g., the identity of an SNP or point mutation is revealed) in the biological sample, based on the signals detected at the locations.

In some embodiments, a system or kit disclosed herein comprises a) a circularizable probe comprising a first probe region and a second probe region that hybridize to a first target sequence and a second target sequence, respectively, in a target nucleic acid in the biological sample, wherein the first and second target sequences are separated by a gap sequence in the target nucleic acid, and wherein the gap sequence comprises a variant sequence among a plurality of different variant sequences; and b) a library of splint oligonucleotides, wherein each splint oligonucleotide comprises: i) ligatable ends; and ii) a hybridization region complementary to one of the plurality of different variant sequences, wherein a splint oligonucleotide of the library of splint oligonucleotides that is complementary to the gap sequence is ligated to the circularizable probe, thereby circularizing the circularizable probe to generate a circularized probe comprising a gap-filled region complementary to the gap sequence. In some embodiments, each splint oligonucleotide of the library comprises a phosphate group on the 5′-end available for ligation.

The biological sample, the circularizable probe (e.g., in a composition comprising a plurality of circularizable probes), and the library of splint oligonucleotides can be contacted with one another in any order. For instance, the circularizable probe and the library of splint oligonucleotides can be pre-mixed prior to contacting the biological sample with the mixture. In other examples, the biological sample can be contacted with the circularizable probe and then with the library of splint oligonucleotides. In yet other examples, the biological sample can be contacted with the library of splint oligonucleotides and then with the circularizable probe. In still other examples, the circularizable probe and the library of splint oligonucleotides are provided in separate compositions which are contacted with the biological sample simultaneously. In some embodiments, the system or kit comprises reagents for generating a rolling circle amplification product (RCP) of the circularized probe in the biological sample, wherein the RCP comprises multiple copies of the gap sequence. In some embodiments, the system or kit comprises reagents for detecting a sequence comprising the variant sequence in the gap sequence of the RCP at a location in the biological sample, thereby detecting the target nucleic acid comprising the variant sequence at the location in the biological sample. The method may but does not need to comprise detecting a barcode sequence in the RCP.

In some embodiments, a system or kit disclosed herein comprises a first probe or probe set comprising a first probe region and a second probe region that hybridize to a first target sequence and a second target sequence, respectively, in a target RNA in the biological sample, wherein the first and second target sequences are separated by a gap sequence in the target RNA, and wherein the gap sequence comprises a variant sequence among a plurality of different variant sequences. In some embodiments, a system or kit disclosed herein comprises reagents for circularizing the first probe or probe set to generate a circularized gap-filled probe comprising a gap-filled region complementary to the gap sequence. In some embodiments, a system or kit disclosed herein comprises reagents for generating a rolling circle amplification product (RCP) of the circularized gap-filled probe in the biological sample, wherein the RCP comprises multiple copies of the gap sequence. For example, the system or kit comprises a polymerase and a plurality of dNTPs.

In some aspects, a system or kit disclosed herein comprises a second probe or probe set that is ligated using the gap sequence in the RCP as a template. In some embodiments, a system or kit disclosed herein comprises reagents for detecting a sequence comprising the variant sequence in the gap sequence of the RCP at a location in the biological sample, thereby detecting the target RNA comprising the variant sequence at the location in the biological sample. In some embodiments, a system or kit disclosed herein comprises reagents for base-by-base sequencing of the sequence comprising the variant sequence, and the base-by-base sequencing may comprise determining the identity of one, two, three, or more bases per cycle in sequential sequencing cycles. For example, the system or kit comprises reagents for performing comprising reagents for performing sequencing-by-synthesis (SBS), sequencing-by-ligation (SBL), sequencing-by-binding (SBB), or sequencing-by-avidity (SBA).

In some aspects, the system or kit comprises a spatial array for analyzing nucleic acid variant sequences of interest in a tissue sample (e.g., as described in Section VI). In some examples, the spatial array comprises an array of features on a substrate, each of which is associated with a unique spatial location on the array. In some instances, the array comprises a plurality of capture agents configured to capture one or more nucleic acid molecules. In some instances, each of the capture agents of a same feature comprises a spatial barcode corresponding to a unique spatial location of the feature on the array. In some embodiments, the capture agents are configured for binding to the ligation product (e.g., generated using the second probe or probe set) or a portion thereof. In some embodiments, the system or kit comprises one or more reagents for permeabilizing and/or lysing a cell or tissue sample, such that the ligation product of a second probe or probe set can be released and/or migrated onto an array surface for capture and spatial barcoding. In some aspects, the ligation product of a second probe or probe set, a barcoded probe targeting the ligation product, and/or a product of the barcoded probe is released and/or migrated onto the spatial array for capture and spatial barcoding. In some aspects, the ligation product of a second probe or probe set and/or a barcoded probe targeting the ligation product comprises features (e.g., capture regions) that are configured to be captured on a spatial array, such that a spatial barcode can be assigned to the ligation product, the barcoded probe, and/or a product thereof for next generation sequencing (NGS) read outs. In some embodiments, a spatially barcoded polynucleotide comprising a barcode sequence (e.g., for identifying a variant sequence of interest) and a spatial barcode can be generated on the array, pooled, and sequenced. In some embodiments, a spatially barcoded polynucleotide, comprising a spatial barcode and a variant sequence of interest or complement thereof can be generated on the array, pooled, and sequenced.

In some embodiments, the systems or kits can contain reagents and/or consumables required for performing one or more steps of the provided methods. In some embodiments, the systems or kits contain reagents for fixing, embedding, and/or permeabilizing the biological sample. In some embodiments, the systems or kits contain reagents, such as enzymes and buffers for ligation and/or amplification, such as ligases and/or polymerases. In some aspects, the system or kit can also comprise any of the reagents described herein, e.g., wash buffer and ligation buffer. In some embodiments, the systems or kits contain reagents for detection and/or sequencing, such as detectably labeled oligonucleotides or detectable labels. In some embodiments, the systems or kits optionally contain other components, for example nucleic acid primers, enzymes and reagents, buffers, nucleotides, modified nucleotides, reagents for additional assays.

### IX. Applications

In some aspects, the provided embodiments can be applied in an in situ method of analyzing nucleic acid sequences in intact tissues or samples in which the spatial information has been preserved. In some alternative aspects, the provided embodiments can be applied in a spatial array method of analyzing nucleic acid sequences in intact tissues or samples in which the spatial information has been preserved. In some aspects, the embodiments can be applied in an imaging or detection method for multiplexed nucleic acid analysis. In some aspects, the provided embodiments can be used to identify or detect mutations in a target nucleic acid. In some aspects, the target nucleic acid is an RNA. In some embodiments, the target nucleic acid is an mRNA. In some aspects, the provided embodiments can be used to crosslink the RCA products via modified nucleotides, e.g., to a matrix, to increase the stability of the circularizable probe or probe set, or the RCA products in situ.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples. In some aspects, the embodiments can be applied to visualize the distribution of genetically encoded markers in whole tissue at subcellular resolution.

### X. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some instances, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The examples are included for illustrative purposes only and are not intended to limit the scope of the present disclosure.

### Example 1: Detecting Single Nucleotide Variations In Situ Using a Gap-Filled First Probe to Generate RCPs and RCP-Templated Ligation of Barcoded Second Probes

This example describes a workflow for KRAS mutation detection in situ including hybridizing a first probe (e.g., a padlock probe) to a target RNA molecule containing a variant sequence (e.g., in a gap sequence), performing a gap fill reaction and ligation to generate a circularized probe comprising a gap-filled region complementary to the gap sequence. The variant sequence in the RCPs (indicated by X as shown in FIG. 4) can be discriminated using ligation of a second probe (e.g., padlock probe) having a ligation site at an interrogatory region for interrogating the variant sequence in the RCP. Barcode sequences associated with the variant sequence in the ligated second probes are detected in situ.

A tissue sample for KRAS mutation analysis is sectioned and the tissue sections are mounted on a slide, fixed (e.g., by incubating in paraformaldehyde (PFA)), washed, and permeabilized (e.g., using Triton-X). After permeabilization, the tissue sections are washed, dehydrated, and rehydrated. The sample comprises target RNA molecules having a wildtype KRAS allele and/or target RNA molecules having a mutant KRAS allele.

A first probe that hybridizes to regions flanking the wildtype and mutant KRAS alleles is designed, and splint oligonucleotides designed to target the wildtype and the mutant KRAS allele can be used to fill the gap the first probe hybridized to a wildtype or mutant KRAS transcript, respectively. The gap-fill padlock probe can but does not need to comprise a barcode region, as shown in FIG. 4. The splint oligonucleotides contain ligatable ends and are ligated to the gap-fill first probe to circularize it for RCA.

The first probe and splint oligonucleotides are applied to the tissue sections and allowed to hybridize at 50° C. overnight, after which the tissue sections are washed and incubated with a ligase (e.g., a SplintR® ligase or T4 RNA ligase 2) in a ligation buffer to form circularized probes. For RCA, the tissue sections are washed and then incubated in an RCA reaction mixture (containing Phi29 reaction buffer, dNTPs, Phi29 polymerase) to generate RCPs containing wildtype or mutant KRAS.

As shown in FIG. 4, second probes are designed to hybridize to the RCPs and comprise interrogatory nucleotides at their 3′ or 5′ ends for discriminating wildtype versus mutant KRAS via probe ligation. The second probes also comprise barcodes associated with the variant sequence. The tissue sections containing RCPs are incubated with a ligase (e.g., a Tth DNA ligase or a Taq DNA ligase) in a ligation buffer to circularize the barcoded second probes. In some instances, linear second probe pairs are used to hybridize to the RCPs and ligated to generate ligated and barcoded second probe pairs.

The barcoded second probes each contains a barcode region comprising one or more barcode sequences that can be detected using detectably labeled probes that bind directly or indirectly to the barcode sequences. As shown in FIG. 4, the detectably labeled probes hybridize to the barcode regions of the ligated (e.g., circularized) barcoded second probes in situ in a hybridization buffer. The tissue sections are washed, stained with DAPI, and mounted in a mounting medium for imaging using fluorescent microscopy to detect the barcode regions corresponding to the wildtype or mutant KRAS.

Detection of KRAS single nucleotide variations using gap-fill first probes (containing common arms) plus splint oligonucleotides that hybridize to RNA transcripts to generate circularized probes for RCA and using barcoded second probes that hybridize to the RCPs (e.g., DNA-templated ligation of the second probe performed by a ligase with high level of specificity) may provide better discrimination between nucleotide variations (e.g., wildtype versus mutant) compared to detection methods using padlock probes that hybridize to RNA transcripts and relies on discrimination of single nucleotide variations using RNA-templated probe ligation.

### Example 2: Detecting Single Nucleotide Variations In Situ Using Barcoded First and Second Probes

This example describes a workflow for KRAS mutation detection in situ including hybridizing a barcoded first probe (e.g., padlock probe) to a target RNA molecule comprising a variant sequence (e.g., in a gap sequence), performing a gap fill reaction and ligation to generate a circularized gap-filled probe comprising a gap-filled region complementary to the gap sequence. The variant sequence in the RCPs are discriminated using ligation of a second probe as described in Example 1. In addition, barcode sequences in the RCPs are detected in addition to barcode sequences in the ligated second probes to detect the single nucleotide variations in situ.

A tissue sample is prepared as described in Example 1 and a first probe hybridizes to regions flanking the wildtype and mutant KRAS alleles and splint oligonucleotides as described in Example 1 are designed. The first probe comprises a barcode region, as shown in FIG. 5, and the barcode region can comprise four overlapping barcode sequences and can be used to identify KRAS from other gene transcripts. The splint oligonucleotides contain ligatable ends and are ligated to the gap-fill padlock probe to circularize it for RCA. Hybridization, gap-fill and ligation of the barcoded first probe is performed as described in Example 1 to generate RCPs containing wildtype or mutant KRAS.

As shown in FIG. 5, barcoded second probes hybridize to the RCPs and the barcoded probes comprise interrogatory nucleotides at their 3′ or 5′ ends for discriminating wildtype versus mutant KRAS via probe ligation. The tissue sections containing RCPs are incubated with a ligase (e.g., a Tth DNA ligase or a Taq DNA ligase) in a ligation buffer to circularize the barcoded second probes. In some instances, linear second probe pairs are used to hybridize to the RCPs and ligated to generate barcoded second probe pairs.

The barcoded second probes each contains a barcode region comprising one or more barcode sequences corresponding to a base (e.g., barcode regions corresponding to A, U, C, or G in RNA transcripts). The barcode regions in the RCPs and the barcode regions in the ligated second probes are detected using detectably labeled probes that bind directly or indirectly to the barcode sequences. As shown in FIG. 5, the detectably labeled probes hybridize to the barcode regions of the RCP and the ligated second probes in situ in a hybridization buffer. The tissue sections are washed, stained with DAPI, and mounted in a mounting medium for imaging using fluorescent microscopy to detect the barcode regions in the RCPs (to identify the transcripts as KRAS versus other gene transcripts) and to detect the barcode regions in the ligated second probes (to identify wildtype versus mutant KRAS transcripts).

Detection of KRAS single nucleotide using DNA-templated ligation of the second probe performed by a ligase with high level of specificity may provide better discrimination between nucleotide variations (e.g., wildtype versus mutant) compared to detection methods using padlock probes that hybridize to RNA transcripts and relies on discrimination of single nucleotide variations using RNA-templated probe ligation. In some instances, the barcode sequences in RCPs and the second probes allow the use of common barcode sequence(s) to distinguish the identity of the variant sequence among different genes (e.g., identify wildtype versus mutant SNPs across multiple genes) since the identity of the gene is provided by detecting the barcode regions in the RCPs.

### Example 3: Detecting Single Nucleotide Variations Using a Gap-Filled Probe to Generate RCPs, Probe Ligation Templated on the RCPs, and Capturing the Ligated Probes on a Spatial Array

This example describes a workflow for KRAS mutation detection including hybridizing a first probe (e.g., padlock probe) to a target RNA molecule containing a variant sequence (e.g., gap sequence), performing a gap fill reaction and ligation to generate a circularized gap-filled probe comprising a gap-filled region complementary to the gap sequence. The variant sequence in the RCPs are discriminated using ligation of a second probe set and capturing the ligated probe product on a spatial array for spatial barcoding and subsequent sequencing.

A tissue sample for KRAS mutation analysis is prepared and contacted with the first probe as described in Example 1, the first probe is gap filled, ligated, and amplified to generate an RCP. As shown in FIG. 7, a second probe set is hybridized to the RCP. Each second probe set is a pair of probes that hybridize to adjacent sequences flanking the single nucleotide variation position in the RCP, and one or the probes of the probe pair comprises an interrogatory nucleotide at the 3′ or 5′ end for discriminating wildtype versus mutant KRAS (e.g., indicated as X or Y in FIG. 7) via probe ligation. The tissue section containing RCPs are incubated with a ligase (e.g., a Tth DNA ligase or a Taq DNA ligase) in a ligation buffer to ligate the probe pairs.

In some instances, a first probe of the probe pair comprises a functional sequence (e.g., Read 2S) at its 5′ end and a sequence at the 3′ end comprising the interrogatory nucleotide for the single nucleotide variation in the RCP. The second probe of the probe pair comprises i) a capture sequence that is complementary to a capture domain of a capture probe on a spatial array, and ii) a sequence that is complementary to the RCP at its 5′ end. Following probe hybridization, the two probes are ligated together to generate a ligation product (e.g., ligated second probe set) that serves as a proxy of the wildtype or mutant KRAS. One or more washes are performed during probe pair hybridization, and following probe pair ligation, the tissue sections are washed one or more times.

The ligation products are released from the RCPs and migrated to an arrayed slide for capture by the capture domains of the capture probes on the spatial array. A capture probe of the hydrogel barcoded capture array comprises a functional sequence (e.g., Read 1 primer sequence), a spatial barcode sequence, a unique molecular identifier or UMI sequence, and a capture domain, for instance a poly(dT)VN sequence at the 3′ end. In this example, the capture domain in the capture probe captures the ligation product by hybridization of the poly(dT)VN sequence to the poly(A) domain of the ligation product.

Following capture of ligated probes, a spatially labeled polynucleotide is generated and sequenced to determine all or a portion of the ligated probe sequence or complement thereof (which is a proxy of the target RNA sequence, such as wildtype or mutant KRAS transcripts) and all or a portion of the spatial barcode sequence or complement thereof (which provides the spatial information of the target RNA sequences in the tissue sections) in the same spatially labeled polynucleotide molecule. After hybridization, the capture probe and ligation product are extended, the extended ligation product is released from the spatial array and library preparation steps are performed in anticipation of downstream sequence analysis, which associates the spatial barcodes and target RNA sequences (e.g., wildtype or mutant KRAS) in the sequencing reads of the spatially labeled polynucleotides with the locations in the tissue sections, thereby identifying the variant sequences of one or more target RNAs at locations in the tissue sections.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1-135. (canceled)

136. A method, comprising:
a) contacting the biological sample with a first probe or probe set,
wherein the first probe or probe set comprises a first probe region and a second probe region that bind to a first target sequence and a second target sequence, respectively, in a target RNA in the biological sample,
wherein the first and second target sequences flank a gap sequence in the target RNA, and
wherein the gap sequence comprises a variant sequence;
b) performing a gap-fill reaction on the first probe or probe set bound to the target RNA to generate a gap-filled probe or probe set and circularizing the gap-filled probe or probe set;
c) using a polymerase to amplify the circularized gap-filled probe or probe set to generate a rolling circle amplification product (RCP) in the biological sample;
d) contacting the biological sample with a second probe or probe set that binds to the RCP, wherein the second probe or probe set comprises an interrogatory region for interrogating the variant sequence in the RCP;
e) ligating the second probe or probe set bound to the RCP to generate a ligation product comprising the interrogatory region which comprises a sequence complementary to the variant sequence in the RCP; and
f) detecting the ligation product to determine a location of the target RNA comprising the variant sequence in the biological sample.

137. The method of claim 136, wherein performing the gap-fill reaction comprises contacting the biological sample with a library of splint oligonucleotides, wherein each splint oligonucleotide comprises:
i) ligatable ends; and
ii) a hybridization region complementary to one of a plurality of different sequences,
wherein a splint oligonucleotide of the library of splint oligonucleotides that is complementary to the gap sequence is ligated to the first probe or probe set.

138. The method of claim 137, wherein the variant sequence is at least 1 or more phosphodiester bonds from the 3′ or 5′ end of the gap sequence, and/or wherein the sequence complementary to the variant sequence is at least 1 or more phosphodiester bonds from the 5′ or 3′ end of the splint oligonucleotide.

139. The method of claim 137, wherein the splint oligonucleotide is ligated to the first probe or probe set using the target RNA as a template and a ligase having an RNA-templated DNA or RNA ligase activity.

140. The method of claim 137, comprising washing the biological sample after contacting with the library of splint oligonucleotides.

141. The method of claim 136, wherein performing the gap-fill reaction comprises using a gap-fill polymerase to extend an end of the first probe or probe set using the target RNA as a template to generate an extended probe, wherein the extended probe is ligated to another end of the first probe or probe set.

142. The method of claim 136, wherein the variant sequence comprises a single nucleotide of interest, the interrogatory region in the second probe or probe set comprises a nucleic acid residue complementary to the single nucleotide of interest, and the nucleic acid residue is no more than 5 phosphodiester bonds from a 3′ or 5′ end of the second probe or probe set.

143. The method of claim 142, wherein the interrogatory region comprises a 3′ terminal nucleic acid residue that is complementary to the single nucleotide of interest.

144. The method of claim 142, wherein the interrogatory region comprises a 5′ terminal nucleic acid residue that is complementary to the single nucleotide of interest.

145. The method of claim 136, wherein the second probe or probe set is ligated using a ligase having a DNA-templated DNA ligase activity.

146. The method of claim 136, wherein the first probe or probe set comprises a barcode region comprising one or more barcode sequences associated with the target RNA or a sequence thereof, wherein the barcode region is not complementary to the target RNA or sequence thereof.

147. The method of claim 136, wherein the second probe or probe set comprises a barcode region comprising one or more barcode sequences associated with the target RNA or a sequence thereof, wherein the barcode region is not complementary to the target RNA or sequence thereof.

148. The method of claim 136, wherein the second probe or probe set comprises the interrogatory region and a constant region complementary to the target RNA.

149. The method of claim 148, wherein the constant region is common among a plurality of second probes or probe sets each comprising a different interrogatory region for a different variant sequence of the target RNA.

150. A method, comprising:
a) contacting the biological sample with a first probe or probe set,
wherein the first probe or probe set comprises a first probe region and a second probe region that bind to a first target sequence and a second target sequence, respectively, in a target RNA in the biological sample,
wherein the first and second target sequences flank a gap sequence in the target RNA, and wherein the gap sequence comprises a variant sequence;
b) performing a gap-fill reaction on the first probe or probe set bound to the target RNA to generate a gap-filled probe or probe set and circularizing the gap-filled probe or probe set;
c) using a polymerase to amplify the circularized gap-filled probe or probe set to generate a rolling circle amplification product (RCP) in the biological sample;
d) contacting the biological sample with a second probe or probe set that binds to the RCP, wherein the second probe or probe set comprises i) an interrogatory region for interrogating the variant sequence in the RCP, and ii) a barcode region;
e) ligating the second probe or probe set bound to the RCP to generate a ligation product when the interrogatory region comprises a sequence complementary to the variant sequence in the RCP; and
f) contacting the biological sample with detectably labeled probes in sequential cycles, wherein in each cycle, a detectably labeled probe is directly or indirectly bound to a barcode sequence in the barcode region and a signal or absence thereof associated with the detectably labeled probe is recorded at a location in the biological sample, thereby generating a signal code sequence corresponding to the barcode region, wherein the signal code sequence comprises the signal or absence thereof recorded at the location in each of the sequential cycles;
wherein the signal code sequence identifies the variant sequence of the target RNA at the location in the biological sample.

151. The method of claim 150, wherein the barcode region in the second probe or probe set is associated with the variant sequence in the target RNA.

152. The method of claim 150, wherein in e), the biological sample is contacted with a plurality of second probes or probe sets each comprising a different interrogatory region for a different variant sequence of the target RNA and a different barcode region corresponding to the different variant sequence.

153. The method of claim 150, wherein the first probe or probe set comprises a barcode region comprising one or more barcode sequences associated with the target RNA or a sequence thereof but not associated with the variant sequence.

154. The method of claim 150, wherein in each cycle, the detectably labeled probe is hybridized to an intermediate probe which in turn hybridizes to the barcode region.

155. The method of claim 136, wherein the biological sample is a cell or tissue sample comprising cells or cellular components.
